(19) United States
(12) Patent Application Publication
(10) Pub. No.: US 2006/0194230 A1
(43) Pub. Date: Aug. 31, 2006

(54) Genetic markers associated with benign prostatic hyperplasia

(75) Inventors: Jonathan Levitt, Houston, TX (US); Kevin M. Slawin, Houston, TX (US); Eduardo Canto, Houston, TX (US); David M. Spencer, Houston, TX (US); Michael Ittmann, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/339,733

(22) Filed: Jan. 25, 2006

Related U.S. Application Data
(60) Provisional application No. 60/646,841, filed on Jan. 25, 2005.
(60) Provisional application No. 60/646,659, filed on Jan. 25, 2005.

(51) Int. Cl. C12Q 1/68; G01N 33/574

(52) U.S. Cl. 435/6; 435/7.23

(57) ABSTRACT

The present invention regards expression profiles of one or more nucleic acids indicative of the presence of, susceptibility to, and/or predicting response to therapy of benign prostatic hyperplasia (BPH) in an individual. The present invention identifies pathways not previously associated with BPH, therefore presenting novel diagnostic and therapeutic targets for the condition.

The present invention claims priority to U.S. Provisional Application Ser. No. 60/646,659 filed Jan. 25, 2005, and U.S. Provisional Application Ser. No. 60/646,841, filed Jan. 25, 2005, both of which are incorporated by reference herein in their entirety.

### STATEMENT REGARDING FEDERALLY SPONSORED RESEARCH OR DEVELOPMENT

The present invention utilized funds from the National Institutes of Health (NIH) National Institute of Diabetes, Digestive and Kidney Diseases (NIDDK) Grant No. 5U01 DK63594. The United States Government may have certain rights in the invention.

### FIELD OF THE INVENTION

The present invention concerns at least the fields of molecular biology, cell biology, and medicine.

### BACKGROUND OF THE INVENTION

Benign prostatic hyperplasia (BPH) is a common affliction of the aging male, causing significant morbidity and health care expenditures in the United States and around the world. BPH refers to a constellation of problems that include lower urinary tract symptomatology (LUTS) and lower urinary tract obstruction associated with BPH-related prostate enlargement. As men pass the age of ˜40 years, the transition zone of the prostate begins to exhibit pathologic changes (e.g. mixed stromal and epithelial nodule formation) that is directly related to transition zone and total prostate gland enlargement. The prostate gland, which measures approximately 20 cc in the young, healthy male, can grow to over 200 cc, a greater than 10-fold increase, in men severely affected by pathology BPH. This prostate growth and enlargement is highly associated with clinical progression of BPH, including worsening LUTS, urinary retention, and the need for BPH-related surgery.

Through translational research efforts over the past 3 decades, important pathways have been identified that impact on disease severity and progression, leading to effective medical therapies for BPH. Due to the identification of the 5-alpha reductase enzyme in the prostate and the recognition of the importance of its function in the establishment and progression of BPH, 5 alpha reductase inhibitors like finasteride and dutasteride have been developed to treat men with BPH. Similarly, the identification of alpha-1 adrenergic receptors in the prostate and the delineation of their role in BPH has led to the development of alpha blockers like terazosin, doxazosin, alfuzosin and tamsulosin for the treatment of BPH. Both these classes of drugs are now widely utilized for the treatment of BPH.

Furthermore, the identification of prostate-specific and BPH-associated markers like PSA and BPSA have also improved the ability to diagnose and manage patients with BPH.

U.S. Pat. No. 5,912,135 describes BPH diagnosis without requiring a biopsy. The total prostate specific antigen (PSA) level in the blood or serum of the patient is measured. If the patient has a total PSA level of between about 2.5 ng/ml and 10.0 ng/ml, then the free PSA level in the blood or serum of the patient is measured. The proportion of free PSA to total PSA is calculated. If this proportion is equal to or greater than about 25%, then the patient is diagnosed as having benign prostatic diseases (BPD). Optionally, if the patient has a total PSA level of between 10.1 ng/ml and 20.0 ng/ml, then the free PSA level in the blood or serum of the patient can also be measured. The proportion of free PSA to total PSA is calculated. If this proportion is equal to or greater than about 25%, then the patient is diagnosed as having BPD.

Therefore, the identification of new BPH-disease-related pathways and markers may provide the opportunity to develop new, effective therapies targeted at these pathways and/or to monitor therapy for BPH as well as to develop new diagnostic markers.

### BRIEF SUMMARY OF THE INVENTION

The present invention concerns the diagnosis and/or treatment of benign prostatic hyperplasia (BPH). In particular, one or more polynucleotides are associated with the risk of developing BPH in an individual, which may be further defined as being susceptible to developing BPH or having an elevated chance of developing BPH. In other embodiments, one or more polynucleotides are associated with the identification of BPH present in an individual. In certain aspects, polynucleotides of the present invention are employed for the diagnosis of BPH and/or for the treatment of BPH. In further specific embodiments, the polynucleotides of the present invention are employed for predicting response to a BPH therapy.

In particular aspects of the invention, the level of one or more polynucleotides, and/or the encoded product thereof, is indicative for an individual of a risk of developing BPH or the identification of presently occurring BPH in an individual.

In particular, the nature of the polynucleotides and their encoded products may reflect the etiology of BPH, and in specific aspects of the invention they relate to certain molecular biological pathways in a cell of the individual, such as a prostate cell, for example. In specific embodiments of the invention, the pathway may be an inflammatory pathway, a Wnt pathway, a cell signaling or cycling pathway, extracellular matrix remodeling pathway, or a combination thereof. In alternative embodiments, the polynucleotide(s) and their encoded products are not associated with a particular pathway. However, in some embodiments of the invention, a combination of one or more polynucleotides from one or more of the pathways are diagnostic or therapeutic for BPH.

In further specific embodiments of the invention, the RNAs are expressed from one or more polynucleotides listed herein in Table 3, Table 4, Table 5, Table 9, or a combination thereof.

In an embodiment of the present invention, there is a method of identifying a risk of developing benign prostatic hyperplasia (BPH) and/or detecting the presence of BPH in an individual, comprising the step of identifying a change in a level of one or more polynucleotides or an encoded product thereof, wherein the encoded product is a member of an inflammatory pathway, a Wnt signaling pathway, a cell signaling pathway, a cell cycle pathway, or a combination thereof. In a specific embodiment, the polynucleotide or encoded product is identified in Table 3, Table 4, Table 5, Table 9, or a combination thereof. The identifying step may be further defined as comprising obtaining a sample from the individual and detecting a change in level of one or more nucleic acid sequences or the encoded product thereof in Table 3, Table 4, Table 5, Table 9, or a combination thereof.

In specific embodiments of the invention, samples comprise biopsy, needle aspirate, prostate fluid, serum, blood, and/or urine, for example. In particular embodiments, the individual has a prostate size larger than about 30 grams. Detecting steps may comprise microarray analysis, polymerase chain reaction, immunoblot analysis, immunoassay, proteomic assay, or a combination thereof, for example.

In some embodiments of the invention, methods provided herein further comprise evaluating an additional risk factor of the individual, such as age, race, total PSA level, free PSA level, % Free PSA, BPSA level, -2proPSA level, maximum urine flow rate, AUA SI, BPH impact index, PVR, ultrasound total prostate volume, ultrasound TZ volume, or a combination thereof. In particular embodiments, the individual is identified as being at risk for developing BPH or is identified as having BPH, the individual is administered a therapy. As an example, the therapy may comprise surgery, or the therapy may be a minimally invasive therapy, such as microwave treatment, radiofrequency treatment, delivery of therapeutic composition, or a combination thereof.

In an additional embodiment, there is a method of treating an individual for BPH, comprising the step of providing to the individual an agent that targets an inflammatory pathway, a Wnt pathway, a cell signaling pathway, a cell cycle pathway, or a combination thereof. The agent may comprise an antibody, a small molecule, antisense RNA, a protein, or a mixture thereof. In specific embodiments, the agent targets a polynucleotide or the encoded product thereof identified in Table 3, Table 4, Table 5, Table 7, Table 9, or a combination thereof.

In one embodiment of the present invention, there is a method of predicting or evaluating the response of an individual to a BPH therapy and/or identifying a risk of developing BPH, and/or detecting the presence of BPH, comprising the step of providing the level of one or more expressed RNAs from an individual prior to the BPH therapy and/or during the BPH therapy. The providing the level step may be further defined as providing the level of at least some of the one or more expressed RNAs or gene products encoded therefrom from the individual. There may also be comparison to the level of at least one RNA from a standard or known control. In a specific embodiment, the level of one or more expressed RNAs from the individual is upregulated compared to a known standard or control or an individual that does not have BPH. In alternative specific embodiments, the level is downregulated. In other specific embodiments, the level of expression is alternatively evaluated with the level of the encoded gene product, such as the encoded protein level.

In particular embodiments, the level of one or more polynucleotides or encoded products thereof is from an individual prior to BPH therapy and/or during BPH therapy. In particular, the level of the polynucleotides or encoded products are indicative of a response to the therapy. The response to the therapy may provide information concerning resistance or ineffectiveness of the individual to the therapy or the response may provide information concerning sensitivity or effectiveness to the therapy. In a specific embodiment, the levels of one or more RNAs from an individual sensitive to the therapy is provided at least in part from a known standard.

In a specific embodiment, providing the level of RNAs from the individual comprises the following steps: obtaining one or more cells from the individual; isolating RNA from the one or more cells; and determining the level of one or more of the RNAs or encoded products thereof. In further specific embodiments, the RNA levels are determined by microarray analysis. The cells may be obtained from prostate tissue, serum, blood, urine, and so forth.

In specific embodiments of the invention, the RNAs are expressed from one or more polynucleotides and their encoded products concern an inflammatory pathway, a Wnt pathway, an extracellular matrix remodeling pathway, a cell cycle pathway, a cell signaling pathway, or a combination thereof. In specific embodiments, the one or more polynucleotides and their encoded products are listed herein in Table 7. In additional specific embodiments, when the method predicts the therapy as being non-effective for BPH, the individual is subjected to an alternative therapy, such as one comprising an alternative drug therapy, microwave radiation, radiofrequency treatment, surgery, gene therapy, or a combination thereof.

In another specific embodiment, the difference between the level of at least one expressed polynucleotide in the individual and the control is greater than about one-fold.

In an embodiment of the present invention, there is a method of identifying a risk of developing benign prostatic hyperplasia (BPH) and/or detecting the presence of BPH in an individual or the method may be for predicting or evaluating the response of an individual to a BPH therapy, comprising the step of identifying a change in a level of one or more polynucleotides or an encoded product thereof, wherein the encoded product is a member of an inflammatory pathway, a Wnt pathway, a cell signaling pathway, a cell cycle pathway, an extracellular matrix remodeling pathway, or a combination thereof. The method may be further defined as comprising the steps of providing the level of one or more expressed polynucleotides from an individual who is to receive a BPH therapy or who is receiving a BPH therapy; and comparing the level of one or more expressed polynucleotides to a control, wherein a difference between the level of at least one expressed polynucleotide predicts the response to BPH therapy in the individual. In specific embodiments, the BPH therapy is further defined as a 5-alpha reductase inhibitor, an alpha-I adrenergic receptor antagonist, or a mixture thereof. In specific embodiments, the BPH therapy is finasteride, tamsulosin, or a mixture thereof. In specific embodiments, the one or more polynucleotides or encoded products thereof are identified in Table 7.

In specific embodiments, one or more identifying steps of any of the methods of the invention are further defined as comprising obtaining a sample from the individual; and detecting a change in expression of one or more nucleic acid sequences in Table 7. Samples may comprise biopsy, prostate fluid, serum, blood, urine, seminal fluid, or a combination thereof. The identifying step may comprise identifying an expressed RNA level of the one or more nucleic acid sequences, an expressed protein level encoded by the one or more nucleic acid sequences, or both, and it may be further defined as comprising microarray analysis, polymerase chain reaction, immunoblot, or a combination thereof.

In particular aspects of the invention, the difference between the levels is defined as being higher in the individual than the control, as being lower in the individual than the control, or a combination of expressed polynucleotides being higher or lower in the individual as compared to the control. In exemplary embodiments, the difference between the level of at least one expressed polynucleotide in the individual and the control is greater than about one-fold. Exemplary polynucleotides are provided in Table 7.

In specific embodiments, providing the level of the expressed polynucleotides is further defined as providing the level of expressed RNAs and/or is further defined as providing the level of expressed proteins. RNA levels may be determined by microarray analysis, quantitative polymerase chain reaction, or both, for example. Protein levels may be determined by immunoblot, for example.

In particular embodiments wherein the method predicts the individual as being refractory to the BPH therapy, the individual may be subjected to an alternative BPH therapy, such as surgery and/or a minimally invasive therapy, including microwave treatment, radiofrequency treatment, therapeutic composition treatment, or a combination thereof.

In another embodiment of the present invention, there is a method of determining performance for one or more drug therapies for BPH in an individual, comprising the step of identifying the expression level of one or more polynucleotides in Table 7 in the individual.

The method may be further defined as comparing the level of one or more nucleic acid sequences in Table 7 in one or more prostate cells of the individual with the level of one or more nucleic acid sequences in Table 7 from one or more cells that are known to be sensitive to the BPH drug therapy. In specific embodiments, when the level in one or more of the nucleic acid sequences in Table 7 of the individual is higher than the level in one or more cells that are sensitive to the BPH therapy, the individual is sensitive to the BPH therapy. In further specific embodiments, when the level in one or more of the nucleic acid sequences in Table 7 of the individual is lower than the level in one or more cells that are sensitive to the BPH therapy, the individual is sensitive to the BPH therapy. In additional specific embodiments, when the level in one or more of the nucleic acid sequences in Table 7 of the individual is higher than the level in one or more cells that are sensitive to the BPH therapy, the individual is resistant to the BPH therapy. In further specific embodiments, when the level in one or more of the nucleic acid sequences in Table 7 of the individual is higher than the level in one or more cells that are sensitive to the BPH therapy, the individual is resistant to the BPH therapy.

In another embodiment, there is as a composition of matter, isolated expressed polynucleotides the levels of which are indicative of a risk for developing benign prostatic hyperplasia (BPH), and/or the presence of BPH. In specific embodiments, one or more of the expressed polynucleotides are identified in Table 3, Table 4, Table 5, or Table 9. The expressed polynucleotides may be comprised on a substrate, such as a microarray chip.

Compositions of the present invention may further comprise BPH risk factor-evaluating information, one or more BPH risk factor-evaluating reagents, or a combination thereof the BPH risk factor-evaluating information may comprise, for example, statistical information for prostate related to age, race, total PSA level, free PSA level, % Free PSA, BPSA level, -2proPSA level, maximum urine flow rate, AUA SI, BPH impact index, PVR, ultrasound total prostate volume, ultrasound TZ volume, or a combination thereof. In specific embodiments, the BPH risk factor-evaluating reagent comprises a PSA level measuring reagent, a maximum urine flow rate measuring reagent, or both.

Therapeutic compositions of the invention may target a member of an inflammatory pathway, a Wnt signaling pathway, a cell signaling pathway, a cell cycle pathway, or a combination thereof, in specific embodiments. In specific embodiments, the therapeutic composition comprises antisense RNA, an antibody, a small molecule, a protein, or a mixture or combination thereof.

In an additional embodiment of the present invention, there is a pharmaceutical composition, wherein the composition is capable of treating benign prostatic hyperplasia and comprises an agent that targets a member of an inflammatory pathway, a Wnt signaling pathway, a cell signaling pathway, a cell cycle pathway, or a combination thereof. The composition may be further defined as the agent targeting the expression of a polynucleotide or an encoded product thereof, wherein the polynucleotide is identified in Table 3, Table 4, Table 5, or Table 9. In specific embodiments, the composition is an antibody, an antisense RNA, a small molecule, or a mixture or combination thereof, for example. The composition may be comprised in a pharmaceutically acceptable excipient.

Compositions of the present invention may be provided in a kit, such as a diagnostic and/or therapeutic kit, housed in a suitable container. In another embodiment, there is a kit housed in a suitable container, comprising a composition that identifies expression level of one or more polynucleotides of Table 3, 4, 5, 7, or 9. The kit may include compositions that are employed for diagnosis, such as the polynucleotides of the present invention provided on a substrate, including a microchip, blot or gel; nucleic acids, such as those that target a polynucleotide, for example, such as primers for polymerase chain reaction; or a combination thereof. The kit may also have BPH risk factor information, such as in a pamphlet, and/or risk factor reagent, such as reagents and/or apparatus to detect PSA level, for example. In specific embodiments, the composition further comprises BPH risk factor-evaluating information, BPH risk factor-evaluating reagent, or a combination thereof. In specific embodiments, the BPH risk factor-evaluating information comprises statistical information for prostate related to age, race, total PSA level, free PSA level, % Free PSA, BPSA level, -2proPSA level, maximum urine flow rate, AUA SI, BPH impact index, PVR, ultrasound total prostate volume, ultrasound TZ volume, or a combination thereof. In specific embodiments, the BPH risk factor-evaluating reagent comprises a PSA level measuring reagent, a maximum urine flow rate measuring reagent, or both. The kit may also comprise an agent that targets the polynucleotide, an encoded product of the polynucleotide, or both. In specific embodiments, the agent is an antibody, a small molecule, antisense RNA, siRNA, protein, peptide, or a mixture or combination thereof. The agent may be provided in a pharmaceutically acceptable excipient, in specific embodiments.

In another embodiment of the invention, there is as a composition of matter expressed RNAs the levels of which are indicative of a susceptibility to BPH and/or the presence of BPH and/or a response to BPH therapy, wherein one or more of the expressed RNAs or gene products thereof, further wherein they are members of an inflammatory pathway, a cell signaling pathway, a cell cycle regulatory pathway, or an extracellular matrix remodeling pathway. In specific embodiments, they are listed herein in Table 7. In specific embodiments, the expressed RNAs are comprised on a substrate, such as a microarray chip, a gel, or a blot, for example. In alternative embodiments, the composition of matter comprises gene products, such as polypeptides or proteins, the levels of which are indicative of BPH susceptibility or presenct of BPH.

In an additional embodiment, there is a kit housed in a suitable container, comprising an agent that targets a polynucleotide identified in Table 3, 4, 5, 7, or 9 or the encoded product thereof. The agent may be provided in a pharmaceutically acceptable excipient. The agent may be an antibody, a small molecule, antisense RNA, siRNA, protein, peptide, nucleic acid, or a mixture or combination thereof.

In specific embodiments of the present invention, the inventors employed the exemplary Affymetrix U133 Plus 2.0 whole genome microarrays to profile gene expression in the transition zone (TZ) of small (≦30 g) and big (≧70 g) prostates. In addition, they also profiled gene expression changes in TZ benign prostatic hyperplasia (BPH) tissue from patients treated with α1-receptor blockade (tamsulosin), 5α-reductase type II inhibition (finasteride), or both medications combined. Integrating the results from these profiles led to the discovery of 5 exemplary polynucleotides that in specific embodiments are diagnostic and/or prognostic biomarkers for BPH. These exemplary markers include the three interferon inducible pro-inflammatory chemokines, MIG (CXCL9), IP 10 (CXCL10), and I-TAC (CXCL11), the adhesion molecule Contactin-1 (CNTN1), the Wnt pathway signaling inhibitor Dickkopf-3 (DKK3), and any combination thereof. These five markers are up-regulated in untreated BPH tissue, and their expression is correspondingly decreased by treatment with tamsulosin or finasteride, for example. The expression of these polynucleotides was further characterized using real-time quantitative reverse transcriptase polymerase chain reaction, for example, and their protein production was also demonstrated in whole prostate tissue and in exemplary prostate cell lines. Further, the inventors demonstrated that the CXCL chemokines are detectable in serum and urine, for example, of patients with prostate pathology. In further specific embodiments, the mechanisms by which these inflammatory BPH markers are induced and their influence on prostate hyperplasia is determined.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 illustrates an exemplary inflammatory pathway in a human cell.

FIG. 2 illustrates an exemplary Wnt pathway in a human cell.

FIG. 3 illustrates an exemplary cell signaling and cell cycle pathway.

FIG. 4 illustrates an exemplary randomized block design with variable block size for randomly assigning participants to nine treatment groups.

FIG. 5 shows expression of CXCL9 in prostate tissue. BPH transition zone tissue (TZ) and adjacent peripheral zone tissue (PZ) from the same individual are stained with a mouse mAb for CXCL9. Left panel shows background staining in TZ tissue with isotype control immunoglobulin. All images were recorded with the same exposure and illumination settings.

FIG. 6 shows expression of CXCL10 in prostate tissue. BPH transition zone tissue (TZ) and adjacent peripheral zone tissue (PZ) from the same individual are stained with a polyclonal goat serum for CXCL10. Left panel shows background staining in TZ tissue with non-immune goat serum. All images were recorded with the same exposure and illumination settings.

FIG. 7 shows expression of CXCL11 in prostate tissue. BPH transition zone tissue (TZ) and adjacent peripheral zone tissue (PZ) from the same individual are stained with a polyclonal goat serum for CXCL11. Left panel shows background staining in TZ tissue with non-immune goat serum. All images were recorded with the same exposure and illumination settings.

FIG. 8 shows expression of Contactin 1 in prostate tissue. BPH transition zone tissue (TZ) and adjacent peripheral zone tissue (PZ) from the same individual are stained with a polyclonal goat serum for Contactin 1. Left panel shows background staining in TZ tissue with non-immune goat serum. All images were recorded with the same exposure and illumination settings.

FIG. 9 demonstrates expression of DKK3 in prostate tissue. BPH transition zone tissue (TZ) and adjacent peripheral zone tissue (PZ) from the same individual are stained with a polyclonal goat serum for DKK3. Left panel shows background staining in TZ tissue with non-immune goat serum. All images were recorded with the same exposure and illumination settings.

FIG. 10 shows expression of CXCR3 in prostate tissue. BPH transition zone tissue (TZ) and adjacent peripheral zone tissue (PZ) from the same individual are stained with a mouse mAb for CXCR3. Two individual BPH patients are shown in the upper panels with their corresponding normal tissue below. All images were recorded with the same exposure and illumination settings.

FIG. 11 demonstrates expression of potential BPH markers in prostate cell lines. Cells were permeablized and stained for intracellular chemokines CXCL9, 10 and 11 or surface markers contactin 1 and chemokine receptor CXCR3. Bar height represents the mean fluorescent intensity (MFI) of each marker in each cell line minus the MFI for the cells stained with an isotype matched control. ND—not determined for cell lines not stained for a specific marker.

FIG. 12 shows that CXCL chemokines are up-regulated by IFNγ, in prostate cells. 4 prostate cell lines were cultured in media with with or without 100 U/ml IFN\tilde{γ}. Chemokine expression was determined by Q-RT-PCR and induction calculated using the 2^{-ΔΔCt} method compared to untreated cells.

FIG. 13 shows that CXCL chemokines 9, 10 and 11 are detectable in serum and urine from patients with prostate disease. Serum from patients with BPH, prostate cancer or normal controls (n=13 per group) and urine from patients with BPH or prostate cancer (n=20 per group) were measured for the presence of CXCL9, 10 and 11 by ELISA. All samples were measured in duplicate. Error bars represent the standard error of the mean for the group.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The term “expressed RNAs” as used herein refers to RNAs that are transcribed from a polynucleotide. In specific embodiments, the polynucleotide is a gene, such as a gene on a chromosome or mitochondrial DNA. In further embodiments, the expressed RNAs are isolated from one or more cells, such as one or more prostate cells suspected of coming from an enlarged prostate. In specific embodiments, the level of the expressed RNA may be determined by determining the level of a polypeptide translated from the expressed RNA, such as determining the level by immunoblot.

The term “genetic marker” as used herein refers to a polynucleotide having a known identity and associated with a particular condition. In particular embodiments, the genetic marker comprises the expression level of the polynucleotide, which may be determined as an RNA level and/or a protein level. Genetic markers associated with the condition of benign prostatic hyperplasia can be detected by any suitable means and from any suitable sample. In particular embodiments, the marker determines whether an individual is at risk for developing BPH, is susceptible to developing BPH, or has BPH.

The term “predicting” as used herein refers to identifying a chance of developing BPH.

The term “RNA expression profiling” or “RNA expression profile” as used herein refers herein to collecting information from a plurality of expressed genes in the form of RNA transcripts, or the collection thereof, respectively. In specific embodiments, the plurality of RNA transcripts provides information related to benign prostate hyperplasia therapy. In additional specific embodiments, the information gleaned from profiling facilitates determination of a benign prostate hyperplasia therapy, such as whether or not to employ a particular therapy. In particular embodiments, the collection of expressed genes is compared between two samples, and in specific embodiments those samples are from one or more individuals suspected of developing or having BPH. In specific embodiments, the comparison provides information whether or not a particular treatment should be utilized or continued for the individual.

The term “microarray” as used herein refers to a collection of expressed RNAs, in particular comprised on a substrate, such as a microchip.

The term “minimally invasive” as used herein refers to a procedure for treating BPH that does not require anesthesia, an incision, or both. The procedure may be performed in the office of a doctor, as opposed to an operating room of a hospital. The treatment may comprise microwave treatment, radiofrequency treatment, or both.

The term “pathway” as used herein refers to two or more gene products that work to signal sequentially and/or in branched form through to some cellular and/or molecular biological effect, such as gene expression of a particular polynucleotide and/or release of other signaling mediators.

The term “resistance” as used herein refers to when a therapy for an individual may be ineffective. In specific embodiments, the ineffectiveness is detected by identifying the expression level of one or more polynucleotides or encoded products therefrom. In further specific embodiments, the ineffectiveness of the therapy is further evaluated by identifying growth of the prostate, such as resumed or continued enlargement.

The term “suspected of developing benign prostatic hyperplasia” as used herein refers to an individual that may have potential for developing BPH, and in particular that individual is a human male over the age of about 40.

II. The Present Invention

In certain aspects of the invention, the methods and compositions relate to obtaining prostate information for any individual, regardless of whether or not the individual has any abnormality associated with the prostate. In other aspects of the invention, the methods and compositions relate to obtaining prostate information for an individual known to have BPH, suspected of having BPH, or suspected of developing BPH.

Benign prostatic hyperplasia (BPH) refers to the benign enlargement of the prostate that develops in the aging male population. BPH is characterized by the development of mixed stromal and epithelial nodules within the transition zone of the prostate that are associated with prostate growth and the development of lower urinary tract symptoms in aging men. The standard medical therapies for BPH are selective α₁-adrendergic receptor inhibitors (e.g. terazosin, doxazosin, alfuzosin and tamsulosin) and 5-α-reductase inhibitors (e.g. dutasteride and finasteride) or combination therapy using both classes of inhibitors (Kyprianou et al., 2000). “Alpha blockade” with doxazosin increases apoptosis in the stromal compartment of hyperplastic prostatic tissue, but does not decrease the overall re-occurrence rate of BPH. In contrast, finasteride leads to as much as a 20% reduction of prostate growth over the first year without recurrance of significant prostatic growth.

Approximately 50% of all men have histologic evidence of benign prostatic hyperplasia (BPH) by age 60, which increases to 80% of men by age 80 (Berry et al., 1984). Rather than diffuse hyperplastic enlargement of the prostate, BPH development follows a well-characterized and spatially restricted progression of events. This begins with the formation of periurethral and transition zone stromal nodules, followed by convergent epithelial in-growth via a “budding and branching” morphogenesis, and concluding with progressive, diffuse enlargement of these “BPH nodules” (McNeal, 1990) occurs almost exclusively within the transition and periurethral zones of the prostate.

While the pathologic morphology BPH has been described (Mcneal, 1990; Franks, 1954), relatively little is known about the etiology of BPH, except that it requires the presence of functioning testes and correlates closely with aging. Androgens may play only a passive role in the development of BPH (Coffey et al., 1987). Epidemiological studies searching for an association between BPH and diet, smoking, caffeine, frequency of sexual intercourse, medications, or other diseases, including cardiovascular disease, have produced negative results (Guess, 1992).

There are considerable benefits to having information concerning an undesirable medical condition prior to the onset of the condition or following onset of the condition, including during the early onset of the condition. The present invention concern such benefits for benign prostatic hyperplasia (BPH). In specific embodiments, this information may be obtained before or after any physical symptoms are detectable, for example. The information may allow preventive and/or therapeutic intervention to delay onset, inhibit onset, ameliorate at least one symptom of, or eradicate at least one symptom in the individual. In other aspects, it is desirable to determine whether or not a therapy for an individual will be effective. In the event that it is not effective, it would be beneficial to employ alternative treatment and avoid further development of the disease. In specific embodiments, the disease comprises resistance to the therapy. In specific embodiments, this information may be obtained before the treatment is utilized or prior to detectable resistance to the treatment. Regardless, the information may allow preventive and/or therapeutic intervention to delay onset, inhibit onset, ameliorate at least one symptom, or eradicate at least one symptom in the individual.

The present invention concerns particular biological pathways identified with BPH and/or treatment of BPH, and these pathways are predictive of BPH and/or provide therapeutic targets for BPH not previously identified. The pathways may provide targets for BPH particularly for pathways associated with resistance to a treatment thereof, for example. The pathways may be of any kind identified by methods described herein, but in particular embodiments, the pathways include inflammatory pathway, Wnt signaling pathway, extracellular matrix remodeling pathway, cell signaling pathway, cell cycle pathway, or a combination thereof, for example. In specific embodiments, particular polynucleotides having gene products present in one or more of the pathways, or the gene products themselves, are associated with BPH and/or a therapy thereof, such as resistance to BPH therapy. In further specific embodiments, the expression level of one or more particular polynucleotides and/or the expressed product thereof is indicative of a risk for developing BPH or is indicative of having BPH.

In a non-limiting embodiment, the present inventors employed commercially available gene expression chips for an initial analysis of genes having expression upregulated or downregulated compared to a control. More particularly, a plurality of expressed transcripts from one or more prostate cells is probed to a chip having a variety of immobilized nucleic acid sequences of known location. A signal is produced upon hybridization of a probe to a complementary sequence on the chip, and the intensity of the signal is obtained. An increase in a signal may be commensurate with an increased level of a particular transcript. Alternatively, a decrease in a signal may be commensurate with a decreased level of a particular transcript.

In particular, one or more signals are interpreted and evaluated for being indicative of BPH. The present invention provides particular sequences for which upregulation, leading to an increased level of transcripts, and/or for which downregulation, leading to a decreased level of transcripts, provides information concerning BPH, including its presence, its potential for developing, and/or its treatment.

In particular embodiments, the polynucleotides and/or gene products diagnostic or therapeutic for an individual may be obtained from one or more cells. The cells may come from an individual suspected of being susceptible to BPH, an individual suspected of having BPH, an individual that has BPH, an individual with an enlarged prostate, and/or a human male over the age of about 45.

In particular, the present invention employs an RNA expression profile (which may also be referred to as a gene expression profile), to predict a susceptibility to or to predict the potential for developing or to predict the occurrence and/or treatment for BPH. In particular, the polynucleotides may already be expressed in a normal prostate but their levels may change in response to the enlargement of the prostate and/or in response to environmental and/or genetic factors that predispose the individual to developing an enlarged prostate. In specific embodiments, the levels of the polynucleotides become altered prior to detection of a prostate enlarging and/or become altered following detectable enlargement.

Although in particular embodiments the level of gene expression is reflected in mRNA levels, in alternative embodiments, the protein products encoded therefrom are indicative of levels of expression of the mRNA. Standard methods in the art are known to determine protein levels, including by western immunoblot, for example.

In specific embodiments, the level of one or more polynucleotides is upregulated in a big prostate, and there is a linear correlation between prostate size and their expression. In additional embodiments, the level of one or more polynucleotides is downregulated in a big prostate, and there is a linear correlation between between prostate size and their expression. In particular embodiments, the expression level is measured as an RNA or an encoded gene product level.

In specific aspects of the invention, there is as a composition of matter, isolated expressed polynucleotides the levels of which are indicative of a risk for developing benign prostatic hyperplasia (BPH), and/or the presence of BPH. In specific embodiments, there are one or more of the expressed polynucleotides are identified in Table 3, Table 4, Table 5, Table 9, or a combination thereof. The expressed polynucleotides may be comprised on a substrate, such as a microarray chip.

In particular embodiments, the composition further comprises BPH risk factor-evaluating information, BPH risk factor-evaluating reagent, or a combination thereof. In particular aspects, the BPH risk factor-evaluating information comprises statistical information for prostate related to age, race, total PSA level, free PSA, % Free PSA, BPSA, -2proPSA, maximum urine flow rate, AUA SI, BPH impact index, PVR, ultrasound total prostate volume, ultrasound transition zone (TZ) volume, or a combination thereof. In specific embodiments, the BPH risk factor-evaluating reagent comprises a PSA level measuring reagent, a maximum urine flow rate measuring reagent, or both. In particular embodiments, a composition for evaluating a risk for BPH is provided in a kit, in a suitable container.

In particular embodiments, the individual in which resistance is predicted for is a human, although the invention is suitable for any mammal, including dogs, cats, horses, and so forth.

In particular embodiments of the invention, an individual with BPH is treated with an agent that targets one or more of the polynucleotides or encoded gene products of the invention. In specific embodiments, the agent targets one or more members an inflammatory pathway, a Wnt pathway, a cell signaling or cell cycle pathway, or a combination thereof. The agent may be an antibody, a small molecule, or a mixture thereof.

As indicated, the present invention in some embodiments concerns particular biological pathways associated with resistance to treatment for BPH. In specific embodiments, particular polynucleotides having gene products present in one or more of the pathways or the gene products themselves are associated with BPH and/or therapy thereof, such as resistance to BPH therapy In further specific embodiments, the expression level of one or more particular polynucleotides and/or the expressed product thereof is indicative of a risk for developing BPH or is indicative of having BPH.

In particular, the cells from which the RNA and/or protein is derived may come from an individual suspected of being susceptible to BPH, an individual suspected of having BPH, an individual that has BPH, an individual with an enlarged prostate, an individual that will be on a BPH therapy, an individual that is on a BPH therapy, and/or a human male over the age of about 45. In specific embodiments, the individual is going to have BPH therapy or is being subjected to BPH therapy, and in further specific embodiments, the therapy is susceptible for an individual to develop resistance to it.

In particular, the present invention employs an RNA expression profile (which may also be referred to as a gene expression profile), to predict a susceptibility to or to predict the potential for developing or to predict the occurrence and/or treatment. In particular, the polynucleotides may already be expressed in a normal prostate but their levels may change in response to a therapy or in response to the enlargement of the prostate and/or in response to environmental and/or genetic factors that predispose the individual to developing an enlarged prostate or having or becoming resistant to the therapy. In specific embodiments, the levels of the polynucleotides become altered prior to detection of a prostate enlarging and/or become altered following detectable enlargement. In further specific embodiments, the expression profile provides information concerning the risk of an individual to develop resistance to a BPH therapy. In additional specific embodiments, the expression profile provides information concerning the chance of a therapy for being effective in an individual about to be treated for BPH, and/or being treated for BPH. In particular embodiments, the therapy comprises finasteride, tamsulosin, or both.

Although in particular embodiments the level of gene expression is reflected in mRNA levels, in alternative embodiments, the protein products encoded therefrom are indicative of levels of expression of the mRNA. Standard methods in the art are known to determine protein levels, including by western immunoblot, for example.

In certain embodiments of the invention, the methods described herein are employed in conjunction with or as a supplement to additional methods identifying particular polynucleotides and gene products associated with BPH diagnosis or treatment. Furthermore, the particular polynucleotides and gene products identified with methods of the present invention may be subjected to directed methods for additional study, such as with RT-PCR from prostate samples, for example.

In specific embodiments of the invention, prior to or upon early treatment with one or more BPH therapies the kinds of polynucleotides expressed and their levels are measured, such as to identify if the respective therapy will be or is effective or not. They may be measured by any suitable means, such as by microchip analysis, RT-PCR, immunoblot, and so forth. In particular embodiments, these polynucleotides and their encoded gene products are present in one or more pathways, including an inflammatory pathway, a Wnt pathway, an extracellular matrix modeling pathway, a cell cycle pathway, or a cell signaling pathway.

In particular embodiments, the methods and compositions of the invention utilize additional BPH risk factors in evaluating response to BPH therapy. Thus, the present invention further comprises BPH risk factor-evaluating information, BPH risk factor-evaluating reagent, or a combination thereof. In particular aspects, the BPH risk factor-evaluating information comprises statistical information for prostate related to age, race, total PSA level, free PSA, % Free PSA, BPSA, -2proPSA, maximum urine flow rate, American Urological Association (AUA) Symptom Index (SI), BPH impact index, PVR ultrasound total prostate volume, ultrasound transition zone (TZ) volume, or a combination thereof. In specific embodiments, the BPH risk factor-evaluating reagent comprises a PSA level measuring reagent, a maximum urine flow rate measuring reagent, or both. In particular embodiments, a composition for evaluating a risk for BPH is provided in a kit, in a suitable container.

In particular embodiments, the present inventors may employ commercially available gene expression chips for an initial analysis of genes having expression upregulated or downregulated in response to therapy compared to a control. More particularly, a plurality of expressed transcripts from one or more prostate cells is probed to a chip having a variety of immobilized nucleic acid sequences of known location. A signal is produced upon hybridization of a probe to a complementary sequence on the chip, and the intensity of the signal is obtained. An increase in a signal may be commensurate with an increased level of a particular transcript. In particular, one or more signals are interpreted and evaluated for being indicative of BPH. The present invention provides particular sequences for which upregulation, which leads to an increased level of transcripts, and/or for which downregulation, which leads to a decreased level of transcripts, provides information concerning BPH, including its presence, its potential for developing, and/or its treatment.

In particular embodiments, the individual in which a chance of resistance is predicted for is a human, although the invention is suitable for any mammal, including dogs, cats, horses, and so forth.

III. Prostate and Benign Prostate Hyperplasia (BPH)

BPH comprises enlargement of the prostate, and it often occurs in men over the age of about 50. For some individuals, the enlargement may result in compression of the urethra, which sometimes causes lower urinary tract symptoms (LUTS). Although some individuals are asymptomatic, some symptoms may include a weak urinary stream; difficulty initiating urination; frequent urination; and/or frequent awakening at night for urination. In those individuals wherein there is blockage of the urethra as a result of BPH, there may be repeated urinary tract infections, a sudden inability to urinate, and/or gradual bladder and/or kidney damage, for example.

In particular aspects of the invention, an enlarged prostate may be considered to be one that is over about 35 grams in weight, whereas a normal prostate, which may also be referred to herein as a small prostate, is one that is about 20-30 grams. In particular aspects of the invention, a big prostate is one that is over about 70 grams in weight. In a specific embodiment, the size of the prostate may be measured by ultrasound, for example.

BPH may be diagnosed in a variety of ways, although a digital rectal examination (DRE) may be performed in a particular aspect, given that the prostate lies in front of the rectum, by inserting a gloved, lubricated finger into the rectum. In this manner, the prostate will be available for determining by feel whether it is enlarged or comprises lumps or other abnormalities.

Regarding treatment of BPH, medications, nonsurgical procedures that use heat to destroy excess tissue, and/or surgery may be employed. For example, medications work to relax the muscle tissue in the prostate or by reducing the amount of the hormone dihydrotestosterone (DHT). In some embodiments, the enlarged part of the prostate is removed, especially as a long-term solution for patients with BPH. In particular, the enlarged tissue that is pressing against the urethra may be removed, such that the remainder of the prostate tissue and the outside capsule are left intact. However, for individuals with less than severe symptoms, there may be no therapy employed other than monitoring of the progression of symptoms and any complications that may result.

Prior to the present invention, BPH may be diagnosed in a variety of standard ways in the art. For example, a digital rectal examination (DRE) may be performed, given that the prostate lies in front of the rectum, by inserting a gloved, lubricated finger into the rectum. In this manner, the prostate will be available for determining by feel whether it is enlarged or comprises lumps or other abnormalities.

Also prior to the present invention, current treatment of BPH may comprise medications, nonsurgical procedures that use heat to destroy excess tissue, and/or surgery, for example. Medications may work to relax the muscle tissue in the prostate or by reducing the amount of the hormone dihydrotestosterone (DHT). In some embodiments, the enlarged part of the prostate is removed, especially as a long-term solution for patients with BPH. In particular, the enlarged tissue that is pressing against the urethra may be removed, such that the remainder of the prostate tissue and the outside capsule are left intact. However, for individuals with less than severe symptoms, there may be no therapy employed other than monitoring of the progression of symptoms and any complications that may result.

Exemplary treatment for BPH is described in U.S. Pat. No. 6,733,779, directed to oral administration of cis-retinoic acid. An alternative treatment is described in U.S. Pat. No. 6,410,554, which regards alpha-1a antagonist and an endothelin antagonist. Heterocyclic substituted piperazines of a specific formula are described for treatment of BPH in U.S. Pat. No. 6,384,035. U.S. Pat. No. 6,048,888 describes (1) an effective BPH treating amount of melatonin; and optionally (2) antiandrogens, antiestrogens, growth hormones and/or inhibitors of prostatal testosterone reductase; and/or (3) oxazepam or other melatonin receptor profile modifier for treating BPH.

It is known that both BPH and prostate cancer can exist together, and, therefore, the embodiments of the present invention are suitable applicable to the diagnosis and/or treatment of prostate cancer.

IV. RNA Expression Profiling and RNA Expression Profiles

In specific embodiments, RNAs are indicative of a susceptibility to developing BPH, a risk for developing BPH, or the presence of BPH. The RNAs that are associated with BPH may be any expressed RNA or RNAs that assist in the evaluation of BPH. The expression profile may indicate those individuals that will develop BPH or that have BPH.

In specific embodiments, there may be one or more expressed genes identified in Table 3, Table 4, Table 5 and/or Table 9 as associated with BPH and therefore is useful for diagnosing its presence or susceptibility thereto in an individual. In additional embodiments, there may be combinations of expressed genes identified in Table 3, Table 4, Table 5 and/or Table 9 as being indicative of BPH. There may be combinations of two expressed genes, three expressed genes, four expressed genes, or five or more expressed genes, for example.

In specific embodiments, there may be one or more expressed genes identified in Table 7 as associated with benign prostate hyperplasia and therefore is useful for diagnosing its presence or susceptibility thereto in an individual. In additional embodiments, there may be combinations of expressed genes identified in Table 7 as being indicative of benign prostate and/or being indicative of response to a particular BPH therapy. There may be combinations of two expressed genes, three expressed genes, four expressed genes, or five or more expressed genes, for example.

A skilled artisan recognizes that the relevance of the expressed polynucleotides indicative of BPH and/or its response to treatment may be confirmed by routine methods in the art other than expression on a profile of polynucleotides. For example, a change in expression of a polynucleotide suspected of being associated with BPH, such as identified during standard microchip analysis, may be confirmed by other means, such as by directed quantitative RT-PCR, for example. Also for example, demonstration that upregulation or downregulation of a particular polynucleotide confers resistance to a BPH therapy may employ genetic engineering. Furthermore, inhibitors of the appropriate signaling pathways and/or siRNA knock-down studies to reduce the levels of potential ER accessory proteins or signaling molecules may be utilized. The development of small molecule inhibitors to resistance genes are ideal targets for drug development using rational drug design, in vitro chemical library screening on either a small or large scale, and high-content target-based cellular assays.

In specific embodiments, an expressed finasteride-resistant and/or tamsulosin-resistant and/or finasteride-sensitive and/or tamsulosin-sensitive gene is assessed in an in vivo model system. For example, vectors comprising the expressed gene in question may be delivered to a BPH mouse model. Following this, the mice are administered a BPH drug for a period of time. If the gene is related to resistance to finasteride and/or tamsulosin, then the prostate size of the transformed mice should increase. In vitro methods may also be employed to confirm association of a particular gene with finasteride and/or tamsulosin resistance. For example, the ability of prostate cells to proliferate may be utilized in BPH therapy resistance.

V. Collection of Samples

In aspects of the invention, samples are obtained from an individual for subjecting to the methods and compositions described herein. The samples may come from an individual suspected of having BPH, from an individual suspected of developing BPH, and/or from an individual that is susceptible to developing BPH. An individual suspected of being susceptible to BPH may in specific embodiments be a human male over the age of about 40.

Any suitable methods for obtaining the samples are within the scope of the invention, and exemplary methods include biopsy, including extraction of part or all of the prostate. In further specific embodiments, fine needle aspirates are obtained via a biopsy procedure. Samples may additionally or alternatively be collected via prostate massage, such as, for example, by producing prostatic fluid; from blood; from urine; from serum; seminal fluid; or a combination thereof. In a specific embodiment, the biopsy that is obtained substantially lacks any prostatic cancer cells; as such, the sample may be referred to as a non-cancerous sample. However, in alternative embodiments, the sample may comprise one or more prostate cancer cells.

One or more cells of the samples may be isolated and used to obtain nucleic acid, such as DNA and/or RNA, from said cell(s). In addition or alternatively, protein may be obtained from the cells. In specific embodiments of the invention, the isolation of one or more cells may be performed by microdissection, such as, but not limited to, laser capture microdissection (LCM) or laser microdissection (LMD). In particular embodiments, RNA from the cells of the samples are obtained for analysis, such as mRNA transcripts. The levels and/or activities of the mRNA(s) may be assayed directly or indirectly, or they may be amplified in whole or in part prior to detection.

VI. Specific Embodiments of Diagnostic/Therapeutic Polynucleotides

In particular embodiments of the present invention, one or more polynucleotides provide information concerning the susceptibility of an individual to develop BPH or the presence of BPH in an individual, and/or the ability to have or develop resistance to a BPH therapy. In particular embodiments, the information provided by the polynucleotides derives from their expression level, and this level may be interpreted from RNA or from expressed protein, or both. The level of a particular polynucleotide may be higher compared to a control, may be lower compared to a control, or there may be a plurality of polynucleotides some of which are higher and some of which are lower than there respective counterparts in a control.

A skilled artisan recognizes how to identify predictive/therapeutic polynucleotides based on description provided herein and the standard reagents and methods available in the art. Thus, in particular aspects of the invention, one or more polynucleotides associated with BPH and/or resistance or effectiveness to BPH therapy may be provided herein or determined by the skilled artisan based on the well-described methods provided herein.

In some embodiments, the polynucleotides predictive of BPH development or presence are known to be associated with one or more particular pathways in a cell. In specific embodiments, the polynucleotide encodes a gene product present in an inflammatory pathway, a Wnt signaling pathway, a cell cycle pathway, a cell signal pathway, an extracellular matrix remodeling pathway, and so forth. These pathways and gene products present therein provide useful targets for BPH. For example, the polynucleotides may be targeted with antisense RNA or siRNA, or the encoded products from the polynucleotides may be targeted with antibodies or small molecules, for example; furthermore, a combination thereof may be employed. The polynucleotides and/or their encoded products may also be targeted with, for example, an engineered receptor chimera, such as one having an Ig with a receptor binding domain. Alternatively, a ligand having a moiety that crosslinks to another molecule may be employed.

Exemplary and particular polynucleotides associated with BPH are contemplated herein, and their non-limiting functions are provided below. Analogous information for polynucleotides and their encoded products not provided herein may be obtained from the literature and/or from the World Wide Web, such as the websites and/or links therein for the National Center for Biotechnology Information and/or the website for Applied Biosystems, Inc, for example. Alternatively, the function of a gene product in question may be inferred and confirmed by one or more particular domains identified in the sequence, based on comparison to sequences having a known function, and this function may be confirmed by an appropriate study. For example, if a sequence of unknown function was identified by methods described herein, the sequence could be submitted to a database for comparison with other sequences for identification of one or more characteristic domains. As an example, a sequence of unknown function is submitted to the Blast function of the Genbank®® database from the website of the National Center for Biotechnology Information, and the sequence identifies a kinase domain. Suitable studies to determine if the gene product is a kinase are then employed, such as those standard in the art.

The following are specific embodiments of sequences associated with BPH diagnosis and/or therapy and/or resistance to a BPH therapy, and they are provided in no particular order.

Frizzled 10

Members of the frizzled family encode 7-transmembrane domain proteins that are receptors for the Wingless type MMTV integration site family of signaling proteins, in specific embodiments. In other specific embodiments, most frizzled receptors are coupled to the beta-catenin canonical signaling pathway.

Sprouty 2

This polynucleotide encodes a protein belonging to the sprouty family. In one aspect of the invention, the encoded protein comprises a carboxyl-terminal cysteine-rich domain essential for the inhibitory activity on receptor tyrosine kinase signaling proteins and is required for growth factor stimulated translocation of the protein to membrane ruffles. In primary dermal endothelial cells, this polynucleotide is transiently upregulated in response to fibroblast growth factor two. This protein is indirectly involved in the non-cell autonomous inhibitory effect on fibroblast growth factor two signaling. The protein interacts with Cas-Br-M (murine) ectropic retroviral transforming sequence, and it can function as a bimodal regulator of epidermal growth factor receptor/mitogen-activated protein kinase signaling.

Major Histocompatibility Complex, Class II, DP Alpha 1 (HLA-DPA1)

HLA-DPA1 is a HLA class II alpha chain paralog. In an embodiment of the invention, this class II molecule is a heterodimer comprising an alpha (DPA) and a beta (DPB) chain, both anchored in the membrane. In particular embodiments, it plays a role in the immune system by presenting peptides derived from extracellular proteins. Class II molecules are expressed in antigen presenting cells (APC: B lymphocytes, dendritic cells, macrophages, in exemplary embodiments). The alpha chain is approximately 33-35 kDa and its polynucleotide comprises 5 exons. Exon one encodes the leader peptide, exons 2 and 3 encode the two extracellular domains, and exon 4 encodes the transmembrane domain and the cytoplasmic tail. Within the DP molecule, both the alpha chain and the beta chain contain the polymorphisms specifying the peptide binding specificities, resulting in up to 4 different molecules.

Peptidylprolyl Isomerase C (Cyclophilin C)

Peptidylprolyl isomerase C (cyclophilin C) may also be referred to as PPIase C; parvulin; rotamase C; or cyclophilin C. The protein encoded by this polynucleotide is a member of the peptidyl-prolyl cis-trans isomerase (PPIase) family. PPIases catalyze the cis-trans isomerization of proline imidic peptide bonds in oligopeptides and accelerate the folding of proteins. Similar to other PPIases, this protein can bind immunosuppressant cyclosporin A.

CXCL9: Chemokine (C-X-C Motif) Ligand 9

The function of gene product encoded by this polynucleotide has not been specifically defined; however, it is thought to be involved in T cell trafficking. This gene has been localized to 4q21 with INP10, which is also a member of the chemokine family of cytokines.

CXCL10; Chemokine (C-X-C Motif) Ligand 10

CXCL10 may also be referred to as gamma IP10, interferon-inducible cytokine IP-10, protein 10 from interferon (gamma)-induced cell line, or small inducible cytokine subfamily B (Cys-X-Cys), member 10.

In particular embodiments,this polynucleotide encodes the interferon (gamma)-induced protein of 10 kDa, a chemokine of the CXC subfamily that is one of the ligands for the receptor CXCR3. The binding of this protein to CXCR3 causes pleiotropic effects, including stimulation of monocytes, natural killer and T-cell migration, and modulation of adhesion molecule expression, in specific embodiments.

CXCL11: Chemokine (C-X-C Motif) Ligand 11

CXCL11 may also be referred to as small inducible cytokine subfamily B (Cys-X-Cys), member 11 or small inducible cytokine subfamily B (Cys-X-Cys), member 9B.

Chemokines are a group of small (approximately 8 to 14 kD), mostly basic, structurally related molecules that regulate cell trafficking of various types of leukocytes through interactions with a subset of 7-transmembrane, G protein-coupled receptors. Chemokines also play fundamental roles in the development, homeostasis, and function of the immune system, and they have effects on cells of the central nervous system as well as on endothelial cells involved in angiogenesis or angiostasis. Chemokines are divided into 2 major subfamilies, CXC and CC. This polynucleotide is a CXC member of the chemokine superfamily. Its encoded protein induces a chemotactic response in activated T-cells and is the dominant ligand for CXC receptor-3. The gene encoding this protein contains 4 exons and at least three polyadenylation signals which might reflect cell-specific regulation of expression. IFN-gamma is a potent inducer of transcription of this gene.

Activating Transcription Factor 3

Activating transcription factor 3 (ATF3) is a member of the mammalian activation transcription factor/cAMP responsive element-binding (CREB) protein family of transcription factors. It encodes a protein with a calculated molecular mass of 22 kD. ATF3 represses rather than activates transcription from promoters with ATF binding elements. An alternatively spliced form of ATF3 (ATF3 delta Zip) encodes a truncated form ATF3 protein lacking the leucine zipper protein-dimerization motif and does not bind to DNA. In contrast to ATF3, ATF3 delta Zip stimulates transcription, in specific embodiments by sequestering inhibitory co-factors away from the promoter. It is possible that alternative splicing of the ATF3 gene may be physiologically important in the regulation of target genes.

BBS4: Bardet-Biedl Syndrome 4

This polynucleotide encodes a protein that comprises tetratricopeptide repeats (TPR), similar to O-linked N-acetyglucosamine transferase. Mutations in this polynucleotide have been observed in patients with Bardet-Bied1 syndrome type 4. The encoded protein in particular embodiments plays a role in pigmentary retinopathy, obesity, polydactyly, renal malformation and mental retardation.

TM4SF7: Transmembrane 4 Superfamily Member 7

Transmembrane 4 superfamily member 7 may also be referred to as TM4SF7, tetraspanin 4, novel antigen 2, or tetraspan TM4SF. The protein encoded by this polynucleotide is a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth and motility. This encoded protein is a cell surface glycoprotein and is similar in sequence to its family member CD53 antigen. It is known to complex with integrins and other transmembrane 4 superfamily proteins.

EPH Receptor A4

The EPH receptor A4 may also be referred to as ephrin type-A receptor 4; TYRO1 protein tyrosine kinase; or tyrosine-protein kinase receptor SEK. This polynucleotide belongs to the ephrin receptor subfamily of the protein-tyrosine kinase family. EPH and EPH-related receptors have been implicated in mediating developmental events, particularly in the nervous system. Receptors in the EPH subfamily typically have a single kinase domain and an extracellular region containing a Cys-rich domain and 2 fibronectin type III repeats. The ephrin receptors are divided into 2 groups based on the similarity of their extracellular domain sequences and their affinities for binding ephrin-A and ephrin-B ligands.

Ras Protein-Specific Guanine Nucleotide-Releasing Factor 2

RAS GTPases cycle between an inactive GDP-bound state and an active GTP-bound state. Guanine-nucleotide exchange factors (GEFs), such as RASGRFs, stimulate the conversion of the GDP-bound form into the active form.

HS3ST3B1: Heparan Sulfate (Glucosamine) 3-O-Sulfotransferase 3B1

Heparan sulfate biosynthetic enzymes are key components in generating a myriad of distinct heparan sulfate fine structures that carry out multiple biologic activities. The enzyme encoded by this polynucleotide is a member of the heparan sulfate biosynthetic enzyme family. It is a type II integral membrane protein and possesses heparan sulfate glucosaminyl 3-O-sulfotransferase activity. The sulfotransferase domain of this enzyme is highly similar to the same domain of heparan sulfate D-glucosaminyl 3-O-sulfotransferase 3A1, and these two enzymes sulfate an identical disaccharide. This polynucleotide is widely expressed.

Beta-Catenin

Beta-catenin is an adherens junction protein, in some aspects of the invention. Adherens junctions (AJs; also called the zonula adherens) are critical for the establishment and maintenance of epithelial layers, such as those lining organ surfaces. AJs mediate adhesion between cells, communicate a signal that neighboring cells are present, and anchor the actin cytoskeleton. In serving these roles, AJs regulate normal cell growth and behavior. At several stages of embryogenesis, wound healing, and tumor cell metastasis, cells form and leave epithelia. This process, which involves the disruption and reestablishment of epithelial cell-cell contacts, in specific embodiments is regulated by the disassembly and assembly of AJs. AJs may also function in the transmission of the ‘contact inhibition’ signal, which instructs cells to stop dividing once an epithelial sheet is complete.

KLK4

Kallikreins are a subgroup of serine proteases having diverse physiological functions. Growing evidence suggests that many kallikreins are implicated in carcinogenesis and some have potential as novel cancer and other disease biomarkers. This gene is one of the fifteen kallikrein subfamily members located in a cluster on chromosome 19. In some tissues its expression is hormonally regulated.

MMP12

Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. In specific embodiments, the protein encoded by this gene is cleaved at both ends to yield the active enzyme. The enzyme degrades soluble and insoluble elastin. It may play a role in aneurysm formation and studies in mice suggest a role in the development of emphysema. The gene is part of a cluster of MMP genes which localize to chromosome 11q22.3.

MMP9

The enzyme encoded by this polynucleotide degrades type IV and V collagens. Studies in rhesus monkeys suggest that the enzyme is involved in IL-8-induced mobilization of hematopoietic progenitor cells from bone marrow, and murine studies suggest a role in tumor-associated tissue remodeling.

ORM1

This polynucleotide encodes a key acute phase plasma protein. Because of its increase due to acute inflammation, this protein is classified as an acute-phase reactant. In specific aspects of the invention, this protein is involved in aspects of immunosuppression.

PRV1

NB1, a glycosyl-phosphatidylinositol (GPI)-linked N-glycosylated cell surface glycoprotein, was first described in a case of neonatal alloimmune neutropenia.

SFRP4

Secreted frizzled-related protein 4 (SFRP4) is a member of the SFRP family that contains a cysteine-rich domain homologous to the putative Wnt-binding site of Frizzled proteins. SFRPs act as soluble modulators of Wnt signaling. The expression of SFRP4 in ventricular myocardium correlates with apoptosis related gene expression.

VEGF

Vascular endothelial growth factor is a mitogen primarily for vascular endothelial cells. It is, however, structurally related to platelet-derived growth factor.

WIF1

WNT proteins are extracellular signaling molecules involved in the control of embryonic development. This gene encodes a secreted protein, which binds WNT proteins and inhibits their activities. This protein contains a WNT inhibitory factor (WIF) domain and 5 epidermal growth factor (EGF)-like domains.

DKK3

Dickkopfs (Dkks) are secreted developmental regulators comprised of two cysteine-rich domains. In specific aspects, DKK3 is an inhibitor of WNT signaling and in further aspects plays a role in mediating interactions between epithelial and mesenchymal cells.

Contactin

Contactin is a neuronal cell surface glycoprotein that plays a role in cell adhesion.

VII. Pharmaceutical Preparations

In particular embodiments, an agent that targets one or more BPH-diagnostic and/or BPH-therapeutic polynucleotides or encoded products or an agent that targets one of the receptors thereof (such as CXCR3) is employed in a pharmaceutical composition for the treatment of BPH. These agents may be an antibody, a small molecule, antisense RNA, and so forth, for example. In specific embodiments, the pharmaceutical composition may be employed in the event of resistance having been identified or developed in an individual on BPH therapy or an alternative therapy is employed following identification of resistance to the present therapy.

Pharmaceutical compositions of the present invention comprise an effective amount of one or more BPH therapeutic agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one BPH therapeutic agent or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The BPH therapeutic agent may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The BPH therapeutic agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include one or more BPH therapeutic agents, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the BPH therapeutic agent may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according tot he response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the BPH therapeutic agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, BPH therapeutic agent may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound BPH therapeutic agent may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### EXAMPLES

The following examples are offered by way of example and are not intended to limit the scope of the invention in any manner.

### Example 1

### Exemplary Methods and Materials for Example 2

In particular embodiments of the present invention, a prostate is examined for its size, and the gene expression of one or more cells therein is assayed. Alternatively, gene expression in the prostate may be assayed in the absence of determining the size of the prostate. The following table represents the sample size number for exemplary studies regarding changes in gene expression in big vs. small prostates.

TABLE 1
Changes in gene expression in big vs. small prostates

| | Mean Prostate Size | Sample Size |
|---|---|---|
| Group | (g) | Chip |
| BIG | 84 ± 13.7 | 5 |
| SMALL | 23.6 ± 4.5 | 2 |

Through an ongoing IRB-approved protocol (H-1158), zone-specific tissue samples were harvested from patients undergoing radical prostatectomy for clinically localized prostate cancer and were frozen in liquid nitrogen for later analysis. In addition to prostate cancer tissue samples, samples from the transition zone from these patients have also been harvested. Since patients with prostate cancer undergoing prostate cancer surgery often also have concomitant BPH, the present inventors have collected a large number of samples from patients, ranging from those with very small prostates (<25 cc) not exhibiting pathologic BPH changes in the prostatic transition zone, to those with very big prostates (>100 cc), exhibiting severe pathologic BPH changes in the prostatic transition zone. Frozen transition zone (TZ) samples were selected from both groups of patients, isolated mRNA and performed cDNA microarray analysis using Affymetrix U133 Plus 2.0 gene arrays to determine differential gene expression between these protate samples. These studies have lead to the identification of new genes and genetic pathways associated with severe BPH-related prostate growth.

### Example 2

### Gene Expression in Benign Prostatic Hyperplasia

After standard quality control analysis of samples and extracted mRNA, 7 samples subjected to microarray chip hybridization (in conjunction with the Baylor College of Medicine Microarray Core Facility) were compared, 5 from big BPH-affected prostates, and 2 from small non-affected prostates (Table 2).

TABLE 2
Big prostate (B; mean = 84 g ± 13.7 g) and small
prostate (S; mean = 23.6 g ± 4.5 g)
samples used in Affymetrix U133 Plus 2.0 microarray analysis.
Big vs Small Prostate
Microarray Analyses

| Group | Sample ID | Req ID |
|---|---|---|
| B | 2476 | 453 |
| B | 1756 | 1039 |
| B | 2791 | 1039 |
| B | 4894 | 1039 |
| B | 4897 | 1039 |
| S | 2208 | 453 |
| S | 4918 | 1039 |

Through this analysis, a novel inflammatory pathway was identified, including the genes listed in Table 3, that were associated with a 2-4 fold increase in the levels of expression in the development of BPH-related prostate enlargement. These genes are all members of an interferon gamma-inducible inflammatory pathway also implicated in the regulation of angiogenesis and T cell chemotaxis.

TABLE 3
Inflammatory genes upregulated by 2-4 fold in big prostate tissue

| | | Gene |
|---|---|---|
| Fold Δ | Gene Title | Function |
| 3.14 | chemokine (C—X—C motif) ligand 11 (also referred to as CXCL11, IP9, H174, | (I-TAC) |
| | IP-9, b-R1, I-TAC, SCYB11, or SCYB9B; SEQ ID NO: 15 nucleic acid | Inflammation/T |
| | comprises Genbank ® Acc. No. NM_005409; SEQ ID NO: 16 protein | cell |
| | comprises Genbank ® Acc. No. NP_005400.1) | chemotaxis/IF |
| | | Nγ inducible |
| 3.10 | chemokine (C—X—C motif) ligand 10 (also referred to as CXCL10, C7, IFI10, | (IP-10) |
| | INP10, IP-10, crg-2, mob-1, SCYB10, or gIP-10; SEQ ID NO: 13 nucleic acid | Inflammation/T |
| | comprises Genbank ® Acc. No. NM_001565; SEQ ID NO: 14 protein | cell |
| | comprises Genbank ® Acc. No. NP_001556.1) | chemotaxis/IF |
| | | Nγ inducible |
| 2.48 | chemokine (C—X—C motif) ligand 9 (also referred to as CXCL9, CMK, MIG, | (MIG) |
| | Humig, SCYB9, or crg-10; SEQ ID NO: 11 nucleic acid comprises | Inflammation/T |
| | Genbank ® Acc. No. NM_002416; SEQ ID NO: 12 protein comprises Gen | cell |
| | Bank ® Acc. No. NP_002407.1) | chemotaxis/IF |
| | | Nγ inducible |
| 2.28 | peptidylprolyl isomerase C (cyclophilin C) | IFNγ inducible/ |
| | | CsA binding/ |
| | | Ca²⁺ signaling |
| 2.00 | peptidylprolyl isomerase C (cyclophilin C) (also referred to as PPIC, CYPC, | IFNγ inducible/ |
| | or MGC3673; SEQ ID NO: 9 nucleic acid comprises Genbank ® Acc. No. | CsA binding/ |
| | NM_000943; SEQ ID NO: 10 protein comprises Genbank ® Acc. No. | Ca²⁺ signaling |
| | NP_000934.1) | |
| 1.95 | major histocompatibility complex, class II, DP alpha 1 (also referred to as | Antigen |
| | HLA-DPA1, HLA-D, HLADP, HLASB, HLA-DRA, or HLA-DP1A; SEQ ID | presentation to |
| | NO: 7 nucleic acid comprises Genbank ® Acc. No. NM_033554; SEQ ID | T cells |
| | NO: 8 protein comprises Genbank ® Acc. No. NP_291032.2) | |

In particular embodiments, additional studies were employed to verify the sequences identified in Table 3. More specifically, quantitative RT-PCR was performed in a greater number of samples, and the same genes identified in Table 3 were confirmed to have the following upregulation fold change upon comparison of big prostates to small prostates: chemokine (C-X-C motif) ligand 11 (2.10 fold change); chemokine (C-X-C motif) ligand 10 (2.03 fold change); chemokine (C-X-C motif) ligand 9 (2.38 fold change); cyclophilin C (2.18 fold change); and MHC, class II, DP alpha 1 (2.44 fold change).

In specific embodiments, this panel of genes alone and in combination are useful as diagnostic and/or therapeutic markers of BPH, as well as these members grouped by their participation in a specific inflammatory pathway as a therapeutic target pathway for the treatment of BPH.

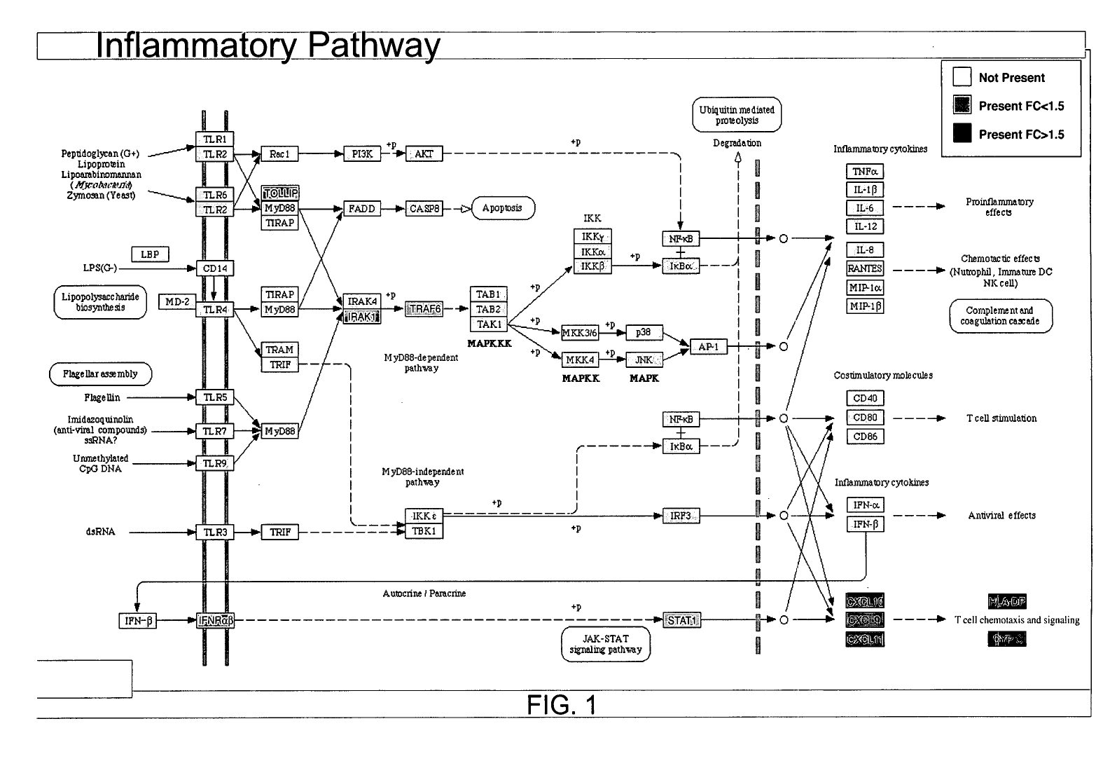

Other inflammatory pathway genes not listed in Table 3 may be indicative of the susceptibility of BPH and/or presence thereof, and an exemplary pathway schematic is provided in FIG. 1.

In additional embodiments of the invention, an additional novel pathway was identified, the Wnt pathway, including the genes listed in Table 4, that were associated with changes in the level of expression in the development of BPH or related effects thereof, such as BPH-related prostate enlargement. These genes are all associated with the Wnt pathway and have not been previously associated with BPH.

TABLE 4
Wnt Pathway Genes (Big vs. Small)

| | | |
|---|---|---|
| 2.04 | dickkopf homolog 3 (also referred to as DKK3 and REIC; | Wnt inhibitor/tumor invasion/MMP |
| | SEQ ID NO: 5 nucleic acid comprises Genbank ® Acc. No. NM_015881; | expression |
| | SEQ ID NO: 6 protein comprises Genbank ® Acc. No. NP_056965.3) | |
| 1.83 | sprouty homolog 2 (Drosophila) (also referred to as | MAPK pathway inhibitor |
| | SPRY2 hSPRY2, or MGC23039; SEQ ID NO: 3 nucleic acid comprises | |
| | Genbank ® Acc. No. NM_005842; SEQ ID NO: 4 protein | |
| | comprises Genbank ® Acc. No. NP_005833.1) | |
| 1.48 | frizzled homolog 10 (Drosophila) (also referred to | Wnt receptor; G protein coupled |
| | as FZD10, FzE7, FZ-10, or hFz10; SEQ ID NO: 1 nucleic acid | receptor |
| | comprises Genbank ® Acc. No. NM_007197; SEQ ID NO: 2 | |
| | protein comprises Genbank ® Acc. No. NP_009128) | |
| 1.33 | Beta catenin (also referred to as CTNNB1, catenin (cadherin- | |
| | associated protein), beta 1, 88 kDa or CTNNB; SEQ ID NO: 37 nucleic acid | |
| | comprises Genbank ® Acc. No. NM_001904; SEQ ID NO: 38 protein | |
| | comprises Genbank ® Acc. No. NP_001895) | |

In further studies, these particular genes were assayed using quantitative RT-PCR, and were identified as having 2.11-fold change, 1.92 fold change, and 1.78 fold change, respectively.

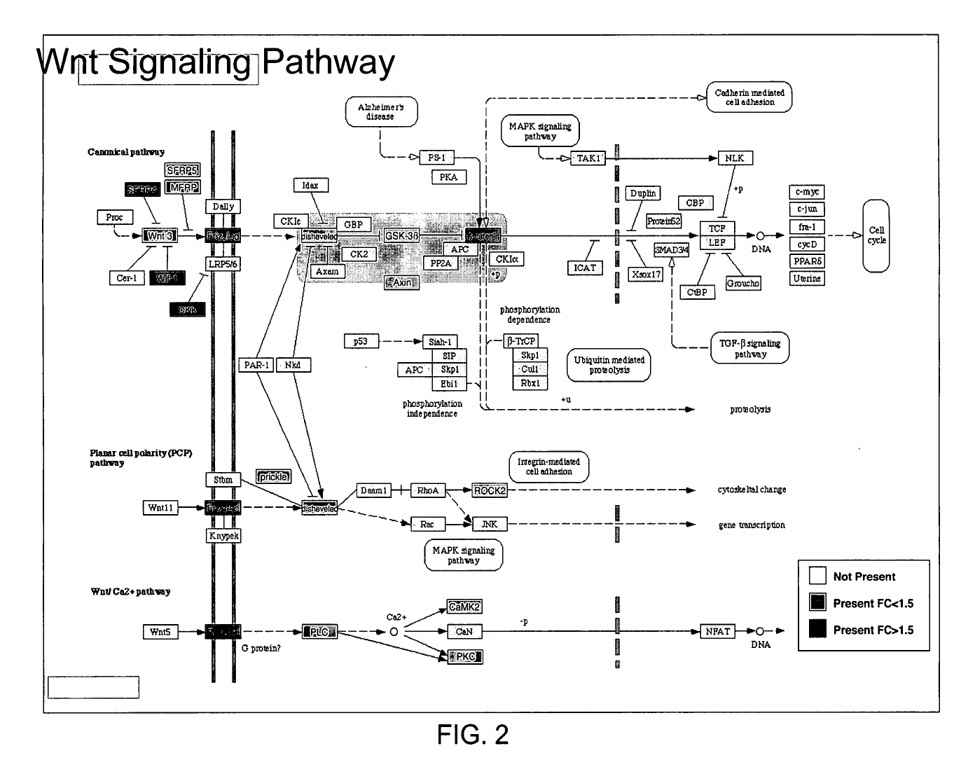

Other Wnt pathway genes not listed in Table 4 may be indicative of the susceptibility of BPH and/or presence thereof, and an exemplary pathway schematic is provided in FIG. 2.

In other embodiments of the invention, another novel pathway was identified, including the genes listed in Table 5, that were associated with changes in the level of expression in the development of BPH or related effects thereof, such as BPH-related prostate enlargement. These genes are all associated with several cell signalling and cell cycle regulatory pathways that may have previously been implicated in prostate cancer progression but have not been described in connection with BPH progression.

TABLE 5
Cell signaling and cell cycle pathway genes in expression level in big prostate
tissue compared to small prostate tissue

| Fold Change | Gene Title | Gene Function |
|---|---|---|
| 3.88 | contactin 1 (nucleic acid in Genbank ® Acc. No. Z21488 (SEQ ID NO: 41); | Extracellular matrix |
| | protein in Genbank ® Acc No. CAA79696; SEQ ID NO: 42)) | |
| 2.91 | heparan sulfate (glucosamine) 3-O-sulfotransferase 3B1 (also referred to | Growth factor induction |
| | as HS3ST3B1, 30ST3B1, or 3OST3B1; SEQ ID NO: 35 nucleic acid | Wnt, hedgehog, FGF |
| | comprises Genbank ® Acc. No. NM_006041; | |
| | SEQ ID NO: 36 protein comprises Genbank ® Acc. No. NP_006032) | |
| 2.8 | Dermatopontin (nucleic acid in Genbank ® Acc. No. NM_001937 | Regulator of TGF-β |
| | (SEQ ID NO: 39); protein | Signaling |
| | in Genbank ® Acc. No. NP_001928; SEQ ID NO: 40) | |
| 2.16 | Fas apoptotic inhibitory molecule 2 (also referred to as FAIM2, LFG, | Apoptosis inhibition |
| | NGP35, NMP35, KIAA0950, lifeguard, or | |
| | neural membrane protein 35; SEQ ID NO: 33 | |
| | nucleic acid comprises Genbank ® Acc. No. NM_012306; SEQ ID NO: 34 | |
| | protein comprises Genbank ® Acc. No. NP_036438) | |
| 2.16 | Ras protein-specific guanine nucleotide-releasing factor 2 (also referred | Signaling in Ras and Erk pathway |
| | to as RASGRF2, GRF2, or RAS-GRF2; SEQ ID NO: 31 nucleic acid | |
| | comprises Genbank ® Acc. No. NM_006909; SEQ ID NO: 32 | |
| | comprises Genbank ® Acc. No. NP_008840) | |
| 2.16 | EphA4 (also referred to as SEK, HEK8, or TYRO1; | tyr kinase receptor/Jak/STAT |
| | SEQ ID NO: 29 nucleic acid comprises Genbank ® Acc. No. NM_004438; | signaling |
| | SEQ ID NO: 30 comprises Genbank ® Acc. No. NP_004429) | |
| 1.95 | transmembrane 4 superfamily member 7 (also referred to as TM4SF7, | Role in tumor metastasis/activatic |
| | NAG-2, TSPAN-4, or TETRASPAN; SEQ ID NO: 27 nucleic acid | of MMPs |
| | comprises Genbank ® Acc. No. NM_003271; SEQ ID NO: 28 comprises | |
| | Genbank ® Acc. No. NP_003262) | |
| −2.22 | Bardet-Biedl syndrome 4 (also referred to as BBS4; SEQ ID NO: 25 nucleic acid | cell cycle regulation and apoptosi |
| | comprises Genbank ® Acc. No. NM_033028; SEQ ID NO: 26 protein | |
| | comprises Genbank ® Acc. No. NP_149017) | |
| −2.31 | activating transcription factor 3 (also referred to as ATF3, | CREB family represses p53 |
| | ATF3deltaZip2, ATF3deltaZip3, or ATF3deltaZip2c; SEQ ID NO: 23 | and MMP-2 exp |
| | nucleic acid comprises Genbank ® Acc. No. AB078026; SEQ ID NO: 24 | |
| | protein comprises BAC00495) | |
| −2.95 | nuclear receptor subfamily 4, group A, member 3 (also referred to | Signaling maybe associates |
| | as NR3C2, MR, MCR, or MLR; SEQ ID NO: 21 nucleic acid | with GDF family |
| | comprises Genbank ® Acc. No. NM_000901; SEQ ID NO: 22 protein | |
| | comprises Genbank ® Acc. No. NP_000892 | |
| −3.01 | SNF1-like kinase (also referred to as SNF1LK, MSK, or SIK; | Possible role in G2/M cell |
| | SEQ ID NO: 19 nucleic acid comprises Genbank ® Acc. No. NM_173354; | cycle progression |
| | SEQ ID NO: 20 protein comprises Genbank ® Acc. No. NP_775490) | |
| −3.14 | SH2 domain protein 1A, Duncan's disease (also referred to as | X-linked lymphoproliferative |
| | LYP, SAP, XLP, DSHP, EBVS, IMD5, XLPD, MTCP1 or | syndrome |
| | SH2D1A; may also be referred to as SLAM (signaling lymphocyte | |
| | activation molecule) associated protein; SEQ ID NO: 17 nucleic acid | |
| | comprises Genbank ® Acc. No. NM_002351; SEQ ID NO: 18 protein | |
| | comprises Genbank ® Acc. No. NP_002342.1) | |

In particular embodiments, additional studies were employed to verify the sequences identified in Table 5. More specifically, quantitative RT-PCR was performed in a greater number of samples, and the same genes identified in Table 5 were confirmed to have the corresponding upregulation or downregulation fold change upon comparison of big prostates to small prostates. As an example, upon quantititative RT-PCR, heparan sulfate (flucosamine) 3-O-sulfotransferase 3B1 had a 2.38 fold change.

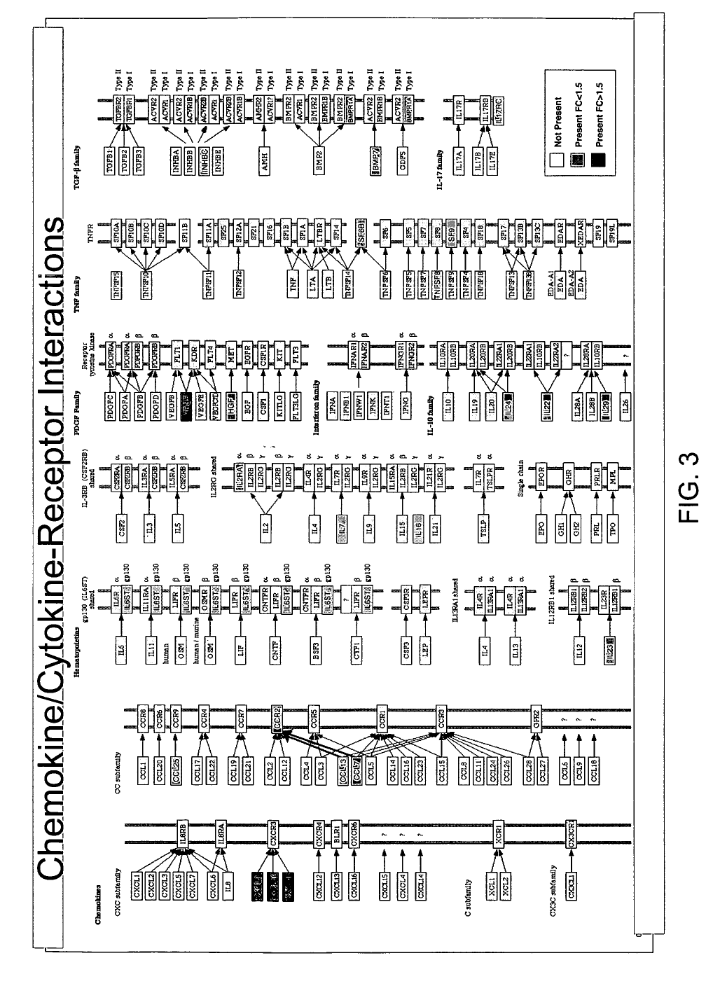

Other cell signaling and cell cycle pathway genes may be indicative of the susceptibility of BPH and/or presence thereof in addition to those identified in Table 5, and an exemplary pathway schematic is provided in FIG. 3.

In specific embodiments of the present invention, these genes alone or in combination are useful as diagnostic and/or therapeutic markers of BPH. Furthermore, these members grouped by their participation in inflammatory, Wnt signaling, cell signaling and cell cycle regulation pathways provide useful therapeutic targets for the prevention and/or treatment of BPH.

### Example 3

### Exemplary Methods and Materials for Example 4

In non-limiting exemplary embodiments of the invention, a randomized, open-label, single-center study was performed to determine the spatio-temporal changes in gene expression within the prostate of patients who receive 0.4 mg of Flomax® (tamsulosin) capsules daily, 5 mg of Proscar® (finasteride) tablets daily, or both for either 6, 4, or 2 weeks prior to radical prostatectomy. Tissue samples from radical prostatectomy specimens were obtained under an ongoing IRB-approved protocol (H-1158). Zone-specific tissue samples from patients undergoing radical prostatectomy for clinically localized prostate cancer were harvested and frozen in liquid nitrogen for later analysis. In addition to prostate cancer tissue samples, samples from the transition zone from these patients have also been harvested.

Participants were randomly allocated to one of three drug treatment arms, after being categorized into one of three groups according to the duration from enrollment date to scheduled surgery date. BPH related symptoms were assessed using self-administered BPH symptom questionnaires at day of enrollment and every 2 weeks (no clinic visit required) until surgery. Medication compliance is assessed at the time of radical prostatectomy.

Exemplary Eligibility Criteria:

Participants meeting the following criteria were eligible for randomization:

1. Male patients (50 years of age or older) diagnosed with prostate cancer scheduled to undergo radical prostatectomy.

2. Standard eligibility criteria for radical prostatectomy.

3. Voluntarily signed the informed consent agreement prior to the performance of any study procedure.

FIG. 1 illustrates an exemplary randomized block design with variable block size for randomly assigning participants to nine treatment groups. After standard quality control analysis of extracted mRNA, 20 samples from patient prostates treated with either finasteride, tamsulosin, or both drugs together for 2, 4, or 6 weeks, were subjected to microarray chip hybridization (in conjunction with the Baylor College of Medicine Microarray Core Facility). The distribution of patient samples is shown in Table 6.

In Table 6, patients were treated with the 5-alpha reductase inhibitor, Finasteride (FIN), or the alpha-1 adrenergic receptor antagonist, Tamsulosin (TAM), or both drugs together (FIN+TAM) for the times indicated prior to prostatectomy. Samples were used in Affymetrix U133 Plus 2.0 microarray analysis as shown.

TABLE 6
Exemplary Finasteride and Tamsulosin Regimen

| | TIME | | |
|---|---|---|---|
| | 2 WEEKS | 4 WEEKS | 6 WEEKS |
| DRUG | Sample ID# | Sample ID# | Sample ID# |
| FIN | 3821 | 3813 | 3773 |
| | 3769 | 1759 | 3770 |
| | | 3775 | |
| | | 2982 | |
| TAM | 3808 | 3774 | 3814 |
| | 3823 | 3812 | |
| | | 3815 | |
| FIN + TAM | 3772 | 3820 | 3811 |
| | 3771 | 3822 | 3809 |

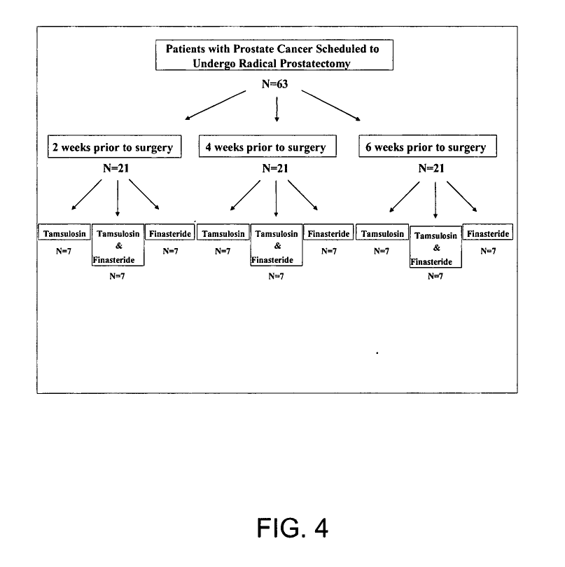

Through this analysis, a number of genes (listed in Table 7) were identified whose expression was modulated by drug treatment. These genes are associated with inflammatory pathways (an exemplary embodiment of which is provided in FIG. 2), Wnt signaling pathway (an exemplary embodiment of which is provided in FIG. 3); cell signaling and cell cycle regulatory pathway (an exemplary embodiment of which is provided in FIG. 4), or extracellular matrix remodeling. Although several of these genes have previously been implicated in prostate cancer progression, they have not been described in connection with progression of BPH or in connection with the exemplary drug treatment regimes described above.

In specific embodiments of the invention, this panel of polynucleotides, or their encoded products, alone and in combination is utilized as diagnostic and/or therapeutic markers of BPH. Also, there are embodiments of the invention concerning these polynucleotides and their participation in one or more respective pathways as therapeutic targets for the treatment of BPH.

In Table 7, there are polynucleotides showing 1.3-77 fold changes in expression level after drug treatment. Down regulation after drug treatment is signified by “-” in front of the fold change. In particular embodiments, the information may be obtained at particular timepoints following the onset of therapy, such as about 1 day, 2 days, 3 days, 4 days, 5 days, 6 days, 1 week, 2 weeks, 3 weeks, 4 weeks, 5 weeks, 6 weeks, 7 weeks, 8 weeks, or combinations thereof, or more or less, for example. In the specific embodiments wherein more than one timepoint is evaluated, an average of the timepoints may be obtained.

In particular embodiments, changes in expression levels of one or more polynucleotides are indicative of therapy with a first drug, a second drug, or a combination thereof. In specific embodiments, the BPH drugs include finasteride, tamsulosin, or both.

TABLE 7
Exemplary Polynucleotides Associated with BPH Drug Therapy

| | Drug | | |
|---|---|---|---|
| | | | Finasteride + |
| Gene | Finasteride | Tamsulosin | Tamsulosin |
| Cbl-b | | 1.30 | 1.37 |
| CKTSF1B1 (Gremlin) | −4.10 | −4.76 | |
| CTNNB1 | | | 1.30 |
| CXCL10 | −2.01 | −4.65 | |
| CXCL11 | −2.62 | −5.81 | |
| CXCL9 | −2.85 | −3.37 | |
| CyclophilinC | −2.52 | −1.87 | −1.64 |
| Dicer 1 | | 1.30 | 1.59 |
| DKK3 | −2.28 | | |
| Frizzled 10 | −2.45 | −1.82 | |
| HLA-DP | −2.29 | −2.33 | |
| HS3ST3B1 | −2.29 | −2.20 | −1.78 |
| KLK4 | −3.47 | | −2.63 |
| MMP12 | | | 3.02 |
| MMP9 | | | 3.66 |
| ORM1 | 2.38 | 76.96 | |
| PRV1 | | | −3.93 |
| SFRP4 | | | 2.51 |
| SPROUTY2 | −1.94 | | |
| VEGF | −2.15 | −2.01 | −3.00 |
| WIF1 | −7.53 | −4.60 | −3.95 |
| ZNF36 | −1.55 | | |

Table 8 provides exemplary sequences and exemplary alternative names for the specific embodiments of the polynucleotides/gene products identified in Table 7.

| | Drug | |
|---|---|---|
| Gene | Sequence Identifier | Exemplary Gene Function |
| Cbl-b | SEQ ID NO: 43 nucleic acid comprises | interacts with SH3 proteins; similar to c-cbl; |
| | Genbank ® Acc. No. U26710; SEQ ID | proto-oncogene product |
| | NO: 44 protein comprises Genbank ® Acc. | |
| | No. AAB09291 | |
| CKTSF1B1 | May also be referred to as | Signaling molecule, such as in |
| (Gremlin) | cysteine knot superfamily 1, BMP | developmental processes; inhibitor of |
| | antagonist 1, GREM1, DRM, PIG2, | bone morphogenetic proteins |
| | DAND2, IHG-2, GREMLIN, CKTSF1B1, | |
| | proliferation-inducing gene 2, or cysteine | |
| | knot superfamily 1, BMP antagonist 1; | |
| | SEQ ID NO: 45 nucleic acid comprises | |
| | Genbank ® Acc. No. NM_013372; SEQ ID | |
| | NO: 46 protein comprises Genbank ® Acc. | |
| | No. NP_037504 | |
| CTNNB1 | Beta catenin (also referred to | Cytoskeletal protein; Signaling molecule |
| | as CTNNB1, catenin (cadherin-associated | |
| | protein), beta 1, 88 kDa or CTNNB; SEQ | |
| | ID NO: 37 nucleic acid comprises | |
| | Genbank ® Acc. No. NM_001904; SEQ ID | |
| | NO: 38 protein comprises Genbank ® Acc. | |
| | No. NP_001895) | |
| CXCL10 | chemokine (C—X—C motif) ligand 10 (also | (IP-10) Inflammation/T cell |
| | referred to as CXCL10, C7, IFI10, INP10, | chemotaxis/IFNγ inducible |
| | IP-10, crg-2, mob-1, SCYB10, or gIP-10; | |
| | SEQ ID NO: 13 nucleic acid comprises | |
| | Genbank ® Acc. No. NM_001565; SEQ ID | |
| | NO: 14 protein comprises Genbank ® Acc. | |
| | No. NP_001556.1) | |
| CXCL11 | chemokine (C—X—C motif) ligand 11 (also | (I-TAC) Inflammation/T cell |
| | referred to as CXCL11, IP9, H174, IP-9, b- | chemotaxis/IFNγ inducible |
| | R1, I-TAC, SCYB11, or SCYB9B; SEQ ID | |
| | NO: 15 nucleic acid comprises Genbank ® | |
| | Acc. No. NM_005409; SEQ ID NO: 16 | |
| | protein comprises Genbank ® Acc. No. | |
| | NP_005400.1) | |
| CXCL9 | chemokine (C—X—C motif) ligand 9 (also | (MIG) Inflammation/T cell |
| | referred to as CXCL9, CMK, MIG, Humig, | chemotaxis/IFNγ inducible |
| | SCYB9, or crg-10; SEQ ID NO: 11 nucleic | |
| | acid comprises Genbank ® Acc. No. | |
| | NM_002416; SEQ ID NO: 12 protein | |
| | comprises Gen Bank Acc. No. | |
| | NP_002407.1) | |
| CyclophilinC | peptidylprolyl isomerase C (cyclophilin C) | IFNγ inducible/CsA binding/ |
| | (also referred to as PPIC, CYPC, or | Ca²⁺ signaling |
| | MGC3673; SEQ ID NO: 9 nucleic acid | |
| | comprises Genbank ® Acc. No. | |
| | NM_000943; SEQ ID NO: 10 protein | |
| | comprises Genbank ® Acc. No. | |
| | NP_000934.1) | |
| Dicer 1 | May also be referred to as | This gene encodes a protein possessing |
| | Dicer, Dcr-1, HERNA, KIAA0928 | an RNA helicase motif containing a DEXH |
| | K12H4.8-LIKE, helicase-moi, or helicase | box in its amino terminus and an RNA |
| | with RNAse motif; SEQ ID NO: 47 nucleic | motif in the carboxy terminus. The encoded |
| | acid comprises Genbank ® Acc. No. | protein functions as a ribonuclease and is |
| | NM_030621; SEQ ID NO: 48 protein | required by the RNA interference and small |
| | comprises Genbank ® Acc. No. NP_085124 | temporal RNA (stRNA) pathways to produce |
| | | the active small RNA component that represses |
| | | gene expression. Two transcript variants |
| | | encoding the same protein have been identified |
| | | for this polynucleotide. |
| DKK3 | dickkopf homolog 3 (also | Wnt inhibitor/tumor invasion/MMP |
| | referred to as DKK3 and REIC; SEQ ID | expression |
| | NO: 5 nucleic acid comprises Genbank ® | |
| | Acc. No. NM_015881; SEQ ID NO: 6 | |
| | protein comprises Genbank ® Acc. No. | |
| | NP_056965.3) | |
| Frizzled 10 | frizzled homolog 10 | Wnt receptor; G protein coupled |
| | (Drosophila) (also referred to as FZD10, | Receptor |
| | FzE7, FZ-10, or hFz10; SEQ ID NO: 1 | |
| | nucleic acid comprises Genbank ® Acc. No. | |
| | NM_007197; SEQ ID NO: 2 protein | |
| | comprises Genbank ® Acc. No. | |
| | NP_009128) | |
| HLA-DP | major histocompatibility complex, class II, | Antigen presentation to T cells |
| | DP alpha 1 (also referred to as HLA-DPA1, | |
| | HLA-D, HLADP, HLASB, HLA-DRA, or | |
| | HLA-DP1A; SEQ ID NO: 7 nucleic acid | |
| | comprises Genbank ® Acc. No. | |
| | NM_033554; SEQ ID NO: 8 protein | |
| | comprises Genbank ® Acc. No. | |
| | NP_291032.2) | |
| HS3ST3B1 | heparan sulfate | Growth factor induction |
| | (glucosamine) 3-O-sulfotransferase 3B1 | Wnt, hedgehog, FGF |
| | (also referred to as HS3ST3B1, 30ST3B1, | |
| | or 3OST3B1; SEQ ID NO: 35 nucleic acid | |
| | comprises Genbank ® Acc. No. | |
| | NM_006041; SEQ ID NO: 36 protein | |
| | comprises Genbank ® Acc. No. | |
| | NP_006032) | |
| KLK4 | kallikrein 4 (prostase, enamel | Serine protease or protease, such as for protein |
| | matrix, prostate) may also be referred to as | metabolism and modification; proteolysis. |
| | ARM1; EMSP; PSTS; EMSP1; KLK-L1; | |
| | PRSS17; prostase; protease, serine, 17; | |
| | kallikrein-like protein 1; androgen- | |
| | regulated message 1; enamel matrix serine | |
| | protease 1; SEQ ID NO: 49 nucleic acid | |
| | comprises Genbank ® Acc. No. | |
| | NM_004917; SEQ ID NO: 50 protein | |
| | comprises Genbank ® Acc. No. NP_004908 | |
| MMP12 | matrix metalloproteinase 12 | Extracellular matrix; metalloprotease; protease |
| | (macrophage elastase); may also be referred | |
| | to as HME, MME, macrophage elastase, or | |
| | macrophage metalloelastase; SEQ ID | |
| | NO: 51 nucleic acid comprises Genbank ® | |
| | Acc. No. NM_002426; SEQ ID NO: 52 | |
| | protein comprises Genbank ® Acc. No. | |
| | NP_002417 | |
| MMP9 | matrix metalloproteinase 9 | Metalloprotease or protease, such as in protein |
| | (gelatinase B, 92 kDa, gelatinase, 92 kDa | metabolism and modification, including proteolysis |
| | type IV collagenase); may also be referred | |
| | to as GELB, CLG4B, gelatinase B, type V | |
| | collagenase, macrophage gelatinase, 92 kD | |
| | type IV collagenase, or matrix | |
| | metalloproteinase 9 (gelatinase B, 92 kD | |
| | gelatinase, 92 kD type IV collagenase); SEQ | |
| | ID NO: 53 nucleic acid comprises | |
| | Genbank ® Acc. No. NM_004994; SEQ ID | |
| | NO: 54 protein comprises Genbank ® Acc. | |
| | No. NP_004985 | |
| ORM1 | orosomucoid 1; may also be | Immunity and defense |
| | referred to as ORM, AGP1, AGP-A, alpha- | |
| | 1-acid glycoprotein 1, or Orosomucoid-1 | |
| | (alpha-1-acid glycoprotein-1); SEQ ID | |
| | NO: 55 nucleic acid comprises Genbank ® | |
| | Acc. No. NM_000607; SEQ ID NO: 56 | |
| | protein comprises Genbank ® Acc. No. | |
| | NP_000598 | |
| PRV1 | polycythemia rubra vera 1; also referred to | Cell surface receptor; Signal transduction; |
| | as NB1, CD177, HNA2A; SEQ ID NO: 57 | Cell surface receptor mediated signal transduction |
| | nucleic acid comprises Genbank ® Acc. No. | |
| | NM_020406; SEQ ID NO: 58 protein | |
| | comprises Genbank ® Acc. No. NP_065139 | |
| SFRP4 | Secreted frizzled related protein 4, also | Cell communication; |
| | referred to as FRP-4 or FRPHE; SEQ ID | Ligand-mediated signaling; Signal |
| | NO: 59 nucleic acid comprises Genbank ® | Transduction |
| | Acc. No. NM_003014; SEQ ID NO: 60 | |
| | protein comprises Genbank ® Acc. No. | |
| | NP_003005 | |
| SPROUTY2 | sprouty homolog 2 | MAPK pathway inhibitor |
| | (Drosophila) (also referred to as SPRY2 | |
| | hSPRY2, or MGC23039; SEQ ID NO: 3 | |
| | nucleic acid comprises Genbank ® Acc. No. | |
| | NM_005842; SEQ ID NO: 4 protein | |
| | comprises Genbank ® Acc. No. | |
| | NP_005833.1) | |
| VEGF | vascular endothelial growth factor, may | Growth factor; signaling molecule; such as for |
| | also be referred to as VEGFA or vascular | cell communication; Receptor protein tyrosine |
| | endothelial growth factor 165b; SEQ ID | kinase signaling pathway; Mesoderm development; |
| | NO: 61 nucleic acid comprises Genbank ® | Ligand-mediated signaling; Intracellular |
| | Acc. No. NM_003376; SEQ ID NO: 62 | signaling cascade; Angiogenesis; Oncogenesis; |
| | protein comprises Genbank ® Acc. No. | Signal transduction; MAPKKK cascade; |
| | NP_003367 | Developmental processes; Other oncogenesis; |
| | | Cell surface receptor mediated signal transduction |
| WIF1 | WNT inhibitory factor 1 (WIF1), also | Signaling molecule; Cell communication; |
| | referred to as WIF-1; SEQ ID NO: 63 | Ligand-mediated signaling; |
| | nucleic acid comprises Genbank ® Acc. No. | Signal transduction |
| | NM_007191; SEQ ID NO: 64 protein | |
| | comprises Genbank ® Acc. No. NP_009122 | |
| ZNF36 | zinc finger with KRAB and SCAN domains | Transcription factor; Zinc finger transcription factor; |
| | 1 (ZKSCAN1); may also be referred to as | or KRAB box transcription factor, such as for mRNA |
| | KOX18, ZNF36, PHZ-37, ZNF139, or | transcription regulation; Nucleoside, nucleotide |
| | 9130423L19Rik; SEQ ID NO: 65 nucleic | and nucleic acid metabolism; or mRNA transcription |
| | acid comprises Genbank ® Acc. No. | |
| | NM_003439; SEQ ID NO: 66 protein | |
| | comprises Genbank ® Acc. No. NP_003430 | |
| | Dermatopontin (nucleic acid | Regulator of TGF-beta signaling |
| | in Genbank ® Acc. No. NM_001937 (SEQ | |
| | ID NO: 39); protein in Genbank ® Acc. No. | |
| | NP_001928; SEQ ID NO: 40) | |

### Example 4

### Exemplary Identification of Diagnostic Polynucleotides/Gene Products

In particular embodiments of the invention, there is a method of identifying one or more polynucleotides and/or their encoded gene products as indicative of response to a therapy. In an exemplary embodiment, tissue from a prostate of a male over the age of about 40 is obtained, such as with biopsy. The individual is suspected to be at risk for BPH, has BPH, is to be subjected to BPH therapy, or is receiving BPH therapy. In alternative embodiments, cells are obtained from serum, blood, urine, needle aspirate, prostate fluid, and so forth.

RNA is obtained from one or more of the cells and subjected to microchip analysis, wherein the level of the RNAs is analyzed. A variety of genes are identified as having upregulation or downregulation compared to a control. For example, a first polynucleotide may be upregulated compared to a control, and a second polynucleotide may be downregulated compared to a control. The controls may be provided in a kit, for example, and they may be obtained from an individual known not to have BPH. The corresponding expression levels of the exemplary first and second polynucleotides are indicative of the individual being or at risk for becoming resistant to a BPH therapy, such as finasteride or transulosin, or both. An alternative BPH therapy is, therefore, employed, such as surgery or a minimally invasive procedure, such as microwave treatment, radiation treatment, an alternative drug, or a combination thereof, for example.

### Example 5

### Specific Embodiments of the Invention

In one specific embodiment of the invention, it is characterized whether the following markers are detectable in the urine and serum of patients with BPH and are predictors of clinically important BPH disease-related endpoints: Chemokines MIG (CXCL9), IP-10 (CXCL10), and I-TAC (CXCL11), adhesion molecule Contactin-1 (CNTN1) or Wnt pathway inhibitor Dickkopf-3 (DKK3), for example. A bead-based protein array assay is developed to measure simultaneously all five of these markers in a single patient sample. To further characterize the clinical significance of these exemplary predictors of the presence, severity or progression of BPH, they are each quantitated in urine and/or serum samples, for example, obtained from multiple suitable patients. These patients will encompass a wide range of prostatic diseases including benign prostatic hyperplasia (BPH) and prostate cancer, for example, and may be treated with a range of medical therapy regimes and other therapies, for example. In additional specific embodiments, these exemplary markers are quantitated in a control population of patients without prostate disease. Prediction models that either include or that do not include these markers are constructed to determine the incremental improvement in disease prediction that these markers allow, in specific embodiments of the invention.

In an additional specific embodiment of the invention, it is characterized whether the following markers are clinically significant predictors of the progression of BPH: Chemokines MIG (CXCL9), IP-10 (CXCL10), and I-TAC (CXCL11), adhesion molecule Contactin-1 (CNTN1) or Wnt pathway inhibitor Dickkopf-3 (DKK3). In particular aspects, all five of these exemplary markers are quantified using a bead protein array, for example, that requires only a 100 μl aliquot in the cohort of baseline serum samples from patients. Baseline nomogram models that predict both symptom progression endpoints and AUR/surgery endpoints have been constructed and are available freely for use on the web, such as at the Oncovance website. Furthermore, the inventors previously measured BPSA, proPSA and free PSA in baseline serum samples and demonstrated that BPSA was an independent predictor of AUR/surgery, in particular aspects of the invention. In this specific embodiment, additional nomogram models are generated that include these five exemplary markers to determine if the accuracy of prediction of clinically important endpoints is improved, as determined by the concordance index.

Associated with specific embodiments of the present invention are the following exemplary polynucleotides, some of which are described in other tables above.

Table 9 shows exemplary polynucleotides that are differentially expressed between big (≧70 g) and small (≦30 g) prostates with a 2 fold or greater change in expression level. GM is the gemetric mean of the normalized signal intensity and represents the relative levels of individual gene expression in the prostate tissue as measured on the Affymetrix U133 Plus 2.0 microarrays. Accession numbers are from Genbank®. Array data was analyzed using dChip.

TABLE 9

| | | | | |
|---|---|---|---|---|
| GM | | Fold | | |
| Small | GM Big | change | Accession# | Description |

Genes Up-Regulated in Big Prostate

| | | | | |
|---|---|---|---|---|
| 164.58 | 638.22 | 3.88 | AI091445 | contactin 1 |
| 56.45 | 218.68 | 3.87 | AW072790 | contactin 1 |
| 37.15 | 124.18 | 3.34 | AF030514 | chemokine (C—X—C motif) |
| | | | | ligand 11 |
| 97.79 | 314.57 | 3.22 | AL050154 | Homo sapiens mRNA; cDNA |
| | | | | DKFZp586L0120 |
| 90.08 | 264.53 | 2.94 | NM_001565 | chemokine (C—X—C motif) |
| | | | | ligand 10 |
| 51.88 | 151.52 | 2.92 | AA780067 | heparan |
| | | | | sulfate(glucosamine) 3-O- |
| | | | | sulfotransferase 3B1 |
| 106.39 | 289.75 | 2.72 | AI376433 | KIAA1912 protein |
| 88.98 | 223.82 | 2.52 | NM_002416 | chemokine (C—X—C motif) |
| | | | | ligand 9 |
| 99.68 | 249.07 | 2.50 | AI963203 | solute carrier family 7 |
| | | | | (cationic amino acid |
| | | | | transporter; y+ system); |
| | | | | member 3 |
| 114.86 | 280.59 | 2.44 | AI806909 | Homo sapiens transcribed |
| | | | | sequences |
| 122.80 | 298.92 | 2.43 | AL157471 | Fraser syndrome 1 |
| 150.46 | 364.75 | 2.42 | AL390170 | Homo sapiens LOC347294 |
| | | | | (LOC347294); mRNA |
| 54.80 | 127.99 | 2.34 | AI743621 | collagen; type I; alpha 1 |
| 68.19 | 157.43 | 2.31 | NM_013381 | thyrotropin-releasing |
| | | | | hormone degrading |
| | | | | ectoenzyme |
| 106.14 | 244.38 | 2.30 | AA700440 | Homo sapiens LOC343202 |
| | | | | (LOC343202); mRNA |
| 164.16 | 373.20 | 2.27 | NM_003278 | tetranectin (plasminogen |
| | | | | binding protein) |
| 247.95 | 562.84 | 2.27 | NM_000943 | peptidylprolyl isomerase C |
| | | | | (cyclophilin C) |
| 104.13 | 233.90 | 2.25 | AI799018 | Homo sapiens; Similar to |
| | | | | nuclear localization signals |
| | | | | binding protein 1 |
| 264.59 | 592.25 | 2.24 | U33428 | potassium voltage-gated |
| | | | | channel; shaker-related |
| | | | | subfamily; beta member 1 |
| 241.27 | 532.86 | 2.21 | BC007230 | coagulation factor C |
| | | | | homolog; cochlin (Limulus |
| | | | | polyphemus) |
| 110.05 | 242.37 | 2.20 | NM_001548 | interferon-induced protein |
| | | | | with tetratricopeptide |
| | | | | repeats 1 |
| 95.93 | 210.46 | 2.19 | NM_000114 | endothelin 3 |
| 26.60 | 57.60 | 2.17 | AW004016 | beta-galactoside alpha-2;6 |
| | | | | sialyltransferase II |
| 60.41 | 128.91 | 2.13 | BG222258 | Homo sapiens cDNA |
| | | | | FLJ30333 fis; clone |
| | | | | BRACE2007262. |
| 56.35 | 118.27 | 2.10 | AL553774 | KIAA1462 protein |
| 215.01 | 449.47 | 2.09 | AL045306 | nudix (nucleoside |
| | | | | diphosphate linked moiety |
| | | | | X)-type motif 10 |
| 25.75 | 53.22 | 2.07 | R97781 | Homo sapiens transcribed |
| | | | | sequences |
| 39.63 | 81.11 | 2.05 | NM_152903 | hypothetical protein |
| | | | | DKFZp547E1912 |
| 339.35 | 689.33 | 2.03 | NM_013253 | dickkopf homolog 3 |
| | | | | (Xenopus laevis) |
| 174.24 | 352.28 | 2.02 | BF724270 | transcriptional regulating |
| | | | | protein 132 |
| 7.72 | 15.57 | 2.02 | Z38765 | Homo sapiens transcribed |
| | | | | sequences |
| 499.37 | 1003.14 | 2.01 | BE962749 | peptidylprolyl isomerase C |
| | | | | (cyclophilin C) |

Genes Down-Regulated in Big Prostate

| | | | | |
|---|---|---|---|---|
| 337.45 | 167.13 | −2.02 | AL589866 | kraken-like |
| 271.94 | 130.59 | −2.08 | AI742383 | Homo sapiens transcribed |
| | | | | sequences |
| 548.74 | 253.04 | −2.17 | NM-001584 | chromosome 11 open |
| | | | | reading frame 8 |
| 451.28 | 205.66 | −2.19 | AI813772 | Bardet-Biedl syndrome 4 |
| 228.41 | 102.81 | −2.22 | AI435590 | Homo sapiens transcribed |
| | | | | sequences |
| 457.52 | 190.65 | −2.40 | AB066566 | activating transcription |
| | | | factor 3 | |
| 696.44 | 232.20 | −3.00 | U80034 | mitochondrial intermediate |
| | | | | peptidase |
| 1020.67 | 337.85 | −3.02 | NM_030751 | transcription factor 8 |
| | | | | (represses interleukin 2 |
| | | | | expression) |
| 492.16 | 160.60 | −3.06 | AL022718 | Homo sapiens cDNA clone |
| | | | | IMAGE: 4811759; partial cds |
| 512.06 | 167.07 | −3.06 | AA833830 | Homo sapiens cDNA |
| | | | | FLJ35632 fis; clone |
| | | | | SPLEN2011678. |
| 1096.60 | 254.09 | −4.32 | AF254357 | folate hydrolase (prostate- |
| | | | | specific membrane antigen) 1 |
| 857.34 | 120.77 | −7.10 | NM_005059 | relaxin 2 (H2) |
| 2970.56 | 382.89 | −7.76 | AF003934 | prostate differentiation factor |
| 942.14 | 118.68 | −7.94 | BG032839 | Homo sapiens; clone |
| | | | | IMAGE: 4429946; mRNA |

Three exemplary embodiments of the invention are addressed as follows: 1) that zone-specific transcriptional profiling of the prostate identifies gene sets that correlate with clinical and pathologic parameters of BPH; 2) that medical therapy for BPH using selective α1-receptor blockade, 5α-reductase type II inhibition, or both, results in changes in prostate TZ gene expression indicative of response to therapy; and 3) that gene expression profiling is indicative of protein expression changes associated with pathologic BPH and in specific embodiments leads to the identification of putative biomarkers for disease presence, progression, and/or response to treatment.

In pursuit of the first specific embodiment (above), the inventors isolated TZ BPH nodular tissue from prostates of less than 30 g (small) and from prostates of greater than 70 g (big) in patients undergoing radical prostatectomy. RNA from a total of 26 individual patients (16 small prostates and 10 big prostates) was purified and RNA quality assessed. RNA from 9 of these samples were judged to be of insufficient quality for microarray analysis. In total, gene expression profiles were analyzed on 17 samples (9 small and 8 big) using Affymetrix U133 Plus 2.0 whole genome microarrays. Of these 17 samples on which arrays were run, 2 were ultimately excluded from the final analysis due to excessive 3′ degradation or inadequate signal strength as determined by the internal quality control parameters within the arrays. The remaining 15 (9 small and 6 big) array results were normalized and analyzed using dChip (Li and Wong, 2001). 409 genes showed statistically significant differences (by t test; p≦0.01) in expression levels between the small and big prostates of which 46 showed differential expression of 2 fold or greater (Table 9).

To address the second specific embodiment, patients awaiting radical prostatectomy were given a standard therapeutic course of tamsulosin, finasteride or both tamsulosin and finasteride for 2, 4, or 6 weeks prior to surgery. TZ BPH nodular tissue was isolated post surgery. Total RNA was purified from 24 samples from individual patients and analyzed using Affymetrix U133 Plus 2.0 microarrays. Of the 24 arrays run on drug treated patient prostates, 7 were ultimately excluded from the final analysis due poor signal quality within the arrays. The categorical distribution of the remaining arrays is shown in Table 10, which includes TZ BPH prostate tissue samples from a total of 17 individual patients treated with either tamsulosin, finasteride or both for the times indicated that were used in the microarray analysis.

TABLE 10
Distribution of Patient Samples of TZ BPH Prostate Tissue

| | 2 weeks | 4 weeks | 6 weeks |
|---|---|---|---|
| Tamsulosin | 2 | 2 | 2 |
| Finasteride | 2 | 2 | 1 |
| Tamsulosin/Finasteride | 2 | 2 | 2 |

Expression levels from drug treated prostate tissue were analyzed with dChip. Comparison of changes in gene expression between drugs and between time of therapy was performed by ANOVA. This initial analysis yielded 3568 genes with significant a difference (p≦0.01) in at least 1 comparison. Of these genes several hundred showed at least one change of greater than 2 fold. Genes regulated by drug treatment were grouped by pathway or common function and those showing multiple changes in expression are shown in Table 11.

TABLE 11
A selection of the genes with greater than 2 fold differential
expression that are regulated by finasteride and tamsulosin therapy.
Differential gene expression is shown for treatment with drug between
2 and 6 weeks. Blue are proinflammatory genes,
yellow are matrix or prostate proteases, and green are genes
of the Wnt signaling pathway. CXCL9 shown in red was regulated
by all treatments.

| | | | Finasteride + |
|---|---|---|---|
| Gene Drug | Finasteride | Tamsulosin | Tamsulosin |
| CXCL9 | −3.61 | −2.86 | −4.04 |
| VEGF | −2.15 | −2.01 | −3.00 |
| CKTSF1B1 (Gremlin) | −4.10 | −4.76 | |
| HLA-DP | −2.29 | −2.33 | |
| HS3ST3B1 | −2.29 | −2.20 | |
| ORM1 | 2.38 | 76.96 | |
| PRV1 | | | −3.93 |
| KLK4 | −3.47 | | |
| MMP12 | | | 3.02 |
| MMP9 | | | 3.66 |
| DKK3 | | −2.85 | |
| Frizzled 10 | −2.45 | | |
| CTNNB1 | 2.26 | | 2.57 |
| SFRP4 | | | 2.51 |
| WIF1 | −7.53 | −4.60 | −3.95 |

A comparison of genes differentially expressed in the small versus big pro states with those pro states treated with finasteride or tamsulosin or both (Tables 9 and 11), shows that several genes occur in both experiments. Genes appearing in both analyses and those with the highest fold changes in expression levels in either experiment were considered most likely to be of interest to understanding the biology of BPH and to marker development.

To further refine the list of genes profiled in the microarray studies, the differential expression of 30 of these genes were tested by real-time quantitative RT-PCR (Q-RT-PCR). Since Q-RT-PCR is based on the detection of small amplimers (80-150 base pairs) it. is less affected by RNA degradation than microarrays. This allowed the inventors to used a larger sample set of prostate RNA than were included in the microarray analyses. The inventors tested 16 small and 10 big prostate samples (including the 15 samples from the array analysis) and 24 samples from drug treated patients (10 treated with finasteride, 7 treated with tamsulosin, and 7 treated with both drugs). Individual comparisons were made between small and big prostates and between no drug (all 18 small and big samples) and drug treatments for each therapy drug condition without consideration of the treatment duration. Genes with significant expression differences (p≦0.05) as determined by t test are shown in Table 12.

Several groups of genes validated with significant differences by Q-RT-PCR between small and big prostate and/or those regulated by at least one drug treatment. The three pro-inflammatory chemokines CXCL9 (MIG), CXCL10 (IP-10), and CXCL11 (I-TAC) are best known for production by monocytes as chemotactic agents for T cells (Kim and Broxmeyer, 1999). Interestingly, these three chemokines all act through the same G protein coupled receptor CXCR3 and are induced by interferon-γ (IFN γ). Though these chemokines have never, to the knowledge of the inventors, been described in the prostate, they have been observed in other stromal and epithelial tissues (Goldberg-Bittman et al., 2005; Kitaya et al., 2004). The fact that all three are soluble compounds, present in serum, upregulated in BPH nodules, and are correspondingly decreased by treatment with finasteride or tamsulosin, makes them potentially useful markers if they are expressed in the prostate. In addition, normal prostate stroma and basal epithelia have been shown to express IFNγ and its receptors, which is significantly up regulated by these cells in BPH and cancer (Royuela et al., 2000).

Contactin 1 is a GPI-linked cell surface adhesion molecule that is well described in neural tissue where it is thought to direct the branching and proliferation of myelinated axons. Contactin 1, like other GPI anchored molecules, is susceptible to cleavage by phosphotidylinositol-phospholipase C12 or other enzymes making it detectable potentially as a serum marker. Contactin 1 has been described as an adhesion ligand for neural cell adhesion molecule (NCAM, CD56) (Theodosis et al., 2000) and for tenascins C and R (Rigato et al., 2002; Zacharias et al., 2002) both of which are expressed in prostate (Li et al., 2003; Tuxhom et al., 2002). Contactin 1 has also recently been shown to be a ligand for the notch receptor, where it causes notch ICD nuclear translocation at levels similar to the archetypal notch ligands of the delta/serrate/jagged families (Hu et al., 2003). Notch expression has been shown in prostate tissue (Shou et al., 2001; Wang et al., 2004), but to the knowledge of the inventors, contactin 1 has not been described in the prostate. Additionally, Heyl, a transcriptional repressor that mediates Notch signaling, is also capable of repressing androgen receptor (AR) signaling (Belandia et al., 2005). Signaling through AR supports the survival and proliferation of prostate cells in BPH22 and Hey1 has been shown to colocalizes with AR in BPH epithelia but not in prostate cancers (Belandia et al., 2005). In specific embodiments of the invention, these data indicate that Notch signaling has a functional role in the propagation of BPH that may be mediated potentially through contactin 1 interaction.

The Wnt signaling pathway has been implicated in a variety of cancers including those of the prostate (Cronauer et al., 2005; Yardy and Brewster, 2005). The Q-RT-PCR validated array data on BPH tissue shows significant differential regulation of several Wnt pathway members by one or more drug treatments. These include the Wnt inhibitors, DKK3, sFRP4 and WIF1, Wnt receptor Frizzled 10 and Wnt signaling intermediate β-Catenin. Taken together these results indicate that Wnt signaling plays a role in BPH pathology, in particular aspects of the invention. Of the 5 exemplary Wnt pathway genes that show differential expression, only the soluble Wnt inhibitor DKK3 shows a significantly increased expression level between small and big prostates as well as a compensatory significant decrease in expression by drug treatment. DKK3 has been implicated as a tumor suppressor gene that is down regulated in prostate and other cancers (Hsieh et al., 2004). Although, to the knowledge of the inventors, no formal description of DKK3 expression in BPH has been published; a search of the online array data base, ONCOMINE (see the Oncomine website on the internet), lists 2 microarray studies where DKK3 expression is compared between BPH and prostate cancer (Dhanasekaran et al., 2001; Luo et al., 2001). Examination of the microarray data in both these studies shows that DKK3 is significantly down-regulated in prostate cancer compared to BPH tissue and that DKK3 is up-regulated (though not significant; p=0.058) in BPH compared to normal prostate (Dhanasekaran et al., 2001). The array data indicates that the secreted factor DKK3 is a useful marker for the differentiation of BPH and prostate cancer, in specific embodiments.

### Example 6

### Expression of Potential BPH Markers in Whole Prostate Tissue

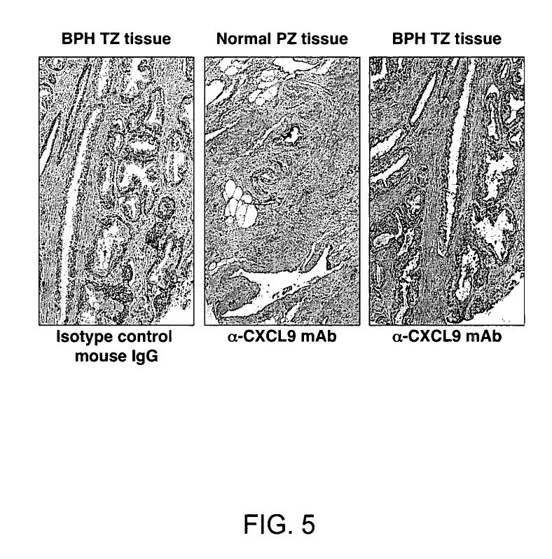

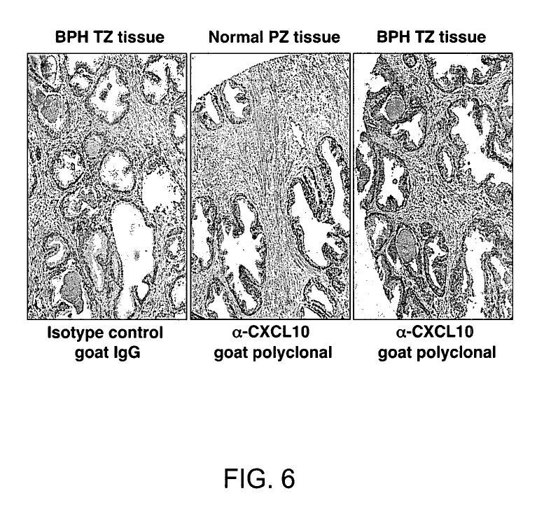

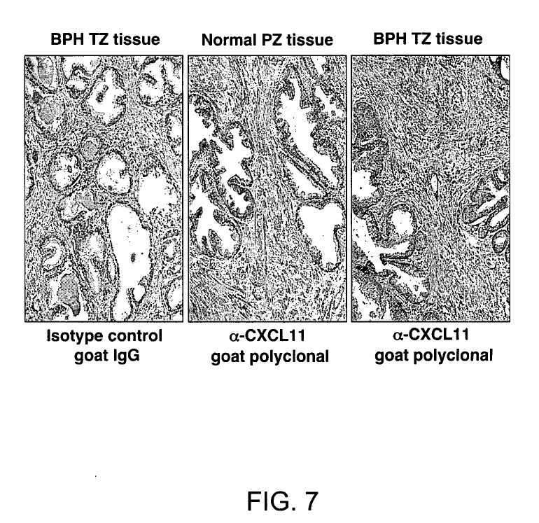

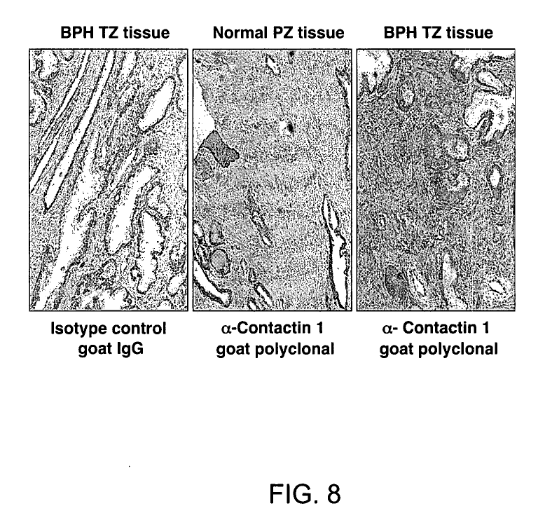

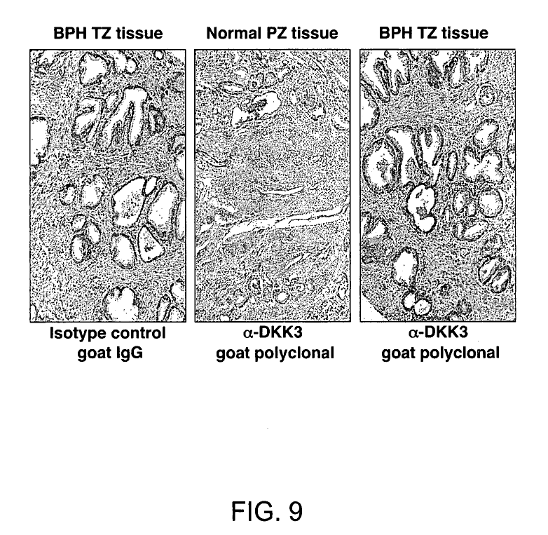

To determine the specific cellular and regional expression of the exemplary BPH markers, transition zone BPH nodular tissue was isolated as 2 mm punches from paraffin embedded prostates and was stained using standard immunohistochemical techniques with antibodies specific for CXCL9, CXCL10, CXCL11, Contactin 1 or DKK3. Expression of CXCL9 was detected in the stroma of both TZ and PZ tissue, but was prominent in the glandular epithelia of BPH TZ (FIG. 5). CXCL10 expression was detected at low levels in stroma and epithelium of TZ and PZ prostate with no apparent difference in the level of expression between BPH and adjacent control tissue (FIG. 6). CXCL11 was expressed in both the stroma and epithelia of normal and BPH TZ (FIG. 7). Although we have not quantified the level of CXCL11 staining in these experiments this chemokine appears to be up-regulated in the stroma of BPH tissues. Contactin 1 expression was detectable in the epithelia of normal PZ,but was low to negative in stromal cells from the control tissue (FIG. 8). In contrast Contactin 1 expression was strikingly absent from epithelial cells of TZ and highly expressed in the stromal compartment of this BPH tissue. DKK3 expression was detectable in the stroma and epithelium of the TZ but was only barely detectable in the PZ (FIG. 9). Taken together these data demonstrate that these 5 potential markers for BPH are all expressed at the protein level in prostate tissue.

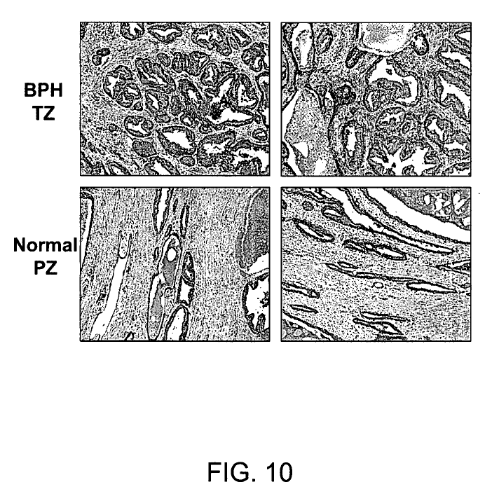

That the chemokines CXCL9, 10 and 11 all signal through the same receptor (CXCR3) and were all detected in the arrays indicates that they may function directly within the prostate in addition to potentially generating inflammatory signals to infiltrating lymphocytes. Function in situ within the prostate presupposes that CXCR3 is also expressed in by prostate cells. The inventors stained PZ and TZ tissue sections with a mAb for CXCR3 (FIG. 10). CXCR3 was expressed in both PZ and TZ epithelium but appears to be expressed only in the stroma of the TZ. These data indicate that this family of interferon-inducible chemokines has the potential to function directly on prostate tissue.

### Example 7

### Expression of Potential BPH Markerts in Prostate Cell Lines

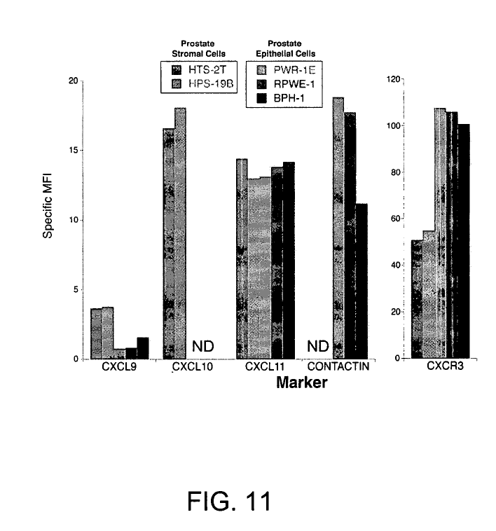

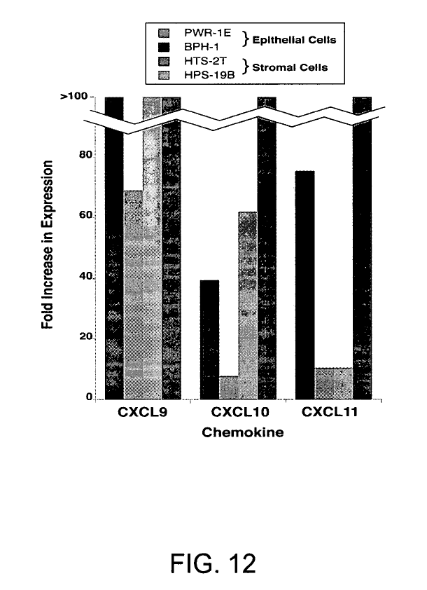

To confirm the expression of CXCL9, 10 and 11, contactin 1 and CXCR3 in prostate tissue, the present inventors examined several non-cancer prostate cell lines for these markers. The prostate epithelial cells RWPE-1 and PWR-1E are androgen responsive nomal or hyperplastic cells transformed with HPV1828, 29. The epithelial cell line BPH 130 is originated from BPH tissue transformed with SV40 T antigen. The cell lines HTS-2T and HPS-19B are primary prostate stromal cell lines from TZ and PZ, respectively (Singh et al., 2004). Cells were cultured in their prescribed media and were fixed, permeabilized and stained by standard methods and analyzed by single color flow cytometry. The chemokines CXCL9, 10 and 11 were present in all cells tested as was contactin 1 (FIG. 11). DKK3 was not tested for lack of a suitable antibody for flow cytometric analysis. CXCR3 was highly expressed in all the cells. Induction of these potential BPH markers was examined in cells cultured in the the presence of the synthetic testosterone analog R1881 or inflammatory agents LPS or IL-8. No changes in marker expression were observed following exposure to androgen or the inflammatory mediators. To determine if prostate cell expression of CXCL chemokines was responsive to interferon stimulation, stromal and epithelial cell lines were cultured in the presence of IFNγ. Chemokine induction was measured by Q-RT-PCR and is shown as fold induction over basal expression in untreated cells (FIG. 12). CXCL9 expression, which showed the lowest basal expression by intracellular staining, was induced at the greatest levels. In most of the cell lines tested CXCL9 expression increased excess of 100 fold over basal levels. Induction of CXCL10 and CXCL11 by IFNγ. ranged between 8 and greater than 100 fold over basal levels depending on the individual cell line. Taken together these data confirm that CXCL 9, 10, and 11, and contactin are expressed and measurable in prostate tissue.

### Example 8

### Measurement of CXCL Chemokine Markers in Serum and Urine

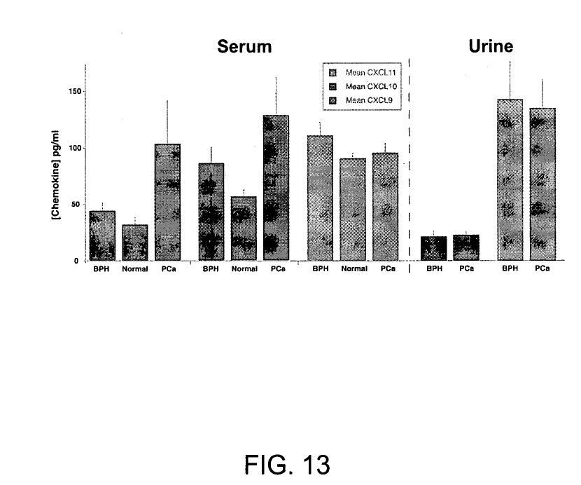

For the genes validated in the microarray studies to be of use as potential biomarkers in the detection and/or staging of BPH, they are detectable in biological fluids, in specific embodiments. A recent study by Hu et al. has shown that CXCL9, 10 , and 11 were detectable at significant levels in the urine of kidney transplant patients where they were diagnostic for organ failure (Hu et al., 2004). To determine if one could detect these markers in the urine of patients with prostate disease, the present inventors collected urine samples from 20 patients who were being treated for either BPH or prostate cancer. Expression of these CXCL chemokines was measure in the urine samples by commercially available ELISA (R&D systems). Additionally, a small cohort of banked serum taken from clinic patients treated for BPH or prosdtate cancer was also tested (FIG. 13). CXCL 9, 10 and 11 were all detectable in the serum from patients with BPH, prostate cancer, and normal controls (n=13 for each group). No significant differences in chemokine levels were seen in this small sampling. CXCL 10 and CXCL11 were detectable in the urine from patients with BPH or prostate cancer (n=20 for each group), but CXCL9 was detectable in only a very few samples. Taken together these data indicate that these chemokines are expressed at sufficient levels in the serum and urine of patients with prostate disease to be pursued as potential biomarkers.

### Example 9

### Exemplary Experimental Procedures for Examples 6-8

The following exemplary experimental procedures may be employed in the invention.

Development of a 5-Marker Bead-Based Protein Array

Commercial ELISAs for the detection of CXCL9, 10 and 11 are available from several manufacturers, but to the knowledge of the inventors there are no commercially available assays for the detection of either contactin 1 or DKK3. In specific embodiments of the invention, all five markers are evaulated from serum and urine in a large number of samples, and in further specific embodiments, a multimarker bead array assay is employed. The principle of bead arrays is similar to that of a sandwich ELISA, employing a specific capture and detection antibody pair for each marker. Capture antibodies are bound to small beads of varying size or color, with a unique bead type used for each marker. A mixture of beads containing the specific capture antibodies for each marker are mixed with the biological sample, washed, and then combined with a mixture of the fluorescent labeled detection antibodies. Once the sandwich complex is completed, the beads are analyzed, such as by flow cytometry, for example. Marker concentration is determined by fluorescent intensity and different markers are distinguished by the specifics of the bead properties (size or color, for example). Bead array assays aside from multiplexing have a significant advantage over ELISAs in that there are fewer sources of error in the process. Where ELISAs are sensitive to minute volume fluctuations, particularly the final substrate volume used to read the assay, bead arrays are independent of final volume. Bead arrays collect individual data for each bead (as many as 5000 data points per marker), which is then averaged, allowing fluctuations in the normality of the bead signal distribution to flag potential errors in the measurement of each sample.

Protein bead arrays are available from several manufacturers that sell unconjugated beads with unique characteristics. The inventors have recently identified sources of commercially available antibodies suitable for the detection of contactin 1 and DKK3 in fluid samples and also sources of contactin 1 and DKK3 recombinant proteins for use as protein standards. Matched antibody pairs for the detection of CXCL 9, 10 and 11 are commonly available, as are the standards. Once the assay is in place the inventors are able to measure all markers simultaneously in duplicate in a 96 well format, for example. The total volume of sample required will be about 100 μl, for example, in certain aspects of the invention.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents and Patent Applications

U.S. Pat. No. 5,399,363

U.S. Pat. No. 5,466,468

U.S. Pat. No. 5,543,158

U.S. Pat. No. 5,580,579

U.S. Pat. No. 5,629,001

U.S. Pat. No. 5,641,515

U.S. Pat. No. 5,725,871

U.S. Pat. No. 5,756,353

U.S. Pat. No. 5,780,045

U.S. Pat. No. 5,792,451

U.S. Pat. No. 5,804,212,

U.S. Pat. No. 5,912,135

U.S. Pat. No. 6,048,888

U.S. Pat. No. 6,384,035

U.S. Pat. No. 6,410,554

U.S. Pat. No. 6,613,308

U.S. Pat. No. 6,733,779

### PUBLICATIONS

Belandia, B., S. M. Powell, J. M. Garcia-Pedrero, M. M. Walker, C. L. Bevan, and M. G. Parker. 2005. Heyl, a mediator of notch signaling, is an androgen receptor corepressor. Mol Cell Biol 25:1425-36.

Bello, D., M. M. Webber, H. K. Kleinman, D. D. Wartinger, and J. S. Rhim. 1997. Androgen responsive adult human prostatic epithelial cell lines immortalized by human papillomavirus 18. Carcinogenesis 18:1215-23.

Berry, S. J., D. S. Coffey, P. C. Walsh, and L. L. Ewing. 1984. The development of human benign prostatic hyperplasia with age. J Urol 132:474-9.

Coffey, D. S., S. J. Berry, and L. L. Ewing, An overview of current concepts in the study of benign prostatic hyperplasia, in Benign Prostatic Hyperplasia, C. H. Rodgers, Editor. 1987, NIH: Bethesda. p. 1-13.

Cronauer, M. V., W. A. Schulz, R. Ackermann, and M. Burchardt. 2005. Effects of WNT/beta-catenin pathway activation on signaling through T-cell factor and androgen receptor in prostate cancer cell lines. Int J Oncol 26:1033-40.

Dhanasekaran, S. M., T. R. Barrette, D. Ghosh, R. Shah, S. Varambally, K. Kurachi, K. J. Pienta, M. A. Rubin, and A. M. Chinnaiyan. 2001. Delineation of prognostic biomarkers in prostate cancer. Nature 412:822-6.

Franks, L. M. 1954. Benign nodular hyperplasia of the prostate: A review. Ann Royal Coll Surg Engl 14:92-106.

Goldberg-Bittman, L., O. Sagi-Assif, T. Meshel, I. Nevo, O. Levy-Nissenbaum, I. Yron, I. P. Witz, and A. Ben-Baruch. 2005. Cellular characteristics of neuroblastoma cells: regulation by the ELR-CXC chemokine CXCL10 and expression of a CXCR3-like receptor. Cytokine 29:105-17.

Guess, H. A. 1992. Benign prostatic hyperplasia: antecedents and natural history. Epidemiol Rev 14:131-53.

Hayward, S. W., R. Dahiya, G. R. Cunha, J. Bartek, N. Deshpande, and P. Narayan. 1995. Establishment and characterization of an immortalized but non-transformed human prostate epithelial cell line: BPH-1. In vitro Cell Dev Biol Anim 31:14-24.

Hsieh, S. Y., P. S. Hsieh, C. T. Chiu, and W. Y. Chen. 2004. Dickkopf-3/REIC functions as a suppressor gene of tumor growth. Oncogene 23:9183-9.

Hu, H., B. D. Aizenstein, A. Puchalski, J. A. Burmania, M. M. Hamawy, and S. J. Knechtle. 2004. Elevation of CXCR3-binding chemokines in urine indicates acute renal-allograft dysfunction. Am J Transplant 4:432-7.

Hu, Q. D., et al. 2003. F3/contactin acts as a functional ligand for Notch during oligodendrocyte maturation. Cell 115:163-75.

Hwang S J, Park H, Park K. Gastric retentive drug-delivery systems. Crit Rev Ther Drug Carrier Syst. 1998;15(3):243-84.

Kim, C. H. and H. E. Broxmeyer. 1999. Chemokines: signal lamps for trafficking of T and B cells for development and effector function. J Leukoc Biol 65:6-15.

Kitaya, K., T. Nakayama, N. Daikoku, S. Fushiki, and H. Honjo. 2004. Spatial and temporal expression of ligands for CXCR3 and CXCR4 in human endometrium. J Clin Endocrinol Metab 89:2470-6.

Koch, T., T. Brugger, A. Bach, G. Gennarini, and J. Trotter. 1997. Expression of the immunoglobulin superfamily cell adhesion molecule F3 by oligodendrocyte-lineage cells. Glia 19:199-212.

Kyprianou, N., J. Chon, and C. M. Benning. 2000. Effects of alpha(1)-adrenoceptor (alpha(1)-AR) antagonists on cell proliferation and apoptosis in the prostate: therapeutic implications in prostatic disease. Prostate Suppl 9:42-6.

Li, C. and W. H. Wong. 2001. Model-based analysis of oligonucleotide arrays: expression index computation and outlier detection. Proc Natl Acad Sci U S A 98:31-6.

Li, R., T. Wheeler, H. Dai, and G. Ayala. 2003. Neural cell adhesion molecule is upregulated in nerves with prostate cancer invasion. Hum Pathol 34:457-61.

Luo, J., D. J. Duggan, Y. Chen, J. Sauvageot, C. M. Ewing, M. L. Bittner, J. M. Trent, and W. B. Isaacs. 2001. Human prostate cancer and benign prostatic hyperplasia: molecular dissection by gene expression profiling. Cancer Res 61:4683-8.

Mathiowitz E, Jacob J S, Jong Y S, Carino G P, Chickering D E, Chaturvedi P, Santos C A, Vijayaraghavan K, Montgomery S, Bassett M, Morrell C. Biologically erodable microspheres as potential oral drug delivery systems. Nature. 1997; 386(6623):410-4.

Nantermet, P. V., et al. 2004. Identification of genetic pathways activated by the androgen receptor during the induction of proliferation in the ventral prostate gland. J Biol Chem 279:1310-22.

Rigato, F., J. Garwood, V. Calco, N. Heck, C. Faivre-Sarrailh, and A. Faissner. 2002. Tenascin-C promotes neurite outgrowth of embryonic hippocampal neurons through the alternatively spliced fibronectin type III BD domains via activation of the cell adhesion molecule F3/contactin. J Neurosci 22:6596-609.

Royuela, M., M. P. de Miguel, A. Ruiz, B. Fraile, M. I. Arenas, E. Romo, and R. Paniagua. 2000. Interferon-gamma and its functional receptors overexpression in benign prostatic hyperplasia and prostatic carcinoma: parallelism with c-myc and p53 expression. Eur Cytokine Netw 11:119-27.

Shou, J., S. Ross, H. Koeppen, F. J. de Sauvage, and W. Q. Gao. 2001. Dynamics of notch expression during murine prostate development and tumorigenesis. Cancer Res 61:7291-7.

Singh, H., T. D. Dang, G. E. Ayala, and D. R. Rowley. 2004. Transforming growth factor-beta1 induced myofibroblasts regulate LNCaP cell death. J Urol 172:2421-5.

Takenaga M, Serizawa Y, Azechi Y, Ochiai A, Kosaka Y, Igarashi R, Mizushima Y. Microparticle resins as a potential nasal drug delivery system for insulin. J Control Release. 1998 Mar 2;52(1-2):81-7.

Theodosis, D. T., K. Pierre, and D. A. Poulain. 2000. Differential expression of two adhesion molecules of the immunoglobulin superfamily, F3 and polysialylated NCAM, in hypothalamic magnocellular neurones capable of plasticity. Exp Physiol 85 Spec No:187S-196S.

Tuxhorn, J. A., G. E. Ayala, M. J. Smith, V. C. Smith, T. D. Dang, and D. R. Rowley. 2002. Reactive stroma in human prostate cancer: induction of myofibroblast phenotype and extracellular matrix remodeling. Clin Cancer Res 8:2912-23.

Wang, X. D., J. Shou, P. Wong, D. M. French, and W. Q. Gao. 2004. Notch1-expressing cells are indispensable for prostatic branching morphogenesis during development and re-growth following castration and androgen replacement. J Biol Chem 279:24733-44.

Webber, M. M., D. Bello, H. K. Kleinman, D. D. Wartinger, D. E. Williams, and J. S. Rhim. 1996. Prostate specific antigen and androgen receptor induction and characterization of an immortalized adult human prostatic epithelial cell line. Carcinogenesis 17:1641-6.

Yardy, G. W. and S. F. Brewster. 2005. Wnt signalling and prostate cancer. Prostate Cancer Prostatic Dis.

Zacharias, U., R. Leuschner, U. Norenberg, and F. G. Rathjen. 2002. Tenascin-R induces actin-rich microprocesses and branches along neurite shafts. Mol Cell Neurosci 21:626-33.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of identifying a risk of developing benign prostatic hyperplasia (BPH) and/or detecting the presence of BPH in an individual, comprising the step of identifying a change in a level of one or more polynucleotides or an encoded product thereof, wherein the encoded product is a member of an inflammatory pathway, a Wnt signaling pathway, a cell signaling pathway, a cell cycle pathway, or a combination thereof.

2. The method of claim 1, wherein the polynucleotide or encoded product is identified in Table 3, Table 4, Table 5, Table 9, or a combination thereof.

3. The method of claim 1, wherein the identifying step is further defined as comprising:
obtaining a sample from the individual; and
detecting a change in level of one or more nucleic acid sequences or the encoded product thereof in Table 3, Table 4, Table 5, Table 9, or a combination thereof.

4. The method of claim 3, wherein the sample comprises biopsy, needle aspirate, prostate fluid, serum, blood, or urine.

5. The method of claim 1, wherein the individual has a prostate size larger than about 30 grams.

6. The method of claim 3, wherein the detecting step comprises microarray analysis, polymerase chain reaction, immunoblot analysis, immunoassay, proteomic assay, or a combination thereof.

7. The method of claim 1, further comprising evaluating an additional risk factor.

8. The method of claim 7, wherein the risk factor is age, race, total PSA level, free PSA level, % Free PSA, BPSA level, -2proPSA level, maximum urine flow rate, AUA SI, BPH impact index, PVR, ultrasound total prostate volume, ultrasound TZ volume, or a combination thereof.

9. The method of claim 1, wherein when the individual is identified as being at risk for developing BPH or is identified as having BPH, the individual is administered a therapy.

10. The method of claim 9, wherein the therapy comprises surgery.

11. The method of claim 9, wherein the therapy is a minimally invasive therapy.

12. The method of claim 11, wherein the minimally invasive therapy comprises microwave treatment, radiofrequency treatment, delivery of therapeutic composition, or a combination thereof.

13. The method of claim 1, wherein the encoded product is further defined as a member of an inflammatory pathway, a Wnt pathway, a cell signaling pathway, a cell cycle pathway, an extracellular matrix remodeling pathway, or a combination thereof.

14. The method of claim 13, wherein the one or more polynucleotides or encoded products thereof are identified in Table 7.

15. The method of claim 13, wherein the individual is further defined as an individual who is to receive a BPH therapy or who is receiving a BPH therapy and wherein the level of one or more expressed polynucleotides from an individual is compared to a control, wherein a difference between the level of at least one expressed polynucleotide predicts the response to BPH therapy in the individual.

16. The method of claim 15, wherein the BPH therapy is further defined as a 5-alpha reductase inhibitor, an alpha-1 adrenergic receptor antagonist, or a mixture thereof.

17. The method of claim 15, wherein the BPH therapy is finasteride, tamsulosin, or a mixture thereof.

18. The method of claim 15, wherein when the method predicts the individual as being refractory to the BPH therapy, the individual is subjected to an alternative BPH therapy.

19. A method of predicting or evaluating the response of an individual to a benign prostatic hyperplasia (BPH) therapy, comprising the steps of identifying a change in a level of one or more polynucleotides or an encoded product thereof, wherein the encoded product is a member of an inflammatory pathway, a Wnt pathway, a cell signaling pathway, a cell cycle pathway, an extracellular matrix remodeling pathway, or a combination thereof.

20. A method of determining performance for one or more drug therapies for BPH in an individual, comprising the step of identifying the expression level of one or more polynucleotides in Table 7 in the individual.

21. The method of claim 20, further defined as comparing the level of one or more nucleic acid sequences in Table 7 in one or more prostate cells of the individual with the level of one or more nucleic acid sequences in Table 7 from one or more cells that are known to be sensitive to the BPH drug therapy.

22. As a composition of matter, isolated expressed polynucleotides the levels of which are indicative of a risk for developing benign prostatic hyperplasia (BPH), and/or the presence of BPH.

23. The composition of claim 22, wherein one or more of the expressed polynucleotides are identified in Table 3, Table 4, Table 5, or Table 9.

24. The composition of claim 22, wherein the expressed polynucleotides are comprised on a substrate.

25. The composition of claim 24, wherein the substrate comprises a microarray chip.

26. The composition of claim 22, further comprising BPH risk factor-evaluating information, BPH risk factor-evaluating reagent, or a combination thereof.

27. The composition of claim 26, wherein the BPH risk factor-evaluating information comprises statistical information for prostate related to age, race, total PSA level, free PSA level, % Free PSA, BPSA level, -2proPSA level, maximum urine flow rate, AUA SI, BPH impact index, PVR, ultrasound total prostate volume, ultrasound TZ volume, or a combination thereof.

28. The composition of claim 26, wherein the BPH risk factor-evaluating reagent comprises a PSA level measuring reagent, a maximum urine flow rate measuring reagent, or both.

29. As a composition of matter, isolated expressed polynucleotides the levels of which are indicative of susceptibility to BPH, and/or the presence of BPH, wherein one or more of the expressed polynucleotides are listed in Table 7.

30. A pharmaceutical composition, wherein the composition is capable of treating benign prostatic hyperplasia and comprises an agent that targets a member of an inflammatory pathway, a Wnt signaling pathway, a cell signaling pathway, a cell cycle pathway, or a combination thereof.

31. A method of treating an individual for BPH, comprising the step of providing to the individual an agent that targets an inflammatory pathway, a Wnt pathway, a cell signaling pathway, a cell cycle pathway, or a combination thereof.

32. A kit housed in a suitable container, comprising a composition that identifies expression level of one or more polynucleotides of Table 3, Table 4, Table 5, Table 7, or Table 9.

33. A kit housed in a suitable container, comprising an agent that targets a polynucleotide identified in Table 3, Table 4, Table 5, or Table 7, or the encoded product thereof.


SEQUENCE LISTING (66 sequences)

SEQ ID NO: 1 - DNA, 3260 bp, Human
tcgaaacagctgccggctggtcccggccgaggccggcgcagggagggaggagccgcccgggctgtgggggcgccgcgagctgggccggcctcggtgtgcccgcgccgccagcccgctccagacgcgccacctgggcgctccaagaagaggccgaagtttgccgcggccgtgagttggagctcgcgccgggccgctgcgccgggagctccgggggcttccctcgcttcccggtattgtttgcaaactttgctgctctccgccgcggcccccaactcggcggacgccgggcgcggagagccgagccgggggcgctgtgcgcagcgctcgggccaggccgggcgggcatgggcgggggcccgagcaggggtggagagccggggccagcagcagcccgtgcccgggagcggcggcgctgaggggcgcggagctccccgcgaggacacgtccaacgccagcatgcagcgcccgggcccccgcctgtggctggtcctgcaggtgatgggctcgtgcgccgccatcagctccatggacatggagcgcccgggcgacggcaaatgccagcccatcgagatcccgatgtgcaaggacatcggctacaacatgactcgtatgcccaacctgatgggccacgagaaccagcgcgaggcagccatccagttgcacgagttcgcgccgctggtggagtacggctgccacggccacctccgcttcttcctgtgctcgctgtacgcgccgatgtgcaccgagcaggtctctacccccatccccgcctgccgggtcatgtgcgagcaggcccggctcaagtgctccccgattatggagcagttcaacttcaagtggcccgactccctggactgccggaaactccccaacaagaacgaccccaactacctgtgcatggaggcgcccaacaacggctcggacgagcccacccggggctcgggcctgttcccgccgctgttccggccgcagcggccccacagcgcgcaggagcacccgctgaaggacgggggccccgggcgcggcggctgcgacaacccgggcaagttccaccacgtggagaagagcgcgtcgtgcgcgccgctctgcacgcccggcgtggacgtgtactggagccgcgaggacaagcgcttcgcagtggtctggctggccatctgggcggtgctgtgcttcttctccagcgccttcaccgtgctcaccttcctcatcgacccggcccgcttccgctaccccgagcgccccatcatcttcctctccatgtgctactgcgtctactccgtgggctacctcatccgcctcttcgccggcgccgagagcatcgcctgcgaccgggacagcggccagctctatgtcatccaggagggactggagagcaccggctgcacgctggtcttcctggtcctctactacttcggcatggccagctcgctgtggtgggtggtcctcacgctcacctggttcctggccgccggcaagaagtggggccacgaggccatcgaagccaacagcagctacttccacctggcagcctgggccatcccggcggtgaagaccatcctgatcctggtcatgcgcagggtggcgggggacgagctcaccggggtctgctacgtgggcagcatggacgtcaacgcgctcaccggcttcgtgctcattcccctggcctgctacctggtcatcggcacgtccttcatcctctcgggcttcgtggccctgttccacatccggagggtgatgaagacgggcggcgagaacacggacaagctggagaagctcatggtgcgtatcgggctcttctctgtgctgtacaccgtgccggccacctgtgtgatcgcctgctacttttacgaacgcctcaacatggattactggaagatcctggcggcgcagcacaagtgcaaaatgaacaaccagactaaaacgctggactgcctgatggccgcctccatccccgccgtggagatcttcatggtgaagatctttatgctgctggtggtggggatcaccagcgggatgtggatttggacctccaagactctgcagtcctggcagcaggtgtgcagccgtaggttaaagaagaagagccggagaaaaccggccagcgtgatcaccagcggtgggatttacaaaaaagcccagcatccccagaaaactcaccacgggaaatatgagatccctgcccagtcgcccacctgcgtgtgaacagggctggagggaagggcacaggggcgcccggagctaagatgtggtgcttttcttggttgtgtttttctttcttcttcttctttttttttttttataaaagcaaaagagaaatacataaaaaagtgtttaccctgaaattcaggatgctgtgatacactgaaaggaaaaatgtacttaaagggttttgttttgttttggttttccagcgaagggaagctcctccagtgaagtagcctcttgtgtaactaatttgtggtaaagtagttgattcagccctcagaagaaaacttttgtttagagccctccctaaatatacatctgtgtatttgagttggctttgctacccatttacaaataagaggacagataactgctttgcaaattcaagagcctcccctgggttaacaaatgagccatccccagggcccacccccaggaaggccacagtgctgggcggcatccctgcagaggaaagacaggacccggggcccgcctcacaccccagtggatttggagttgcttaaaatagactccggccttcaccaatagtctctctgcaagacagaaacctccatcaaacctcacatttgtgaactcaaacgatgtgcaatacatttttttctctttccttgaaaataaaaagagaaacaagtattttgctatatataaagacaacaaaagaaatctcctaacaaaagaactaagaggcccagccctcagaaacccttcagtgctacattttgtggctttttaatggaaaccaagccaatgttatagacgtttggactgatttgtggaaaggaggggggaagagggagaaggatcattcaaaagttacccaaagggcttattgactctttctattgttaaacaaatgatttccacaaacagatcaggaagcactaggttggcagagacactttgtctagtgtattctcttcacagtgccaggaaagagtggtttctgcgtgtgtatatttgtaatatatgatatttttcatgctccactattttattaaaaataaaatatgttctttagtttgctgctaaaaaaa

SEQ ID NO: 2 - protein, 581 aa, Human
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFAVVWLAIWAVLCFFSSAFTVLTFLIDPARFRYPERPIIFLSMCYCVYSVGYLIRLFAGAESIACDRDSGQLYVIQEGLESTGCTLVFLVLYYFGMASSLWWVVLTLTWFLAAGKKWGHEAIEANSSYFHLAAWAIPAVKTILILVMRRVAGDELTGVCYVGSMDVNALTGFVLIPLACYLVIGTSFILSGFVALFHIRRVMKTGGENTDKLEKLMVRIGLFSVLYTVPATCVIACYFYERLNMDYWKILAAQHKCKMNNQTKTLDCLMAASIPAVEIFMVKIFMLLVVGITSGMWIWTSKTLQSWQQVCSRRLKKKSRRKPASVITSGGIYKKAQHPQKTHHGKYEIPAQSPTCV

SEQ ID NO: 3 - DNA, 2126 bp, Human
gtaaggccgttttcttttcccattcgctcatctgccaggaaaagggacttgccgttggcgcttcggcctcttgttcattgagaaaaaagaggaaatactccgcgtgcgcttgtagaaggggagtcgtctccagctccgaaccccggagtgttcatcagcggggaatctggctccgaattctctttttttctcccgccgattgctcggaagttggtctaaagcagaggttggaaagaaaggaaaaaagtttgcatcgagactggatttatttgcacatcgcagaaagaagagaatccaagggagaggggttggtgcaaagccgcgatcacggagttcagatgtgttctaagcctgctggagtgaccacacttccaagacctgatggaggccagagctcagagtggcaacgggtcgcagcccttgctgcagacgccccgtgacggtggcagacagcgtggggagcccgaccccagagacgccctcacccagcaggtacatgtcttgtctctggatcagatcagagccatccgaaacaccaatgagtacacagaggggcctactgtcgtcccaagacctgggctcaagcctgctcctcgcccctccactcagcacaaacacgagagactccacggtctgcctgagcaccgccagcctcctaggctccagcactcgcaggtccattcttctgcacgagcccctctgtccagatccataagcacggtcagctcagggtcgcggagcagtacgaggacaagtaccagcagcagctcctctgaacagagactgctaggatcatccttctcctccgggcctgttgctgatggcataatccgggtgcaacccaaatctgagctcaagccaggtgagcttaagccactgagcaaggaagatttgggcctgcacgcctacaggtgtgaggactgtggcaagtgcaaatgtaaggagtgcacctacccaaggcctctgccatcagactggatctgcgacaagcagtgcctttgctcggcccagaacgtgattgactatgggacttgtgtatgctgtgtgaaaggtctcttctatcactgttctaatgatgatgaggacaactgtgctgacaacccatgttcttgcagccagtctcactgttgtacacgatggtcagccatgggtgtcatgtccctctttttgccttgtttatggtgttaccttccagccaagggttgccttaaattgtgccaggggtgttatgaccgggttaacaggcctggttgccgctgtaaaaactcaaacacagtttgctgcaaagttcccactgtcccccctaggaactttgaaaaaccaacatagcatcattaatcaggaatattacagtaatgaggattttttctttctttttttaatacacatatgcaaccaactaaacagttataatcttggcactgttaatagaaaggtgggatagtctttgctgtttgcggtgaaatgctttttgtccatgtgccgttttaactgatatgcttgttagaactcagctaatggagctcaaagtatgagatacagaacttggtgacccatgtattgcataagctaaagcaacacagacactcctaggcaaagtttttgtttgtgaatagtacttgcaaaacttgtaaattagcagatgacttttttccattgttttctccagagagaatgtgctatatttttgtatatacaataatatttgcaactgtgaaaaacaagttgtgccatactacatggcacagacacaaaatattatactaatatgttgtacattcggaagaatgtgaatcaatcagtatgtttttagattgtattttgccttacagaaagcctttattgtaagactctgatttccctttggacttcatgtatattgtacagttacagtaaaattcaacctttattttctaattttttcaacatattgtttagtgtaaagaatatttatttgaagttttattattttataaaaaagaatatttattttaagaggcatcttacaaattttgccccttttatgaggatgtgatagttgctgcaaatgaggggttacagatgcatatgtccaatataaaatagaaaatatattaacgtttgaaattaaaaaaaaaaaaaaaaaa

SEQ ID NO: 4 - protein, 315 aa, Human
MEARAQSGNGSQPLLQTPRDGGRQRGEPDPRDALTQQVHVLSLDQIRAIRNTNEYTEGPTVVPRPGLKPAPRPSTQHKHERLHGLPEHRQPPRLQHSQVHSSARAPLSRSISTVSSGSRSSTRTSTSSSSSEQRLLGSSFSSGPVADGIIRVQPKSELKPGELKPLSKEDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 5 - DNA, 2686 bp, Human
cgacgccaaggggagggggctgacctgtgcttggtccaccccaggtaggggctgagagaggcttgaggtggaagtgggggtcgggcactctgacctggtcgaggaggggctagggtttgaaccggggacagagtctaggtgagctggggcttgggagctattagcgtagaggatccgggttcggttgctctggcgagggctccagcatcacaggcggcggctgcgggcgcagagcggagatgcagcggcttggggccaccctgctgtgcctgctgctggcggcggcggtccccacggcccccgcgcccgctccgacggcgacctcggctccagtcaagcccggcccggctctcagctacccgcaggaggaggccaccctcaatgagatgttccgcgaggttgaggaactgatggaggacacgcagcacaaattgcgcagcgcggtggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgagggagcctgcggctgccgccgctgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttcccctctggcttgacagcatgaggtgttgtgcatttgttcagctcccccaggctgttctccaggcttcacagtctggtgcttgggagagtcaggcagggttaaactgcaggagcagtttgccacccctgtccagattattggctgctttgcctctaccagttggcagacagccgtttgttctacatggctttgataattgtttgaggggaggagatggaaacaatgtggagtctccctctgattggttttggggaaatgtggagaagagtgccctgctttgcaaacatcaacctggcaaaaatgcaacaaatgaattttccacgcagttctttccatgggcataggtaagctgtgccttcagctgttgcagatgaaatgttctgttcaccctgcattacatgtgtttattcatccagcagtgttgctcagctcctacctctgtgccagggcagcattttcatatccaagatcaattccctctctcagcacagcctggggagggggtcattgttctcctcgtccatcagggatctcagaggctcagagactgcaagctgcttgcccaagtcacacagctagtgaagaccagagcagtttcatctggttgtgactctaagctcagtgctctctccactaccccacaccagccttggtgccaccaaaagtgctccccaaaaggaaggagaatgggatttttcttttgaggcatgcacatctggaattaaggtcaaactaattctcacatccctctaaaagtaaactactgttaggaacagcagtgttctcacagtgtggggcagccgtccttctaatgaagacaatgatattgacactgtccctctttggcagttgcattagtaactttgaaaggtatatgactgagcgtagcatacaggttaacctgcagaaacagtacttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaattatgtataaacataagttgcatttagaaatcaagcataaatcacttcaactgctaaaaaaaaaaaaaaaaaaaaaaaaaaaaaaaaaaaa

SEQ ID NO: 6 - protein, 350 aa, Human
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 7 - DNA, 1157 bp, Human
gctcacagtcatcaattatagaccccacaacatgcgccctgaagacagaatgttccatatcagagctgtgatcttgagagccctctccttggctttcctgctgagtctccgaggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcatcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcactgtcaccttcccgagaataccctaagaccaataaatacttcagtatttc

SEQ ID NO: 8 - protein, 260 aa, Human
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 9 - DNA, 1241 bp, Human
agcgcgcaggcgcagccggccggcagtcccgtcagctgtcccagagcctgtgtcgcgcccgtgccggtagcgcccgtgccggtagcgccgctgccaccgctcaccatgggcccgggtcctcggctgctgctacctctcgtgctttgcgtggggctcggcgcacttgtgttttcttcgggggccgagggcttccgcaagcgaggcccctcggtgacggccaaggtcttctttgatgtgaggattggagacaaagatgttggcagaattgtgattggcctctttggaaaagttgtgcccaagacagtggaaaattttgttgctctagcaacaggagagaaaggatatggatataaaggaagcaagtttcatcgtgtcatcaaggatttcatgattcaaggaggtgacatcaccactggagatggcactgggggtgtgagcatctatggtgagacatttccagatgagaacttcaagctgaagcactatggcattgggtgggtcagcatggccaacgctgggcctgacaccaatggctctcagttctttatcaccttgaccaagcccacctggttggacggcaaacatgtggtgtttggaaaagtcattgatgggatgacagtggtgcactccatagagctccaagcaactgatgggcatgaccgtccactcaccaactgctcgatcatcaacagtggcaagatagacgtgaaaacgccttttgtggttgagatcgctgattggtgacacaactggcagaaaacaaggatatgctttggcaggggtgtgtgtgtgtgtgtgtgtgtgtgtgtgttgtgttgtctttcaattatttgcttttttttttttactttctttttgtattctatcccagatcacaggaaagttataaaaatcaaaccgtcaccctttagtttgcttgaactttagtaaaccacctgcttagggactttgaacttaaatatatccccttcctcaagtggtgctattttaaaactaaaaaaaactttgaattggctatttttttaatgcaatattttttttctgaattcattatgatccccatattgggtaatgctgaacatttatctgaaacagatgaggatattattattttgtatccaaacagaaattcagataaagggaaatttgactagtgtaatctgagatatgtcatagggatttctttctgacaaaagggtgctttgctgttctttatattaaatacttttagatcaaaaaaa

SEQ ID NO: 10 - protein, 212 aa, Human
MGPGPRLLLPLVLCVGLGALVFSSGAEGFRKRGPSVTAKVFFDVRIGDKDVGRIVIGLFGKVVPKTVENFVALATGEKGYGYKGSKFHRVIKDFMIQGGDITTGDGTGGVSIYGETFPDENFKLKHYGIGWVSMANAGPDTNGSQFFITLTKPTWLDGKHVVFGKVIDGMTVVHSIELQATDGHDRPLTNCSIINSGKIDVKTPFVVEIADW

SEQ ID NO: 11 - DNA, 2545 bp, Human
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 12 - protein, 125 aa, Human
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 13 - DNA, 1172 bp, Human
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact

SEQ ID NO: 14 - protein, 98 aa, Human
MNQTAILICCLIFLTLSGIQGVPLSRTVRCTCISISNQPVNPRSLEKLEIIPASQFCPRVEIIATMKKKGEKRCLNPESKAIKNLLKAVSKEMSKRSP

SEQ ID NO: 15 - DNA, 1493 bp, Human
ttcctttcatgttcagcatttctactccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacacttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 16 - protein, 94 aa, Human
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 17 - DNA, 2530 bp, Human
aaatccttcttccaatgttcctcccctctctgtatgaaccctgtgttggggggcagaagatggaagcccttggcaagctcgatcgaaccaagctactaaattgctgagctcgttttaactgaagtgtgagaaggaggtttaaggcaagtagacaacatcctgttgttggggtgcttctctcttttttgcacatctggctgaactgggagtcaggtggttgacttgtgcctggctgcagtagcagcggcatctcccttgcacagttctcctcctcggcctgcccaagagtccaccaggccatggacgcagtggctgtgtatcatggcaaaatcagcagggaaaccggcgagaagctcctgcttgccactgggctggatggcagctatttgctgagggacagcgagagcgtgccaggcgtgtactgcctatgtgtgctgtatcacggttacatttatacataccgagtgtcccagacagaaacaggttcttggagtgctgagacagcacctggggtacataaaagatatttccggaaaataaaaaatctcatttcagcatttcagaagccagatcaaggcattgtaatacctctgcagtatccagttgagaagaagtcctcagctagaagtacacaaggtactacagggataagagaagatcctgatgtctgcctgaaagccccatgaagaaaaataaaacaccttgtactttattttctataatttaaatatatgctaagtcttatatattgtagataatacagttcggtgagctacaaatgcatttctaaagccattgtagtcctgtaatggaagcatctagcatgtcgtcaaagctgaaatggacttttgtacatagtgaggagctttgaaacgaggattgggaaaaagtaattccgtaggttattttcagttattatatttacaaatgggaaacaaaaggataatgaatactttataaaggattaatgtcaattcttgccaaatataaataaaaataatcctcagtttttgtgaaaagctccatttttagtgaaatattattttatagctactaattttaaaatgtcttgcttgattgtatggtgggaagttggctggtgtcccttgtctttgccaagttctccactagctatggtgtcataggctcttttgggatttttgaagctgtatactgtgtgctaaaacaagcactaaacaaagagtgaaggatttatgtttaattctgaaagcaaccttcttgcctagtgttctgatattggacagtaaaatccacagaccaacctggagttgaaaatcttataatttaaaatatgctctaaacatgtttatcgtatttgatgctacaggatttgaaattgtattacaaatccaatgaaatgagtttttcttttcatttacctctgccccagttgtttctactacatggaagacctcattttgaagggaaatttcagcagctgcagctcatgagtaactgatttgtaacaagcctccttttaaagtaaccctacaaaaccactggaaagtttatggttgtattattttttaaaaaaattccaagtgattgaaacctacacgagatacagaattttatgcggcattttcttctcacatttatatttttgtgattttgtgattgattatatgtcactttgctacagggctcacagaattcattcactcaacaaacataatagggcgctgagggcatagaagtaaaaacacctggtccctgctctcagttcactgtcttgttggacgagaaaagaaacaataacgataaaagacagtgaaagaaaataacgataaaagacagtgaaagaaaataacaataaaagacaaggaaaaaataacaatgaaagttgataagtacatgataagcgaggttccccgtgtgtaggtagatctggtctttagaggcagatagataggtcagtgcaaatactctggtccatgggccatatgaaaaggctaagcttcactgtaaaataataactgggaattctggattgtgtatgggtgttggtgaacttggttttaattagtgaactgctgagagacagagctattctccatgtactggcaagacctgatttctgagcatttaatatggatgccgtgggagtacaaaagtggagtgtggcctgagtaatgcattatgggtggtttaccatttcttgaggtaaaagcatcacatgaacttgtaaaggaatttaaaaatcctactttcataataagttgcataggtttaataatttttaattatatggcttgagtttaaattgtaataggcgtaactaattttaactctataatgtgttcattctggaataatcctaaacatatgaattatgtttgcatgttcacttccaagagcctttttttgaaaaaaagctttttttgaatcatcaagtctttcacatttaaataaagtgtttgaaagctttatttaaaaaaaaaaaaaaaaaaaaaaaaaaaaaaaaaaaaaaaaaaaaaaaaaaaaaaaaaaaaaaaaaaaaagaaaaaaa

SEQ ID NO: 18 - protein, 128 aa, Human
MDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 19 - DNA, 4723 bp, Human
atgcggcgcggccccggaggcagcagcagcggcggcggcagccggagcagtaggcacccgagcagcgccagcggccgagcgggcggcttcctggcctgggcgctccggtggcggcggaggtgcgcgcggagccatggttatcatgtcggagttcagcgcggaccccgcgggccagggtcagggccagcagaagcccctccgggtgggtttttacgacatcgagcggaccctgggcaaaggcaacttcgcggtggtgaagctggcgcggcatcgagtcaccaaaacgcaggttgcaataaaaataattgataaaacacgattagattcaagcaatttggagaaaatctatcgtgaggttcagctgatgaagcttctgaaccatccacacatcataaagctttaccaggttatggaaacaaaggacatgctttacatcgtcactgaatttgctaaaaatggagaaatgtttgattatttgacttccaacgggcacctgagtgagaacgaggcgcggaagaagttctggcaaatcctgtcggccgtggagtactgtcacgaccatcacatcgtccaccgggacctcaagaccgagaacctcctgctggatggcaacatggacatcaagctggcagattttggatttgggaatttctacaagtcaggagagcctctgtccacgtggtgtgggagccccccgtatgccgccccggaagtctttgaggggaaggagtatgaaggcccccagctggacatctggagcctgggcgtggtgctgtacgtcctggtctgcggttctctccccttcgatgggcctaacctgccgacgctgagacagcgggtgctggagggccgcttccgcatccccttcttcatgtctcaagactgtgagagcctgatccgccgcatgctggtggtggaccccgccaggcgcatcaccatcgcccagatccggcagcaccggtggatgcgggctgagccctgcttgccgggacccgcctgccccgccttctccgcacacagctacacctccaacctgggcgactacgatgagcaggcgctgggtatcatgcagaccctgggcgtggaccggcagaggacggtggagtcactgcaaaacagcagctataaccactttgctgccatttattacctcctccttgagcggctcaaggagtatcggaatgcccagtgcgcccgccccgggcctgccaggcagccgcggcctcggagctcggacctcagtggtttggaggtgcctcaggaaggtctttccaccgaccctttccgacctgccttgctgtgcccgcagccgcagaccttggtgcagtccgtcctccaggccgagatggactgtgagctccagagctcgctgcagtggcccttgttcttcccggtggatgccagctgcagcggagtgttccggccccggcccgtgtccccaagcagcctgctggacacagccatcagtgaggaggccaggcaggggccgggcctagaggaggagcaggacacgcaggagtccctgcccagcagcacgggccggaggcacaccctggccgaggtctccacccgcctctccccactcaccgcgccatgtatagtcgtctccccctccaccacggcaagtcctgcagagggaaccagctctgacagttgtctgaccttctctgcgagcaaaagccccgcggggctcagtggcaccccggccactcaggggctgctgggcgcctgctccccggtcaggctggcctcgcccttcctggggtcgcagtccgccaccccagtgctgcaggctcaggggggcttgggaggagctgttctgctccctgtcagcttccaggagggacggcgggcgtcggacacctcactgactcaagggctgaaggcctttcggcagcagctgaggaagaccacgcggaccaaagggtttctgggactgaacaaaatcaaggggctggctcgccaggtgtgccaggtccctgccagccgggccagcaggggcggcctgagccccttccacgcccctgcacagagcccaggcctgcacggcggcgcagccggcagccgggagggctggagcctgctggaggaggtgctagagcagcagaggctgctccagttacagcaccacccggccgctgcacccggctgctcccaggccccccagccggcccctgccccgtttgtgatcgccccctgtgatggccctggggctgccccgctccccagcaccctcctcacgtcggggctcccgctgctgccgcccccactcctgcagaccggcgcgtccccggtggcctcagcggcgcagctcctggacacacacctgcacattggcaccggccccaccgccctccccgctgtgcccccaccacgcctggccaggctggccccaggttgtgagcccctggggctgctgcagggggactgtgagatggaggacctgatgccctgctccctaggcacgtttgtcctggtgcagtgagggcagccctgcatcctggcacggacactgactcttacagcaataacttcagaggaggtgaagacatctggcctcaaagccaagaactttctagaagcgaaataagcaatacgttaggtgttttggctttttagtttatttttgttttatttttttcttgcactgagtgacctcaactttgagtagggactggaaactttaggaagaaagataattgaggggcgtgtctgggggcgggggcaggaggggagcggggtggagggaacacgtgcagtgccgtggtgtggggatctcggcccctctctctgggttcgtcgtggttgagatgattacctcggacgtctacggaaacgagcgggcgcattgttgtccgcttgtgtgtgtgtgtgtgtgtgtgtgtgtgcgcgtgcattgattactatccatttctttagtcaacgctctccacttcctgatttctgctttaaggaaaactgtgaactttctgcttcatgtatcagttttaaagcagcccaggcaaagatcatctacagattctaggaattctctcccctgaaatcaaaacctggaagacttttttttcttattttagttgagaagtttcataaactgctcaaggattagttttccaggactctgcggaggaacggcaggaagaacctcagagagggcagaggtgacttcaaagtgctggggactccgtcctgagggtcacttggccctgagcccctgcgtgcccttgcggaagcccagaagcttcttcctgctgcacctcccgtttccgctgctgctgacgtttatgcatttcatgatggggtccaacaagaacacctgacttgggtgaagttgtgcaatattggaggctgactgtagggctgggcagctgggagacaggctcatggctcatggctcatggctcagggcggtgcctgccctgggccgggacccccctccccaccccccacctaggctttttgggttttgttcaaggaaggtaaagtgagaggtttaggtcagtgtttttaagtttttgttttttttttaaagcaaatcctgtatatgtatctacatgggagacaggtagacactacttatttgttacattttgtactatacgtttgtgttccaggtttcagcttccctcgctcctgttgttaagaagcgtccctgtcagcacaggtgtgcattgaggaaggggccccagggccttcgctccctcagcactggggtggaggcggcaggaaggggcggcccttacctggcaggtctgggcgcacctttagcaggtggactccgtggggctccaccagccagaagcctctggaaggcaacgaaggcaatgctgctccctgagtccagtccccgcccccaaacccagcccaggtgccttcagctacttcggcttcttaaaccctgcagtgttaaacagaggcattgagaaaggggaaaggcgggtatttttaaaagccaaagattgacccagttacttgagggtagggaggcgggcccagtgcaggaggctgcatccctggcctgctggtgcccaccgggggctgtgcctgtgccgggccgcagggaagctggctgcccccattcctgctgctgctgctgctgctgctctgtggctgtttcaaagactgggcgaaaggctgtccggagggcagaccaggtgccttgccgcagagaaaacaccaaagtctcctgttcgctcataaagaagtttttgggatgggagagaatccagaccatcttggggcagccaggcccttgccttcatttttacagaggtagcacaactgattccaacacaaaaccccttcccctttttaaaatgatttctgttctaatgccatagatcaaaggcctcagaaaccattgtgtgtttcctctttgaagcaatgacaagcactttactttcacggtggtttttgttttttcttattgctgtggaacctcttttggaggacgttaaaggcgtgttttacttgtttttttaagagtgtgtgatgtgtgttttgtagatttcttgacagtgctgtaatacagacggcaatgcaatagcctatttaaagacactacgtgatctgattgagatgtacatagtttttttttttaccataactgaattattttatctcttatgttaacatgagaaatgtatgccaaatgattagttgatgtatgttttttaatttaatatttaaataaaatatttggaaggaaaaaaaaaaaaaaaaa

SEQ ID NO: 20 - protein, 783 aa, Human
MVIMSEFSADPAGQGQGQQKPLRVGFYDIERTLGKGNFAVVKLARHRVTKTQVAIKIIDKTRLDSSNLEKIYREVQLMKLLNHPHIIKLYQVMETKDMLYIVTEFAKNGEMFDYLTSNGHLSENEARKKFWQILSAVEYCHDHHIVHRDLKTENLLLDGNMDIKLADFGFGNFYKSGEPLSTWCGSPPYAAPEVFEGKEYEGPQLDIWSLGVVLYVLVCGSLPFDGPNLPTLRQRVLEGRFRIPFFMSQDCESLIRRMLVVDPARRITIAQIRQHRWMRAEPCLPGPACPAFSAHSYTSNLGDYDEQALGIMQTLGVDRQRTVESLQNSSYNHFAAIYYLLLERLKEYRNAQCARPGPARQPRPRSSDLSGLEVPQEGLSTDPFRPALLCPQPQTLVQSVLQAEMDCELQSSLQWPLFFPVDASCSGVFRPRPVSPSSLLDTAISEEARQGPGLEEEQDTQESLPSSTGRRHTLAEVSTRLSPLTAPCIVVSPSTTASPAEGTSSDSCLTFSASKSPAGLSGTPATQGLLGACSPVRLASPFLGSQSATPVLQAQGGLGGAVLLPVSFQEGRRASDTSLTQGLKAFRQQLRKTTRTKGFLGLNKIKGLARQVCQVPASRASRGGLSPFHAPAQSPGLHGGAAGSREGWSLLEEVLEQQRLLQLQHHPAAAPGCSQAPQPAPAPFVIAPCDGPGAAPLPSTLLTSGLPLLPPPLLQTGASPVASAAQLLDTHLHIGTGPTALPAVPPPRLARLAPGCEPLGLLQGDCEMEDLMPCSLGTFVLVQ

SEQ ID NO: 21 - DNA, 5749 bp, Human
cgcgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagggatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagcctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttagtgcaaggggaaacctgtgaaagctgcactcagagggaggagtctttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaaggactgtaagcagattacaataaatcctttacttt

SEQ ID NO: 22 - protein, 984 aa, Human
METKGYHSLPEGLDMERRWGQVSQAVERSSLGPTERTDENNYMEIVNVSCVSGAIPNNSTQGSSKEKQELLPCLQQDNNRPGILTSDIKTELESKELSATVAESMGLYMDSVRDADYSYEQQNQQGSMSPAKIYQNVEQLVKFYKGNGHRPSTLSCVNTPLRSFMSDSGSSVNGGVMRAIVKSPIMCHEKSPSVCSPLNMTSSVCSPAGINSVSSTTASFGSFPVHSPITQGTPLTCSPNAENRGSRSHSPAHASNVGSPLSSPLSSMKSSISSPPSHCSVKSPVSSPNNVTLRSSVSSPANINNSRCSVSSPSNTNNRSTLSSPAASTVGSICSPVNNAFSYTASGTSAGSSTLRDVVPSPDTQEKGAQEVPFPKTEEVESAISNGVTGQLNIVQYIKPEPDGAFSSSCLGGNSKINSDSSFSVPIKQESTKHSCSGTSFKGNPTVNPFPFMDGSYFSFMDDKDYYSLSGILGPPVPGFDGNCEGSGFPVGIKQEPDDGSYYPEASIPSSAIVGVNSGGQSFHYRIGAQGTISLSRSARDQSFQHLSSFPPVNTLVESWKSHGDLSSRRSDGYPVLEYIPENVSSSTLRSVSTGSSRPSKICLVCGDEASGCHYGVVTCGSCKVFFKRAVEGQHNYLCAGRNDCIIDKIRRKNCPACRLQKCLQAGMNLGARKSKKLGKLKGIHEEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 23 - DNA, 858 bp, Human
atgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaactcccaaggcccttttgggtccagaagacctgcatatgggctgttgactcatgcaaatgaggtatctgaactgcagcttcagtattagcagagccacaggccgcctctgtggcatcaccagggtttctctgaagaagagggtctgcattttcctaaacccagtgctgctctcccatctcccatcttcctctcgcagcttgatgagccccggtgtgtcccaggtacacccctgcatccaggcagcagcccaggccaccccctcctcactggcccttggctcctttcttgatgcctctgttgcttgtcccccaggagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaa

SEQ ID NO: 24 - protein, 135 aa, Human
MMLQHPGQVSASEVSASAIVPCLSPPGSLVFEDFANLTPFVKEELRFAIQNKHLCHRMSSALESVTVSDRPLGVSITKAEVAPEEDERKKRRRERNKIAAAKCRNKKKEKTECLQKLPRPFWVQKTCIWAVDSCK

SEQ ID NO: 25 - DNA, 2482 bp, Human
gacttccggccgcgcagcggtgggctgagctaaaatggctgaggagagagtcgcgacgagaactcaatttcctgtatctactgagtctcaaaaaccccggcagaaaaaagctccagagtttcctattttggagaagcagaactggttgattcatcttcattatatccggaaagattatgaagcctgcaaggctgttatcaaagaacagcttcaagagactcagggattgtgtgaatatgctatctatgtccaagcattgatatttcgcctagaaggaaatatccaagaatccctagaactcttccagacatgtgcagttcttagtcctcagagtgctgataacctcaagcaggtggccagatctttatttcttttgggaaaacataaagctgccattgaagtatataatgaagcagctaaactcaaccagaaagattgggagatcagccataacctaggagtttgctacatatacctgaagcagttcaacaaggcacaagaccagttgcacaatgccctgaatcttaataggcacgatctgacttatataatgctggggaagatccacttgctggagggagacttggacaaggccattgaagtctacaagaaagcagtggagttctcaccagaaaatacagagcttcttacaactttaggattactctacttacagctcggcatttaccagaaggcatttgaacatcttggcaatgcactgacttatgaccctaccaactacaaggccatcttggcagcaggcagcatgatgcagacccacggggactttgatgttgccctcaccaaatacagagttgtggcttgtgctgttccagaaagtcctccactctggaataacattggaatgtgtttctttggcaagaagaaatatgtggcggccatcagctgcctgaaacgagccaactacttggcacccttcgattggaagattctgtataatttgggccttgtccatttgaccatgcagcagtatgcatcagcttttcattttctcagtgcggccatcaacttccagccaaagatgggggagctctacatgctcttggcagtggctctgaccaatctggaagatatagaaaatgccaagagagcctacgcagaagcagtccacctggataagtgtaaccctttagtaaacctgaactatgctgtgctgctgtacaaccagggcgagaagaagaacgccctggcccaatatcaggagatggagaagaaagtcagcctactcaaggacaatagctctctggaatttgactctgagatggtggagatggctcagaagttgggagctgctctccaggttggggaggcactggtctggaccaaaccagttaaagatcccaaatcaaagcaccagaccacttcaaccagcaaacctgccagtttccagcagcctctgggctctaatcaagctctaggacaggcaatgtcttcagcagctgcatacaggacgctcccctcaggtgctggaggaacatcccagttcacaaagcccccatctcttcctctggagccagagcctgcggtggaatcaagtccaactgaaacatcagaacaaataagagagaaataagaatagaatgaatgaccccaaaatagggttttcttgggcgaggatgtgctggattaggaaaggtgacatgacacaggcagagcagagtggcacccaccacagaatacagtgtgtgttattacgaggagccagcagttgagcctaaggtccttctacctacctggtattggcatttgaggtcggaaaccctctactgccccataagccaggaaaagtgaaaagagaacacagttcctttaagaactggcagcaaggcttgaggccttatgtatgtagctgagtcagcaaggtacatgatgctgtctgctttcaaaaggacttttctctcctagctgactgactccttccttagttcaaggaacagctgagacagacctctgctgagtagctctgtgatgacaaagccttggtttaactgaggtgatcctcaggttgtgaggtttattagtccccaaggcaaacacaaatattagattaataatccaactttaatagtatacatttaaaagaaaaaaaacaaaagccctggaagttgaggccaagcctgctgagtattgcagctgcatttgcccaaagggaatccagaacaagtccctccctgtattttgttcttgagaggggtcagtctagaagctagatcctatcaggatgaggagcagcagcccagggcttgtctggatcagcaccaacgattttaaagaaaaaaggaagagtttcttagatgagtaattgttattgaagatagtcagtgataaccactgaccagatgctatcaatacactatgtgtcctttttagaataaagattacatatcatcattcctttggggaaaattgttattcaggtataaaaacaagagatcataataaaaacctaaaagaacctatg

SEQ ID NO: 26 - protein, 519 aa, Human
MAEERVATRTQFPVSTESQKPRQKKAPEFPILEKQNWLIHLHYIRKDYEACKAVIKEQLQETQGLCEYAIYVQALIFRLEGNIQESLELFQTCAVLSPQSADNLKQVARSLFLLGKHKAAIEVYNEAAKLNQKDWEISHNLGVCYIYLKQFNKAQDQLHNALNLNRHDLTYIMLGKIHLLEGDLDKAIEVYKKAVEFSPENTELLTTLGLLYLQLGIYQKAFEHLGNALTYDPTNYKAILAAGSMMQTHGDFDVALTKYRVVACAVPESPPLWNNIGMCFFGKKKYVAAISCLKRANYLAPFDWKILYNLGLVHLTMQQYASAFHFLSAAINFQPKMGELYMLLAVALTNLEDIENAKRAYAEAVHLDKCNPLVNLNYAVLLYNQGEKKNALAQYQEMEKKVSLLKDNSSLEFDSEMVEMAQKLGAALQVGEALVWTKPVKDPKSKHQTTSTSKPASFQQPLGSNQALGQAMSSAAAYRTLPSGAGGTSQFTKPPSLPLEPEPAVESSPTETSEQIREK

SEQ ID NO: 27 - DNA, 1426 bp, Human
aaagctgcggcggcggttcgcgtttctcgtgtccgcttgactgacagctgcgcggcgggagcgggcggcgcgagcgggaggcggcggcgcagagcttggggcttccttggtcgcacccaccacctgcctgcccactggtcagccttcagggaccctgagcaccgcctggtctctttcctgtggccagcccagaactgaagcgctgcggcatggcgcgcgcctgcctccaggccgtcaagtacctcatgttcgccttcaacctgctcttctggctgggaggctgtggcgtgctgggtgtcggcatctggctggccgccacacaggggagcttcgccacgctgtcctcttccttcccgtccctgtcggctgccaacctgctcatcatcaccggcgcctttgtcatggccatcggcttcgtgggctgcctgggtgccatcaaggagaacaagtgcctcctgctcactttcttcctgctgctgctgctggtgttcctgctggaggccaccatcgccatcctcttcttcgcctacacggacaagattgacaggtatgcccagcaagacctgaagaaaggcttgcacctgtacggcacgcagggcaacgtgggcctcaccaacgcctggagcatcatccagaccgacttccgctgctgtggcgtctccaactacactgactggttcgaggtgtacaacgccacgcgggtacctgactcctgctgcttggagttcagtgagagctgtgggctgcacgcccccggcacctggtggaaggcgccgtgctacgagacggtgaaggtgtggcttcaggagaacctgctggctgtgggcatctttgggctgtgcacggcgctggtgcagatcctgggcctgaccttcgccatgaccatgtactgccaagtggtcaaggcagacacctactgcgcgtaggccgcccaccgcccgcttctctgccaaaaggacgcccacggggagatggccgcacccacagctgcctttcccaccaccagcctcggtgctctgccccatgctgggaggagggagggagggacaggtgcctggagcccccggaaccctgtttctggaaggccctagctcaggtggcttcagggcctccggaccccccctgggaggggtggccacgtgctggctgcggaacccagggcaggggtgggaggggcctccagcactttttatatttacgtattctccaaagcagtgttcacacgggagccagcctgtggcccccagcctcctggaaaacaggttggcgctggaggagccgggtcttggcatcctggaggtggccccactggtcctggtgctccaggcggggccgtggacccctcacctacattccatagtgggcccgtggggctcctggtgcatcttaataaagtgtgagcagcaaaaaaaaaaaaaaaaaaaaa

SEQ ID NO: 28 - protein, 238 aa, Human
MARACLQAVKYLMFAFNLLFWLGGCGVLGVGIWLAATQGSFATLSSSFPSLSAANLLIITGAFVMAIGFVGCLGAIKENKCLLLTFFLLLLLVFLLEATIAILFFAYTDKIDRYAQQDLKKGLHLYGTQGNVGLTNAWSIIQTDFRCCGVSNYTDWFEVYNATRVPDSCCLEFSESCGLHAPGTWWKAPCYETVKVWLQENLLAVGIFGLCTALVQILGLTFAMTMYCQVVKADTYCA

SEQ ID NO: 29 - DNA, 6364 bp, Human
ctgggatagaagcggcaggagcagcgttggcaccggcgaaccatggctgggattttctatttcgccctattttcgtgtctcttcgggatttgcgacgctgtcacaggttccagggtataccccgcgaatgaagttaccttattggattccagatctgttcagggagaacttgggtggatagcaagccctctggaaggagggtgggaggaagtgagtatcatggatgaaaaaaatacaccaatccgaacctaccaagtgtgcaatgtgatggaacccagccagaataactggctacgaactgattggatcacccgagaaggggctcagagggtgtatattgagattaaattcaccttgagggactgcaatagtcttccgggcgtcatggggacttgcaaggagacgtttaacctgtactactatgaatcagacaacgacaaagagcgtttcatcagagagaaccagtttgtcaaaattgacaccattgctgctgatgagagcttcacccaagtggacattggtgacagaatcatgaagctgaacaccgagatccgggatgtagggccattaagcaaaaaggggttttacctggcttttcaggatgtgggggcctgcatcgccctggtatcagtccgtgtgttctataaaaagtgtccactcacagtccgcaatctggcccagtttcctgacaccatcacaggggctgatacgtcttccctggtggaagttcgaggctcctgtgtcaacaactcagaagagaaagatgtgccaaaaatgtactgtggggcagatggtgaatggctggtacccattggcaactgcctatgcaacgctgggcatgaggagcggagcggagaatgccaagcttgcaaaattggatattacaaggctctctccacggatgccacctgtgccaagtgcccaccccacagctactctgtctgggaaggagccacctcgtgcacctgtgaccgaggctttttcagagctgacaacgatgctgcctctatgccctgcacccgtccaccatctgctcccctgaacttgatttcaaatgtcaacgagacatctgtgaacttggaatggagtagccctcagaatacaggtggccgccaggacatttcctataatgtggtatgcaagaaatgtggagctggtgaccccagcaagtgccgaccctgtggaagtggggtccactacaccccacagcagaatggcttgaagaccaccaaagtctccatcactgacctcctagctcataccaattacacctttgaaatctgggctgtgaatggagtgtccaaatataaccctaacccagaccaatcagtttctgtcactgtgaccaccaaccaagcagcaccatcatccattgctttggtccaggctaaagaagtcacaagatacagtgtggcactggcttggctggaaccagatcggcccaatggggtaatcctggaatatgaagtcaagtattatgagaaggatcagaatgagcgaagctatcgtatagttcggacagctgccaggaacacagatatcaaaggcctgaaccctctcacttcctatgttttccacgtgcgagccaggacagcagctggctatggagacttcagtgagcccttggaggttacaaccaacacagtgccttcccggatcattggagatggggctaactccacagtccttctggtctctgtctcgggcagtgtggtgctggtggtaattctcattgcagcttttgtcatcagccggagacggagtaaatacagtaaagccaaacaagaagcggatgaagagaaacatttgaatcaaggtgtaagaacatatgtggacccctttacgtacgaagatcccaaccaagcagtgcgagagtttgccaaagaaattgacgcatcctgcattaagattgaaaaagttataggagttggtgaatttggtgaggtatgcagtgggcgtctcaaagtgcctggcaagagagagatctgtgtggctatcaagactctgaaagctggttatacagacaaacagaggagagacttcctgagtgaggccagcatcatgggacagtttgaccatccgaacatcattcacttggaaggcgtggtcactaaatgtaaaccagtaatgatcataacagagtacatggagaatggctccttggatgcattcctcaggaaaaatgatggcagatttacagtcattcagctggtgggcatgcttcgtggcattgggtctgggatgaagtatttatctgatatgagctatgtgcatcgtgatctggccgcacggaacatcctggtgaacagcaacttggtctgcaaagtgtctgattttggcatgtcccgagtgcttgaggatgatccggaagcagcttacaccaccaggggtggcaagattcctatccggtggactgcgccagaagcaattgcctatcgtaaattcacatcagcaagtgatgtatggagctatggaatcgttatgtgggaagtgatgtcgtacggggagaggccctattgggatatgtccaatcaagatgtgattaaagccattgaggaaggctatcggttaccccctccaatggactgccccattgcgctccaccagctgatgctagactgctggcagaaggagaggagcgacaggcctaaatttgggcagattgtcaacatgttggacaaactcatccgcaaccccaacagcttgaagaggacagggacggagagctccagacctaacactgccttgttggatccaagctcccctgaattctctgctgtggtatcagtgggcgattggctccaggccattaaaatggaccggtataaggataacttcacagctgctggttataccacactagaggctgtggtgcacgtgaaccaggaggacctggcaagaattggtatcacagccatcacgcaccagaataagattttgagcagtgtccaggcaatgcgaacccaaatgcagcagatgcacggcagaatggttcccgtctgagccagtactgaataaactcaaaactcttgaaattagtttacctcatccatgcactttaattgaagaactgcactttttttacttcgtcttcgccctctgaaattaaagaaatgaaaaaaaaaaacaatatctgcagcgttgcttggtgcacagattgctgaaactgtggggcttacagaaatgactgccggtcatttgaatgagacctggaacaaatcgtttctcagaagtacttttctgttcatcaccagtctgtaaaatacatgtacctatagaaatagaacactgcctctgagttttgatgctgtatttgctgccagacactgagcttctgagacatccctgattctctctccatttggaattacaaccattgtattttgtttgtggcataaattacagtcatctgtctttcactggaatgaagaccatgcctaggaacattttttaaggactcagctgtggcttttagggcttggttcataccatgggggaaaaaaaagtcctaggagaaagcgacgtggctcattagtgttgcctcttcagtgctcaagccgcctggtggattcctatgacacagggggcctggaaagaaagggaaagtggatttaaaatatatatatacgtaacccaagccccataacccctaactggacaaatgaggtctgtttctttgggcctgaggctgtgccatataaagtcttattttgggactttacaaacttgtcctaactatcttgtggatagtgggctgtgacaatctggaatagagaacgttcacacttcgctcctttaaagaagcgaccccagatctgcaagggagtagattctgctatcttggcctcacagcccttcctgttgattacaaagcccgtggaagaaaacagaacacaccctcctcagttccgtctaaatgtgtttcttctgcttcaattacaccagttctggggcaaagacactgatgaaacaacacccatacctgaaaagaataaatgtgtgactttcaaatcccctttcgcagtgaaagaaacagcaaacacttaagattcagcatctgttctccagttgcactgaggaatgcactgtctcgcagcaccagctctgcagagcccttgccccagactctttgcggttttatttatatgtatttccatatttcattcctgtgtgtcactgctgcattggtgtggcagcaagtgaccaaatgctacaggtcttactatggacaccaggtcaggtgcaaccacacaaaacaaagccagttccatgagctgcctatgatatgcattgcggaagtaacattttacccagggtgtgccattgcagtgatataaatatatttttttcttagactaaatatgagctgactatctcttttgatgtgtgtacataggtgtgagtgtgtgtgtatgcgtgcctgtctgtgtgcgggtgtgtgtatgtgcatagcctcatgcttaggactacccatgaatgttgtggaatgctacacctggagagttctggttttccaccagtttcaagatgaagaactacatgatacagtggacctggagaccatccccttggaaagacaacccagagatgttcagcatcctgtatctacacgcatcctgtatctacacgtgtattttgtagctgtcacactaaccttaataagaattctacagctttggacagaggcattttcaccttaatggtgaagtaatttaaaatataaatccattcaggtgacaacccatcatcaaaattacaaattttctgattgaactcatctgaatcatcagttccttgatggagagagagaaggagatggaatgtgtctggtaaccccaaatggagtacaagtagcctttgttttcctgcataaatggacttgttgaatgcgaacgaatatatgcaattcatatacttttggagatgaacgtagatatgtgtgtcagctttgagatggtgtgtcctggattaatactttgtctcccaatatcacagaaaaatacatgccagtgactcttgaggttaaggtagttgggatgaaatggcctcaggcaatttcacattccctaattacctggaaagttctacagtaattaatatgcagctaactcctgttgccctcacaagagcatcagccttctagaatcggagctccggagtgtgaagattcagtattgatatgatatgtataccaaactccagccaacttactgccatttttcataatctgagtggctgccttgcttatcctaagctgtggttgcagaaaccgtggccatttatataagctataacatcaaatcagggaaaaatgaggaaaaaaaatagattctgaaccatttattgttgaataagtagagaaaatcatcaataaatatttattacattctgacagggtgtgtggcattgtgttctatgccagagtgacaaagttgattcacccctttttggggaccttaatatattttttaagggatgtgcctatgcattgatgcctgaaaaatatgtataaagaaatgaggttgactcttctgagcagttcatcttttccagaggtaagggtaggaggccaacttcagggtctgggtctgagcccgtgggcaagccctggccgagtgagctccaatgctaactcatgtgccgatctctagagcagtgggaaactaccccgctgcaccaaatcaagtagcttcaccttgtgtatgcaggccccaagttattttttagcaatcttacgagtgaaatgttctggtgggttgaaaaacgttcttattttaaagaaaggttgtgctcgctacactgctggtgtgtgcattctgagacctcttgtattcaatctgtgaaggatatgtgtattaatccgtacacccgtatagcctcaatatttgtctgaagacacttaaattctgacccataaaggaaagttctagaagcaatattttcacttatttaacattctccaaacaacatcaagcattgatacacactgaagagtgcgtttattttttgtatcactctaagtatgttggaatatgcaaggactgtggttcaaattagaatgtataaggcatattataatttagttcatactgaataagaaattaacagaacattgttcggttcacacgttccaaactttgagtgatttctggagttagacatagattttctattttgttttaatttgtcaaggtatttttcttcccttcatgaactttaggtacacataacttatgtcatttatttatggtcttttatacctagtttgtaaaattgtaaaatagcaaactaaatgcaaagagtttgcatttgaaaataataaagtagttgccgtatacaaccctgcaaaaaaaaaaaaaaaaaa

SEQ ID NO: 30 - protein, 986 aa, Human
MAGIFYFALFSCLFGICDAVTGSRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQEDLARIGITAITHQNKILSSVQAMRTQMQQMHGRMVPV

SEQ ID NO: 31 - DNA, 4078 bp, Human
atgcagaagagcgtgcgctacaacgaggggcacgccctgtacctggcctttctggcgcgcaaggagggcaccaagcgcggcttcctgagtaagaagacggccgaggcgagccgctggcacgagaagtggttcgccctctaccagaatgtgctcttctacttcgagggcgagcagagctgccgcccggcgggcatgtacctcctggagggctgcagctgcgaacgaacgcccgcgccacccagggccggcgccgggcagggaggcgtccgagacgcgctggacaagcagtattactttactgttctttttggccatgaaggtcagaagccactggagctgcgctgtgaggaggagcaggatggtaaagagtggatggaggccattcaccaagccagttatgcagacattttgattgagagggaagtattaatgcagaagtacattcatctagttcagatcgtagagacagaaaaaattgcagctaaccaactccgacatcaacttgaagatcaagacacagaaatcgaaaggcttaaatcagagattattgctcttaataaaaccaaagaacgaatgcgaccttaccaaagcaaccaagaagacgaagatccagacatcaagaagattaaaaaggttcagagcttcatgcgaggatggttgtgcagaaggaaatggaagaccatcgtgcaggattacatttgttctcctcatgctgaaagtatgaggaagagaaaccagattgtgttcaccatggtggaggcagagtcagagtacgttcaccagctctacatcctggtcaatggctttctccggcccctgcgtatggccgccagctccaagaagccccccatcagccacgacgacgtcagcagtatttttcttaacagtgaaacaatcatgtttcttcatgaaatatttcatcaaggactaaaggcaaggatagcaaactggcccactttaattttagctgatctgtttgatattttgctccccatgctgaacatttatcaagaatttgtgcgtaatcaccagtacagcctgcaagttctcgccaattgtaagcaaaacagagattttgacaaactcttaaaacagtatgaagccaatccagcctgtgaggggaggatgctggagacattcttgacctatcccatgtttcagatccccagatatatcatcacactccatgagctccttgctcacacaccccatgagcatgtggaaaggaaaagcctggagtttgccaaatcaaagctagaggaactatccagagtaatgcacgatgaagtcagcgacactgaaaacataaggaaaaaccttgccatcgaaagaatgatcgtggagggctgtgacatcttgctggacaccagccaaacgttcatccgccaaggttctcttattcaagtaccttccgttgagagggggaaacttagtaaagttcgcctgggttcgttgtctttgaaaaaggaaggagagagacaatgcttcttatttacaaaacactttttaatatgtacaagaagttcaggagggaagcttcatctgctcaagacaggtggggttctgtctctaatagactgcacattgattgaggagccagatgcaagcgatgatgactctaaaggttctgggcaagtgtttgggcacctggattttaaaatagtggtggagcctcctgacgctgccgccttcactgttgtcttgttagcaccctcacgccaggagaaagctgcctggatgagtgacatcagtcagtgtgtggacaatatacgatgtaatggtttaatgactatagtgtttgaagagaattccaaagtcactgtgccacatatgattaagtctgatgcccgtcttcataaagacgacactgacatttgcttcagtaaaacactcaactcctgcaaagtgccccagatccgttatgccagcgtggagcgcctcttggaacgactgacagacttgcggtttcttagtattgatttcctcaacacctttctgcacacctatcgtattttcactactgccgctgtggtgctggggaaactctccgacatatacaagaggcctttcacctccatccctgtcaggtcattggaattgttttttgctaccagccagaacaacagaggtgaacatttggtggatggcaaatccccacgtctgtgtcgcaaattctcttccccgccaccactggctgtgtccagaacatcttccccagtgagggccagaaagctgtctttgacttctcccttgaactcaaagataggagcattggacctgacaacttccagcagtcccaccaccaccacccagagtcccgctgcgtctccaccaccacacactggtcagataccactggatctcagcagaggcctctcttctccagagcaaagcccgggaacggtagaagagaatgtcgataacccacgcgtggatctgtgtaacaagctaaaacgaagtattcaaaaagcagtcctagagtctgcaccagcggaccgagcaggagtggaaagctcccctgcagcggacaccacagaactttcaccttgcagatccccctcaactcctcggcacctccgctatcgacagcctggaggacagacggcggacaatgcccactgctctgtttcaccggcttctgcttttgcaatagccacagctgcagcaggacatgggagtccaccaggctttaacaacaccgagagaacatgtgataaagagtttattatacggagaacggctaccaatcgagttctgaacgtcctccgtcactgggtctcaaagcacgcacaggatttcgaactcaacaatgaactaaagatgaatgtcctaaatttgctagaagaagttttgcgagacccagaccttcttccccaagaaaggaaagccgccgcgaatatcctcagggccctttcacaagatgaccaagatgacatccacctaaaattagaggatataattcaaatgactgactgcatgaaggccgaatgctttgagtccttgtcggccatggagctggcagaacagatcaccctcctggaccatgtcattttcagaagcattccctacgaggagtttcttgggcaggggtggatgaagctggataaaaacgaaagaactccttacattatgaaaaccagccaacacttcaatgacatgagtaacctggtggcctcccagataatgaactatgctgatgtcagctcccgtgccaacgccatcgagaaatgggtggcagtggcggacatctgccgatgcctgcacaactacaacggcgtgctggagatcacctcggccttaaacagaagtgccatctacaggctgaagaaaacctgggccaaggtctctaagcagacaaaagctctaatggacaaacttcaaaagactgtttcctctgaaggaagatttaaaaatcttagagaaacccttaaaaattgtaaccctcctgcagttccttatcttgggatgtacttgacagacctggcatttattgaagaaggaacaccaaactttactgaggaaggccttgtcaatttctccaaaatgagaatgatatcacacatcatcagagagatacgccagttccagcagacttcctacagaatagatcatcagccaaaggtcgcacagtacttgcttgacaaagaccttatcatagatgaagatacgctatatgagctgtcactaaaaattgaacctcgactccctgcttgaagatctggccttgcccctgagtccacgggatgttcatggaaagcaggacagacagaattgtgtatgccttgcctatcacggtacagcacgaagccaggctcctttctccaccaaagaagatggaaccagactggaattctgtctccagagagaaacccagctgtttgggtcaaagacagatgcttcagacttgggtgggaaggtgaaagatggctatttagaaagctggtggcacgttttacataagggaatgtcagatgggagatgctagttgccattttaacaaagcaggtaaatcggtaaattttaaactctgtccatgttctgttagaactcagggacaaggatccatgaaaaagacctg

SEQ ID NO: 32 - protein, 1237 aa, Human
MQKSVRYNEGHALYLAFLARKEGTKRGFLSKKTAEASRWHEKWFALYQNVLFYFEGEQSCRPAGMYLLEGCSCERTPAPPRAGAGQGGVRDALDKQYYFTVLFGHEGQKPLELRCEEEQDGKEWMEAIHQASYADILIEREVLMQKYIHLVQIVETEKIAANQLRHQLEDQDTEIERLKSEIIALNKTKERMRPYQSNQEDEDPDIKKIKKVQSFMRGWLCRRKWKTIVQDYICSPHAESMRKRNQIVFTMVEAESEYVHQLYILVNGFLRPLRMAASSKKPPISHDDVSSIFLNSETIMFLHEIFHQGLKARIANWPTLILADLFDILLPMLNIYQEFVRNHQYSLQVLANCKQNRDFDKLLKQYEANPACEGRMLETFLTYPMFQIPRYIITLHELLAHTPHEHVERKSLEFAKSKLEELSRVMHDEVSDTENIRKNLAIERMIVEGCDILLDTSQTFIRQGSLIQVPSVERGKLSKVRLGSLSLKKEGERQCFLFTKHFLICTRSSGGKLHLLKTGGVLSLIDCTLIEEPDASDDDSKGSGQVFGHLDFKIVVEPPDAAAFTVVLLAPSRQEKAAWMSDISQCVDNIRCNGLMTIVFEENSKVTVPHMIKSDARLHKDDTDICFSKTLNSCKVPQIRYASVERLLERLTDLRFLSIDFLNTFLHTYRIFTTAAVVLGKLSDIYKRPFTSIPVRSLELFFATSQNNRGEHLVDGKSPRLCRKFSSPPPLAVSRTSSPVRARKLSLTSPLNSKIGALDLTTSSSPTTTTQSPAASPPPHTGQIPLDLSRGLSSPEQSPGTVEENVDNPRVDLCNKLKRSIQKAVLESAPADRAGVESSPAADTTELSPCRSPSTPRHLRYRQPGGQTADNAHCSVSPASAFAIATAAAGHGSPPGFNNTERTCDKEFIIRRTATNRVLNVLRHWVSKHAQDFELNNELKMNVLNLLEEVLRDPDLLPQERKAAANILRALSQDDQDDIHLKLEDIIQMTDCMKAECFESLSAMELAEQITLLDHVIFRSIPYEEFLGQGWMKLDKNERTPYIMKTSQHFNDMSNLVASQIMNYADVSSRANAIEKWVAVADICRCLHNYNGVLEITSALNRSAIYRLKKTWAKVSKQTKALMDKLQKTVSSEGRFKNLRETLKNCNPPAVPYLGMYLTDLAFIEEGTPNFTEEGLVNFSKMRMISHIIREIRQFQQTSYRIDHQPKVAQYLLDKDLIIDEDTLYELSLKIEPRLPA

SEQ ID NO: 33 - DNA, 4703 bp, Human
cgccgatggtacgcgccggctcgcctggcccggctgcagtggatgttgctagaacccacgcggaggaaggaagagacgcaggcaggctgcggttacccaagcggccacccgggcctcagggaccccttccccgagagacggcaccatgacccagggaaagctctccgtggctaacaaggcccctgggaccgaggggcagcagcaggtgcatggcgagaagaaggaggctccagcagtgccctcagccccaccctcctatgaggaagccacctctggggaggggatgaaggcaggggccttccccccagcccccacagcggtgcctctccaccctagctgggcctatgtggaccccagcagcagctccagctatgacaacggtttccccaccggagaccatgagctcttcaccactttcagctgggatgaccagaaagttcgtcgagtctttgtcagaaaggtctacaccatcctgctgattcagctgctggtgaccttggctgtcgtggctctctttactttctgtgaccctgtcaaggactatgtccaggccaacccaggctggtactgggcatcctatgctgtgttctttgcaacctacctgaccctggcttgctgttctggacccaggaggcatttcccctggaacctgattctcctgaccgtctttaccctgtccatggcctacctcactgggatgctgtccagctactacaacaccacctccgtgctgctgtgcctgggcatcacggcccttgtctgcctctcagtcaccgtcttcagcttccagaccaagttcgacttcacctcctgccagggcgtgctcttcgtgcttctcatgactcttttcttcagcggactcatcctggccatcctcctacccttccaatatgtgccctggctccatgcagtttatgcagcactgggagcgggtgtatttacattgttcctggcacttgacacccagttgctgatgggtaaccgacgccactcgctgagccctgaggagtatatttttggagccctcaacatttacctagacatcatctatatcttcaccttcttcctgcagctttttggcactaaccgagaatgaggagccctccctgccccaccgtcctccagagaatgcgcccctcctggttccctgtccctcccctgcgctcctgcgagaccagatataaaactagctgccaacccagcctgtggccaggtcactgtctaccccagcccagcccagccctctgccgcttgtacatacgccatggggaccctgaggaactgaggccacgtcaatccctgtgccgccccattcgcccgttacatcttccaaactgggacggtcaaggctgaaggctcctctgggtttgagggtccaagggacaaggaggagaagcctagcaggatttcagatgcaggagagagacccaggaagcccggcagagccctgagccccactgcaattcctcctagggctgcacaatcatgtggccttagggcacactgtcctgcatccagtctgtgtcctcctgtctttctcatccaggtcaggcattgacactttgtaagaaagggggtaggggacacagctgggcaggtggagtgggtgggcaggatggctgtcccagtctgcccatcttctcttggctctgggaccagcggcttgttctagggatttggacctggaggccaagggcaataggagagggtctgaagcctgtgctgtctgctgcttgctgtgaatggccctcccgggtcatgacagagctcttttggggcaggaggtgagggcagggggccccgctccttggtaagggcctgccctggggctcccagggaagtgggagctggggagccaatccacccagacccgcgtccacctgggaggcatttggggttgcaggaccgagacccacatcctctcactcacttctccacccgccaggcagctgccacaggcctaaggctgagtgaggtgtgattgggccctccggctggatgttctgggggaggaggcaatcgctgtagctgccaggccagccccatctggaaggagtccctgctctgcctcagccctaacccgaggctgtgtgggagtcccttctgcacctcttatttcctaaacccttcctgtcagctgctgtttgctggctcgtccccagccaggcctcctgaggtcttcatagttggcacagtccacatctcttctagcttcactggccactcactcagcccagagtccctaagtgctcccccacttatctctgcatctctccaatttggaagccatccagctgatgctaacagccagggccagtccctccagctggtcccatgtaatgaggccagggctccttatcagcttccaggcggctgggatctcaggcagggtgtgcgtcagccctccaacccccagaggggcgcctgtggggagggaaccaggaggccacactgggggagcgtgcagccagcatgtgcagtggccaactccaggccagtggaaagttactgtgtggatacaggaaggctctgagtggctagcaggaccctcccttggcttcgccctcctcctcttctctctcccctgtgctgggagctccgtctgccttgggaagggagacgaggtgtgctgttccttcccagccccatccctccccacccttcctactcccagctttcaagaaacattgcagaagcatttgaagttttgttccatcttggatatttgaagagaaaaggagccaagaactggcgggtggtgtgtgtgcagcaaaggcattgggggcacgggtgagatgcccactgtgtgctggtctaaattaatccatcgcagtgatcctccagagacagcgctctgctcatgtccgtagcagagggaaaagccgaggtgctcagaaaggctcggtaatgagtcccaggtcatacggctagcgagtggcgggcctggggttcagacccacagctgcctcaagcaaacagggtatcacgcttgcccctgggcctgggtgggggccggaatgagaccacatgagagcgcagagaggtgggtggtcaggccctgatgtcctggggatttcttgtcctcagtgccacccggcctcccgtctttcctgaaacccctcgccagattttgcactctgaggtttctatcttccagacaagagctgggaggcaagatttctggtttctggttcctagcctcactgccagcgggctcctgctattaggtgagggtttcttgctgtctggaattcagtttttccatctgccacatggaagttcatagccaggatagggcgggagaggcaccagaacatttctctccactgactttctcacccggttcccaccctcctccacctcctctcctccgggcccctcagtttcctctcctcatctctggcctcagttccagagggttagccccagcaccccactctcctgccactgtcagatagccttggtcatgcttcctagaatctacttgtccaagttctcctctccctgggataagttactgcagaaactggggatttagaagcttattcagggctgagtgccaaaagcttgggggaggagggacaggtcctcggcaataaacagtgtcacacacgcatcacagccatttgagaaatggcttctttgcagagctggggaagccgggcagaagatgccgcagcctcctactctgggtcctgagggggggtggcaggtaccacagtgtaggcagcagggtcagggccaaagtctatgcgttccatctccaatccaggcagtgaggaatcctgaaacaccaggagaggcgtcagaagtaggaagggcaggggaacaggaatgcaagattaaggaaaccgcagctgagcaggtgtcacaagctgtaaaggaggaagggagtctgcaggcccttcacatttttggcctgggtggggctgggcccaggaaggggtgggctggattttgtaaagctggaggcagccaggaatctgactgtccggaatgtgagttgaggggtctcccactgaagtcctcttctggaagctgcagatgggggcccgggaggcacacacgagtctgggactcattcccgggcacacctatggctccttcggtatcacctggggaagccccggaatcttagttcttccattccctactcttccttccgagggagtcaggatgggacagacacccattttccaaaaggctccttgcctccttccctggcccacctgaagccaagtcccaggtgtcctggctgctctcctgggcggggtgggatttttgcataagcactttgctgccctccagtggctgcttgaggaaacgcagggcctggtttgggagagtcaccgagggcccatgtggctggaggttgggggcaggatctggcccgagctctcctcagacctccccagacctgtcttctcatcagccaccgctgttttcttttgccgaacgatggttgcatctctcggacacagtgtaccccctcccaagccctccctgagccgttctcctgagtccctgagggcagacacagctccagggacccaggccagctgccacacccccgacactcctgctgtccactggccttagctcctgcaagttccccagtgtccgcacctgttcccttccctgacctcaccttcctggggtctgtgactgactggagataaaaataaaagcaattgtgacacctg

SEQ ID NO: 34 - protein, 316 aa, Human
MTQGKLSVANKAPGTEGQQQVHGEKKEAPAVPSAPPSYEEATSGEGMKAGAFPPAPTAVPLHPSWAYVDPSSSSSYDNGFPTGDHELFTTFSWDDQKVRRVFVRKVYTILLIQLLVTLAVVALFTFCDPVKDYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQYVPWLHAVYAALGAGVFTLFLALDTQLLMGNRRHSLSPEEYIFGALNIYLDIIYIFTFFLQLFGTNRE

SEQ ID NO: 35 - DNA, 2032 bp, Human
gtggccagggcgcgagagtgcaacgtcctcctggccccgagcgcgtcgtcgcgccccgggagcagaccctcgcccagcagttaccgccgtcccgactttccgttccagttgcagctcctgccgggcaacatgtcaagagccgccgccgctacagctgccgccgccacctggggaagagcagcagcagcagcggcggccgcgggcacacgggggcaataaaccgagccacccgggcgtccagcgtgccggggaaccctctctgcgctcactgcccggcgggacccacgccatgtgctgagccatgtccctggccgcgcccgcgggcagcgcatggggcagcgcctgagtggcggcagatcttgcctcgatgtccccggccggctcctaccgcagccgccgccgcccccgccgccggtgaggaggaagctcgcgctgctcttcgccatgctctgcgtctggctctatatgttcctgtactcgtgcgccggctcctgcgccgccgcgccggggctgctgctcctgggctctgggtcccgcgccgcacacgacccgccagccctggccacagctccggacgggacgccccccaggctgccgttccgggcgccgccagccaccccactggcttcaggcaaggagatggccgagggcgctgcgagcccggaggagcagagtcccgaggtgccggactccccaagccccatctccagctttttcagtgggtctgggagcaagcagctgccgcaggccatcatcatcggcgtgaagaagggcggcacgcgggcgctgctggagtttctgcgcgtgcaccccgacgtgcgcgccgtgggcgccgagccccatttcttcgatcgcagctacgacaagggcctcgcttggtaccgggacctgatgcccagaaccctggacgggcagatcaccatggagaagacgcccagttacttcgtcacgcgggaggcccccgcgcgcatctcggccatgtccaaggacaccaagctcatcgtggtggtgcgggacccggtgaccagggccatctcggactacacgcagacgctgtccaagcggcccgacatccccaccttcgagagcttgacgttcaaaaacaggacagcgggcctcatcgacacgtcgtggagcgccatccagatcggcatctacgccaagcacctggagcactggctgcgccacttccccatccgccagatgctcttcgtgagcggcgagcggctcatcagcgacccggccggggagctgggccgcgtgcaagacttcctgggcctcaagaggatcatcacggacaagcacttctacttcaacaagaccaagggcttcccctgcctgaagaaggcggagggcagcagccggccccattgcctgggcaagaccaagggcaggacccatcctgagatcgaccgcgaggtggtgcgcaggctgcgcgagttctaccggcctttcaacctcaagttctaccagatgaccgggcacgactttggctgggattgagcagacccgggctatgtaccttacccacgtggcttatctattgacagagattatatgtatgtaaaatgtacagaaatctattttataataatttatttttaattcataagcaattaattcactaagctgcctagccacactctttagagagttagcttcataatctgttaacattccaaagtgtttaactctagtatttcgttttcttcttcacaattgatggtgcttctattttttcttctcccctacctgttatatttaaaacaaagaaaagcacaacttgagatttttgttgttacgggtattcagccttcagtcaccgtctgagttctccagttgctgcctccttgtcttgtcttgggtctcccattccagcttccctgtctcttcctgcctgtgtacctcgtaggaacgctgagctgcctcaacagggctgtattctgaagggcaggcctcatgcagcagcctccttgcagatgtggtgtcccgtccaatgatgtagcctgaaagccacagccctagggttctg

SEQ ID NO: 36 - protein, 390 aa, Human
MGQRLSGGRSCLDVPGRLLPQPPPPPPPVRRKLALLFAMLCVWLYMFLYSCAGSCAAAPGLLLLGSGSRAAHDPPALATAPDGTPPRLPFRAPPATPLASGKEMAEGAASPEEQSPEVPDSPSPISSFFSGSGSKQLPQAIIIGVKKGGTRALLEFLRVHPDVRAVGAEPHFFDRSYDKGLAWYRDLMPRTLDGQITMEKTPSYFVTREAPARISAMSKDTKLIVVVRDPVTRAISDYTQTLSKRPDIPTFESLTFKNRTAGLIDTSWSAIQIGIYAKHLEHWLRHFPIRQMLFVSGERLISDPAGELGRVQDFLGLKRIITDKHFYFNKTKGFPCLKKAEGSSRPHCLGKTKGRTHPEIDREVVRRLREFYRPFNLKFYQMTGHDFGWD

SEQ ID NO: 37 - DNA, 3697 bp, Human
cccacgcgtccgggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaattttttttttttttttttaagaatatctgtaatggtactgactttgcttgctttgaagtagctcttttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcaaaaaaaaaaaaaaaaaaaaaaaaaaaaaaaaa

SEQ ID NO: 38 - protein, 781 aa, Human
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 39 - DNA, 1735 bp, Human
caaaatcccaggcagcatggacctcagtcttctctgggtacttctgcccctagtcaccatggcctggggccagtatggcgattatggatacccataccagcagtatcatgactacagcgatgatgggtgggtgaatttgaaccggcaaggcttcagctaccagtgtccccaggggcaggtgatagtggccgtgaggagcatcttcagcaagaaggaaggttctgacagacaatggaactacgcctgcatgcccacgccacagagcctcggggaacccacggagtgctggtgggaggagatcaacagggctggcatggaatggtaccagacgtgctccaacaatgggctggtggcaggattccagagccgctacttcgagtcagtgctggatcgggagtggcagttttactgttgtcgctacagcaagaggtgcccatattcctgctggctaacaacagaatatccaggtcactatggtgaggaaatggacatgatttcctacaattatgattactatatccgaggagcaacaaccactttctctgcagtggaaagggatcgccagtggaagttcataatgtgccggatgactgaatacgactgtgaatttgcaaatgtttagatttgccacataccaaatctgggtgaaaggaaaggggccggggacaggagggtgtccacatatgttaacatcagttggatctcctatagaagtttctgctgctctctttccttctccctgagctggtaactgcaatgccaacttcctgggcctttctgactagtatcacacttctaataaaatccacaattaaaccatgtttctcacttttcacatgtttcatagcaactgctttatatgactgatgatggcttccttgcacaccacatatacagtgcgcatgcttacagccgggcttctggagcaccagctgcagcctggctactgctttttactgcagaatgaactgcaagttcagcatagtggaggggagaggcagaactggaggagaggtgcagtgaaggttctctacagctaagcctgtttgaatgatacgtaggttccccaccaaaagcaggctttctgccctgagggacatcttcccactcccctgctccacatgagccatgcatgcttagcaatccaagtgcagagctctttgctccaggagtgaggagactgggaggtgaaatggggaaatggaagggtttggaggcagagctgaaaacagggttggaaggatttcctgaattagaagacaaacgttagcatacccagtaaggaaaatgagtgcaggggccaggggaacccgtgaggatcactctcaaatgagattaaaaacaaggaagcagagaatggtcagagaatgggattcagattgggaacttgtggggatgagagtgaccaggttgaactgggaagtggaaaaaggagtttgagtcactggcacctagaagcctgcccacgattcctaggaaggctggcagacaccctggaaccctggggagctactggcaaactctcctggattgggcctgatttttttggtgggaaaggctgccctggggatcaactttccttctgtgtgtggctcaggagttcttctgcagagatggcgctatctttcctcctcctgtgatgtcctgctcccaaccatttgtactcttcattacaaaagaaataaaaatattaacgttcactaaaaaaaaaaaaaaaaaaaaaaaaaaaaaaaaa

SEQ ID NO: 40 - protein, 201 aa, Human
MDLSLLWVLLPLVTMAWGQYGDYGYPYQQYHDYSDDGWVNLNRQGFSYQCPQGQVIVAVRSIFSKKEGSDRQWNYACMPTPQSLGEPTECWWEEINRAGMEWYQTCSNNGLVAGFQSRYFESVLDREWQFYCCRYSKRCPYSCWLTTEYPGHYGEEMDMISYNYDYYIRGATTTFSAVERDRQWKFIMCRMTEYDCEFANV

SEQ ID NO: 41 - DNA, 3360 bp, HUMAN
gaattccggctgtgccgcaccgaggcgagcaggagcagggaacaggtgtttaaaattatccaactgccatagagctaaattcttttttggaaaattgaaccgaacttctactgaatacaagatgaaaatgtggttgctggtcagtcatcttgtgataatatctattactacctgtttagcagagtttacatggtatagaagatatggtcatggagtttctgaggaagacaaaggatttggaccaatttttgaagagcagccaatcaataccatttatccagaggaatcactggaaggaaaagtctcactcaactgtagggcacgagccagccctttcccggtttacaaatggagaatgaataatggggacgttgatctcacaagtgatcgatacagtatggtaggaggaaaccttgttatcaacaaccctgacaaacagaaagatgctggaatatactactgtttagcatctaataactacgggatggtcagaagcactgaagcaaccctgagctttggatatcttgatcctttcccacctgaggaacgtcctgaggtcagagtaaaagaagggaaaggaatggtgcttctctgtgaccccccataccattttccagatgatcttagctatcgctggcttctaaatgaatttcctgtatttatcacaatggataaacggcgatttgtgtctcagacaaatggcaatctctacattgcaaatgttgaggcttccgacaaaggcaattattcctgctttgtttccagtccttctattacaaagagcgtgttcagcaaattcatcccactcattccaatacctgaacgaacaacaaaaccatatcctgctgatattgtagttcagttcaaggatgtatatgcattgatgggccaaaatgtgaccttagaatgttttgcacttggaaatcctgttccggatatccgatggcggaaggttctagaaccaatgccaagcactgctgagattagcacctctggggctgttcttaagatcttcaatattcagctagaagatgaaggcatctatgaatgtgaggctgagaacattagaggaaaggataaacatcaagcaagaatttatgttcaagcattccctgagtgggtagaacacatcaatgacacagaggtggacataggcagtgatctctactggccttgtgtggccacaggaaagcccatccctacaatccgatggttgaaaaatggatatgcgtatcataaaggggaattaagactgtatgatgtgacttttgaaaatgccggaatgtatcagtgcatagctgaaaacacatatggagccatttatgcaaatgctgagttgaagatcttggcgttggctccaacttttgaaatgaatcctatgaagaaaaagatcctggctgctaaaggtggaagggtgataattgaatgcaaacctaaagctgcaccgaaaccaaagttttcatggagtaaagggacagagtggcttgtcaatagcagcagaatactcatttgggaagatggtagcttggaaatcaacaacattacaaggaatgatggaggtatctatacatgctttgcagaaaataacagagggaaagctaatagcactggaacccttgttatcacagatcctacgcgaattatattggccccaattaatgccgatatcacagttggagaaaacgccaccatgcagtgtgctgcgtcctttgatcctgccttggatctcacatttgtttggtccttcaatggctatgtgatcgattttaacaaagagaatattcactaccagaggaattttatgctggattccaatggggaattactaatccgaaatgcgcagctgaaacatgctggaagatacacatgcactgcccagacaattgtggacaattcttcagcttcagctgaccttgtagtgagaggccctccaggccctccaggtggtctgagaatagaagacattagagccacttctgtggcacttacttggagccgtggttcagacaatcatagtcctatttctaaatacactatccagaccaagactattctttcagatgactggaaagatgcaaagacagatcccccaattattgaaggaaatatggaggcagcaagagcagtggacttaatcccatggatggagtatgaattccgcgtggtagcaaccaatacactgggtagaggagagcccagtataccatctaacagaattaaaacagacggtgctgcaccaaatgtggctccttcagatgtaggaggtggaggtggaagaaacagagagctgaccataacatgggcgcctttgtcaagagaataccactatggcaacaattttggttacatagtggcatttaagccatttgatggagaagaatggaaaaaagtcacagttactaatcctgatactggccgatatgtccataaagatgaaaccatgagcccttccactgcatttcaagttaaagtcaaggccttcaacaacaaaggagatggaccttacagcctactagcagtcattaattcagcacaagacgctcccagtgaagccccaacagaagtaggtgtaaaagtcttatcatcttctgagatatctgttcattgggaacatgttttagaaaaaatagtggaaagctatcagattcggtattgggctgcccatgacaaagaagaagctgcaaacagagttcaagtcaccagccaagagtactcggccaggctcgagaaccttctgccagacacccagtattttatagaagtcggggcctgcaatagtgcagggtgtggacctccaagtgacatgattgaggctttcaccaagaaagcacctcctagccagcctccaaggatcatcagttcagtaaggtctggttcacgctatataatcacctgggatcatgtcgttgcactatcaaatgaatctacagtgacgggatataaggtactctacagacctgatggccagcatgatggcaagctgtattcaactcacaaacactccatagaagtcccaatccccagagatggagaatacgttgtggaggttcgcgcgcacagtgatggaggagatggagtggtgtctcaagtcaaaatttcaggtgcacccaccctatccccaagtcttctcggcttactgctgcctgcctttggcatccttgtctacttggaattctgaatgtgttgtgacagctgctgttcccatcccagctcagaagacacccttcaaccctgggatgaccacaattccttccaatttctgcggctccatcctaagccaaataaattatactttaacaaactattcaactgatttacaacacacatgatgactgaggcattcaggaaccccttcatcca

SEQ ID NO: 42 - protein, 1018 aa, HUMAN [sequence omitted]

SEQ ID NO: 43 - DNA, 3982 bp, HUMAN
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacaatttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaagtatgtattcttcactactgagtttcttctttggaaaccatcactattgagagatgggaaaaacctgaatgtataaagcatttatttgtcaataaactgccttttgtaaggggttttcacataaaaaaaaaaaaa

SEQ ID NO: 44 - protein, 982 aa, Human [sequence omitted]

SEQ ID NO: 45 - DNA, 4046 bp, Human
gcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttagcaagagatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcactgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcttcaagcctgaggttttatatacaaactccctgaatactctttttgccttgtatcttctcagcctcctagccaagtcctatgtaatatggaaaacaaacactgcagacttgagattcagttgccgatcaaggctctggcattcagagaacccttgcaactcgagaagctgtttttatttcgtttttgttttgatccagtgctctcccatctaacaactaaacaggagccatttcaaggcgggagatattttaaacacccaaaatgttgggtctgattttcaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccagcaaacactgaatttctcttgttgttttaactctgccacaagaatgcaatttcgttaacggagatgacttaagttggcagcagtaatcttcttttaggagcttgtaccacagtcttgcacataagtgcagatttggctcaagtaaagagaatttcctcaacactaacttcactgggataatcagcagcgtaactaccctaaaagcatatcactagccaaagagggaaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagatatttgtggtcttgatcatacctattaaaataatgccaaacaccaaatatgaattttatgatgtacactttgtgcttggcattaaaagaaaaaaacacacatcctggaagtctgtaagttgttttttgttactgtaggtcttcaaagttaagagtgtaagtgaaaaatctggaggagaggataatttccactgtgtggaatgtgaatagttaaatgaaaagttatggttatttaatgtaattattacttcaaatcctttggtcactgtgatttcaagcatgttttctttttctcctttatatgactttctctgagttgggcaaagaagaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaaagagcaactaataaattaaacctattctttcaaaaaaaaa

SEQ ID NO: 46 - protein, 184 aa, Human
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 47 - DNA, 10220 bp, Human
ggaaactctgaaagaacttagaatcagcattttgagagcagaagcttgggcatgctgtgattttccaataaactgctatcacaatgtcaaaatgcagttcagacaagagcaacacagagatctcaaacattaaaacgtaagctgtgctagaacaaaaatgcaatgaaagaaacactggatgaatgaaaagccctgctttgcaacccctcagcatggcaggcctgcagctcatgacccctgcttcctcaccaatgggtcctttctttggactgccatggcaacaagaagcaattcatgataacatttatacgccaagaaaatatcaggttgaactgcttgaagcagctctggatcataataccatcgtctgtttaaacactggctcagggaagacatttattgcagtactactcactaaagagctgtcctatcagatcaggggagacttcagcagaaatggaaaaaggacggtgttcttggtcaactctgcaaaccaggttgctcaacaagtgtcagctgtcagaactcattcagatctcaaggttggggaatactcaaacctagaagtaaatgcatcttggacaaaagagagatggaaccaagagtttactaagcaccaggttctcattatgacttgctatgtcgccttgaatgttttgaaaaatggttacttatcactgtcagacattaaccttttggtgtttgatgagtgtcatcttgcaatcctagaccacccctatcgagaaattatgaagctctgtgaaaattgtccatcatgtcctcgcattttgggactaactgcttccattttaaatgggaaatgtgatccagaggaattggaagaaaagattcagaaactagagaaaattcttaagagtaatgctgaaactgcaactgacctggtggtcttagacaggtatacttctcagccatgtgagattgtggtggattgtggaccatttactgacagaagtgggctttatgaaagactgctgatggaattagaagaagcacttaattttatcaatgattgtaatatatctgtacattcaaaagaaagagattctactttaatttcgaaacagatactatcagactgtcgtgccgtattggtagttctgggaccctggtgtgcagataaagtagctggaatgatggtaagagaactacagaaatacatcaaacatgagcaagaggagctgcacaggaaatttttattgtttacagacactttcctaaggaaaatacatgcactatgtgaagagcacttctcacctgcctcacttgacctgaaatttgtaactcctaaagtaatcaaactgctcgaaatcttacgcaaatataaaccatatgagcgacagcagtttgaaagcgttgagtggtataataatagaaatcaggataattatgtgtcatggagtgattctgaggatgatgatgaggatgaagaaattgaagaaaaagagaagccagagacaaattttccttctccttttaccaacattttgtgcggaattatttttgtggaaagaagatacacagcagttgtcttaaacagattgataaaggaagctggcaaacaagatccagagctggcttatatcagtagcaatttcataactggacatggcattgggaagaatcagcctcgcaacaaacagatggaagcagaattcagaaaacaggaagaggtacttaggaaatttcgagcacatgagaccaacctgcttattgcaacaagtattgtagaagagggtgttgatataccaaaatgcaacttggtggttcgttttgatttgcccacagaatatcgatcctatgttcaatctaaaggaagagcaagggcacccatctctaattatataatgttagcggatacagacaaaataaaaagttttgaagaagaccttaaaacctacaaagctattgaaaagatcttgagaaacaagtgttccaagtcggttgatactggtgagactgacattgatcctgtcatggatgatgatgacgttttcccaccatatgtgttgaggcctgacgatggtggtccacgagtcacaatcaacacggccattggacacatcaatagatactgtgctagattaccaagtgatccgtttactcatctagctcctaaatgcagaacccgagagttgcctgatggtacattttattcaactctttatctgccaattaactcacctcttcgagcctccattgttggtccaccaatgagctgtgtacgattggctgaaagagttgtagctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagctgaaaccgctttttaaaattcaaaacaagaaacaaaacaaaaaaaattaaggggaaaattatttaaatcggaaaggaagacttaaagttgttagtgagtggaatgaattgaaggcagaatttaaagtttggttgataacaggatagataacagaataaaacatttaacatatgtataaaattttggaactaattgtagttttagttttttgcgcaaacacaatcttatcttctttcctcacttctgctttgtttaaatcacaagagtgctttaatgatgacatttagcaagtgctcaaaataattgacaggttttgtttttttttttttgagtttatgtcagctttgcttagtgttagaaggccatggagcttaaacctccagcagtccctaggatgatgtagattcttctccatctctccgtgtgtgcagtagtgccagtcctgcagtagttgataagctgaatagaaagataaggttttcgagaggagaagtgcgccaatgttgtcttttctttccacgttatactgtgtaaggtgatgttcccggtcgctgttgcacctgatagtaagggacagatttttaatgaacattggctggcatgttggtgaatcacattttagttttctgatgccacatagtcttgcataaaaaagggttcttgccttaaaagtgaaaccttcatggatagtctttaatctctgatctttttggaacaaactgttttacattcctttcattttattatgcattagacgttgagacagcgtgatacttacaactcactagtatagttgtaacttattacaggatcatactaaaatttctgtcatatgtatactgaagacattttaaaaaccagaatatgtagtctacggatattttttatcataaaaatgatctttggctaaacaccccattttactaaagtcctcctgccaggtagttcccactgatggaaatgtttatggcaaataattttgccttctaggctgttgctctaacaaaataaaccttagacatatcacacctaaaatatgctgcagattttataattgattggttacttatttaagaagcaaaacacagcacctttacccttagtctcctcacataaatttcttactatacttttcataatgttgcatgcatatttcacctaccaaagctgtgctgttaatgccgtgaaagtttaacgtttgcgataaactgccgtaattttgatacatctgtgatttaggtcattaatttagataaactagctcattatttccatctttggaaaaggaaaaaaaaaaaaacttctttaggcatttgcctaagtttctttaattagacttgtaggcactcttcacttaaatacctcagttcttcttttcttttgcatgcatttttcccctgtttggtgctatgtttatgtattatgcttgaaattttaatttttttttttttgcactgtaactataatacctcttaatttacctttttaaaagctgtgggtcagtcttgcactcccatcaacataccagtagaggtttgctgcaatttgccccgttaattatgcttgaagtttaagaaagctgagcagaggtgtctcatatttcccagcacatgattctgaacttgatgcttcgtggaatgctgcatttatatgtaagtgacatttgaatactgtccttcctgctttatctgcatcatccacccacagagaaatgcctctgtgcgagtgcaccgacagaaaactgtcagctctgctttctaaggaaccctgagtgaggggggtattaagcttctccagtgttttttgttgtctccaatcttaaacttaaattgagatctaaattattaaacgagtttttgagcaaattaggtgacttgttttaaaaatatttaattccgatttggaaccttagatgtctatttgattttttaaaaaaccttaatgtaagatatgaccagttaaaacaaagcaattcttgaattatataactgtaaaagtgtgcagttaacaaggctggatgtgaattttattctgagggtgatttgtgatcaagtttaatcacaaatctcttaatatttataaactacctgatgccaggagcttagggctttgcattgtgtctaatacattgatcccagtgttacgggattctcttgattcctggcaccaaaatcagattgttttcacagttatgattcccagtgggagaaaaatgcctcaatatatttgtaaccttaagaagagtatttttttgttaatactaagatgttcaaacttagacatgattaggtcatacattctcaggggttcaaatttccttctaccattcaaatgttttatcaacagcaaacttcagccgtttcactttttgttggagaaaaatagtagattttaatttgactcacagtttgaagcattctgtgatcccctggttactgagttaaaaaataaaaaagtacgagttagacatatgaaatggttatgaacgcttttgtgctgctgatttttaatgctgtaaagttttcctgtgtttagcttgttgaaatgttttgcatctgtcaattaaggaaaaaaaaaatcactctatgttgccccactttagagccctgtgtgccaccctgtgttcctgtgattgcaatgtgagaccgaatgtaatatggaaaacctaccagtggggtgtggttgtgccctgagcacgtgtgtaaaggactggggaggcgtgtcttgaaaaagcaactgcagaaattccttatgatgattgtgtgcaagttagttaacatgaaccttcatttgtaaattttttaaaatttcttttataatatgctttccgcagtcctaactatgctgcgttttataatagctttttcccttctgttctgttcatgtagcacagataagcattgcacttggtaccatgctttacctcatttcaagaaaatatgcttaacagagaggaaaaaaatgtggtttggccttgctgctgttttgatttatggaatttgaaaaagataattataatgcctgcaatgtgtcatatactcgcacaacttaaataggtcatttttgtctgtggcatttttactgtttgtgaaagtatgaaacagatttgttaactgaactcttaattatgtttttaaaatgtttgttatatttcttttcttttttcttttatattacgtgaagtgatgaaatttagaatgacctctaacactcctgtaattgtcttttaaaatactgatatttttatttgttaataatactttgccctcagaaagattctgataccctgccttgacaacatgaaacttgaggctgctttggttcatgaatccaggtgttcccccggcagtcggcttcttcagtcgctccctggaggcaggtgggcactgcagaggatcactggaatccagatcgagcgcagttcatgcacaaggccccgttgatttaaaatattggatcttgctccgttagggtgcctaatccctttacacaagattgaagccaccaaactgagaccttgatacctttttttaactgcatctgaaattatgttaagagtctttaacccatttgcattatctgcagaagagaaactcatgtcatgtttattacctatatggttgttttaattacatttgaataattatatttttccaaccactgattacttttcaggaatttaattatttccagataaatttctttattttatattgtacatgaaaagttttaaagatatgtttaagaccaagactattaaaatgatttttaaagttgttggagacgccaatagcaatatctaggaaatttgcattgagaccattgtattttccactagcagtgaaaatgatttttcacaactaacttgtaaatatattttaatcattacttctttttttctagtccatttttatttggacatcaaccacagacaatttaaattttatagatgcactaagaattcactgcagcagcaggttacatagcaaaaatgcaaaggtgaacaggaagtaaatttctggcttttctgctgtaaatagtgaaggaaaattactaaaatcaagtaaaactaatgcatattatttgattgacaataaaatatttaccatcacatgctgcagctgttttttaaggaacatgatgtcattcattcatacagtaatcatgctgcagaaatttgcagtctgcaccttatggatcacaattacctttagttgttttttttgtaataattgtagccaagtaaatctccaataaagttatcgtctgttc

SEQ ID NO: 48 - protein, 1922 aa, Human
MKSPALQPLSMAGLQLMTPASSPMGPFFGLPWQQEAIHDNIYTPRKYQVELLEAALDHNTIVCLNTGSGKTFIAVLLTKELSYQIRGDFSRNGKRTVFLVNSANQVAQQVSAVRTHSDLKVGEYSNLEVNASWTKERWNQEFTKHQVLIMTCYVALNVLKNGYLSLSDINLLVFDECHLAILDHPYREIMKLCENCPSCPRILGLTASILNGKCDPEELEEKIQKLEKILKSNAETATDLVVLDRYTSQPCEIVVDCGPFTDRSGLYERLLMELEEALNFINDCNISVHSKERDSTLISKQILSDCRAVLVVLGPWCADKVAGMMVRELQKYIKHEQEELHRKFLLFTDTFLRKIHALCEEHFSPASLDLKFVTPKVIKLLEILRKYKPYERQQFESVEWYNNRNQDNYVSWSDSEDDDEDEEIEEKEKPETNFPSPFTNILCGIIFVERRYTAVVLNRLIKEAGKQDPELAYISSNFITGHGIGKNQPRNKQMEAEFRKQEEVLRKFRAHETNLLIATSIVEEGVDIPKCNLVVRFDLPTEYRSYVQSKGRARAPISNYIMLADTDKIKSFEEDLKTYKAIEKILRNKCSKSVDTGETDIDPVMDDDDVFPPYVLRPDDGGPRVTINTAIGHINRYCARLPSDPFTHLAPKCRTRELPDGTFYSTLYLPINSPLRASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 49 - DNA, 1280 bp, Human
atggccacagcaggaaatccctggggctggttcctggggtacctcatccttggtgtcgcaggatctctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaactctggggactgggaacccatgaaattgacccccaaatacatcctgcggaaggaattcaggaatatctgttcccagcccctcctccctcaggcccaggagtccaggcccccagcccctcctccctcaaaccaagggtacagatccccagcccctcctccctcagacccaggagtccagaccccccagcccctcctccctcagacccaggagtccagcccctcctccctcagacccaggagtccagaccccccagcccctcctccctcagacccaggggtccaggcccccaacccctcctccctcagactcagaggtccaagcccccaacccctccttccccagacccagaggtccaggtcccagcccctcctccctcagacccagcggtccaatgccacctagactctccctgtacacagtgcccccttgtggcacgttgacccaaccttaccagttggtttttcattttttgtccctttcccctagatccagaaataaagtctaagagaagcgc

SEQ ID NO: 50 - protein, 254 aa, Human
MATAGNPWGWFLGYLILGVAGSLVSGSCSQIINGEDCSPHSQPWQAALVMENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQAS

SEQ ID NO: 51 - DNA, 1778 bp, Human
tagaagtttacaatgaagtttcttctaatactgctcctgcaggccactgcttctggagctcttcccctgaacagctctacaagcctggaaaaaaataatgtgctatttggtgagagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatctccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggctgtaatgttccccacctacaaatatgtcgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaaattaagtatatatattttggctcaaataaaattg

SEQ ID NO: 52 - protein, 470 aa, Human
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDLHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 53 - DNA, 2334 bp, Human
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 54 - protein, 707 aa, Human
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 55 - DNA, 803 bp, Human
gcccatagtttattataaaggtgactgcaccctgcagccaccagcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccagatcactggcaagtggttttatatcgcatcggcctttcgaaacgaggagtacaataagtcggttcaggagatccaagcaaccttcttttacttcacccccaacaagacagaggacacgatctttctcagagagtaccagacccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacgagaaggagaggaaacaggaggagggggaatcctagcaggacacagccttggatcaggacagagacttggggccatcctgcccctccaacccgacatgtgtacctcagctttttccctcacttgcatcaataaagcttctgtgtttggaacagct

SEQ ID NO: 56 - protein, 201 aa, Human
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 57 - DNA, 1630 bp, Human
aaaagcagaaagagattaccagccacagacgggtcatgagcgcggtattactgctggccctcctggggttcatcctcccactgccaggagtgcaggcgctgctctgccagtttgggacagttcagcatgtgtggaaggtgtccgacctgccccggcaatggacccctaagaacaccagctgcgacagcggcttggggtgccaggacacgttgatgctcattgagagcggaccccaagtgagcctggtgctctccaagggctgcacggaggccaaggaccaggagccccgcgtcactgagcaccggatgggccccggcctctccctgatctcctacaccttcgtgtgccgccaggaggacttctgcaacaacctcgttaactccctcccgctttgggccccacagcccccagcagacccaggatccttgaggtgcccagtctgcttgtctatggaaggctgtctggaggggacaacagaagagatctgccccaaggggaccacacactgttatgatggcctcctcaggctcaggggaggaggcatcttctccaatctgagagtccagggatgcatgccccagccaggttgcaacctgctcaatgggacacaggaaattgggcccgtgggtatgactgagaactgcaataggaaagattttctgacctgtcatcgggggaccaccattatgacacacggaaacttggctcaagaacccactgattggaccacatcgaataccgagatgtgcgaggtggggcaggtgtgtcaggagacgctgctgctcatagatgtaggactcacatcaaccctggtggggacaaaaggctgcagcactgttggggctcaaaattcccagaagaccaccatccactcagcccctcctggggtgcttgtggcctcctatacccacttctgctcctcggacctgtgcaatagtgccagcagcagcagcgttctgctgaactccctccctcctcaagctgcccctgtcccaggagaccggcagtgtcctacctgtgtgcagccccttggaacctgttcaagtggctccccccgaatgacctgccccaggggcgccactcattgttatgatgggtacattcatctctcaggaggtgggctgtccaccaaaatgagcattcagggctgcgtggcccaaccttccagcttcttgttgaaccacaccagacaaatcgggatcttctctgcgcgtgagaagcgtgatgtgcagcctcctgcctctcagcatgagggaggtggggctgagggcctggagtctctcacttggggggtggggctggcactggccccagcgctgtggtggggagtggtttgcccttcctgctaactctattacccccacgattcttcaccgctgctgaccacccacactcaacctccctctgacctcataacctaatggccttggacaccagattctttcccattctgtccatgaatcatcttccccacacacaatcattcatatctactcacctaacagcaacactggggagagcctggagcatccggacttgccctatgggagaggggacgctggaggartggctgcatgtatctgataatacagaccctgtcctttcaaaaaaaaaaaaaaaaaaaaaaaaa

SEQ ID NO: 58 - protein, 437 aa, Human
MSAVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPADPGSLRCPVCLSMEGCLEGTTEEICPKGTTHCYDGLLRLRGGGIFSNLRVQGCMPQPGCNLLNGTQEIGPVGMTENCNRKDFLTCHRGTTIMTHGNLAQEPTDWTTSNTEMCEVGQVCQETLLLIDVGLTSTLVGTKGCSTVGAQNSQKTTIHSAPPGVLVASYTHFCSSDLCNSASSSSVLLNSLPPQAAPVPGDRQCPTCVQPLGTCSSGSPRMTCPRGATHCYDGYIHLSGGGLSTKMSIQGCVAQPSSFLLNHTRQIGIFSAREKRDVQPPASQHEGGGAEGLESLTWGVGLALAPALWWGVVCPSC

SEQ ID NO: 59 - DNA, 2820 bp, Human
ggcgggttcgcgccccgaaggctgagagctggcgctgctcgtgccctgtgtgccagacggcggagctccgcggccggaccccgcggccccgctttgctgccgactggagtttgggggaagaaactctcctgcgccccagaagatttcttcctcggcgaagggacagcgaaagatgagggtggcaggaagagaaggcgctttctgtctgccggggtcgcagcgcgagagggcagtgccatgttcctctccatcctagtggcgctgtgcctgtggctgcacctggcgctgggcgtgcgcggcgcgccctgcgaggcggtgcgcatccctatgtgccggcacatgccctggaacatcacgcggatgcccaaccacctgcaccacagcacgcaggagaacgccatcctggccatcgagcagtacgaggagctggtggacgtgaactgcagcgccgtgctgcgcttcttcttctgtgccatgtacgcgcccatttgcaccctggagttcctgcacgaccctatcaagccgtgcaagtcggtgtgccaacgcgcgcgcgacgactgcgagcccctcatgaagatgtacaaccacagctggcccgaaagcctggcctgcgacgagctgcctgtctatgaccgtggcgtgtgcatttcgcctgaagccatcgtcacggacctcccggaggatgttaagtggatagacatcacaccagacatgatggtacaggaaaggcctcttgatgttgactgtaaacgcctaagccccgatcggtgcaagtgtaaaaaggtgaagccaactttggcaacgtatctcagcaaaaactacagctatgttattcatgccaaaataaaagctgtgcagaggagtggctgcaatgaggtcacaacggtggtggatgtaaaagagatcttcaagtcctcatcacccatccctcgaactcaagtcccgctcattacaaattcttcttgccagtgtccacacatcctgccccatcaagatgttctcatcatgtgttacgagtggcgttcaaggatgatgcttcttgaaaattgcttagttgaaaaatggagagatcagcttagtaaaagatccatacagtgggaagagaggctgcaggaacagcggagaacagttcaggacaagaagaaaacagccgggcgcaccagtcgtagtaatccccccaaaccaaagggaaagcctcctgctcccaaaccagccagtcccaagaagaacattaaaactaggagtgcccagaagagaacaaacccgaaaagagtgtgagctaactagtttccaaagcggagacttccgacttccttacaggatgaggctgggcattgcctgggacagcctatgtaaggccatgtgccccttgccctaacaactcactgcagtgctcttcatagacacatcttgcagcatttttcttaaggctatgcttcagtttttctttgtaagccatcacaagccatagtggtaggtttgccctttggtacagaaggtgagttaaagctggtggaaaaggcttattgcattgcattcagagtaacctgtgtgcatactctagaagagtagggaaaataatgcttgttacaattcgacctaatatgtgcattgtaaaataaatgccatatttcaaacaaaacacgtaatttttttacagtatgttttattaccttttgatatctgttgttgcaatgttagtgatgttttaaaatgtgatgaaaatataatgtttttaagaaggaacagtagtggaatgaatgttaaaagatctttatgtgtttatggtctgcagaaggatttttgtgatgaaaggggattttttgaaaaattagagaagtagcatatggaaaattataatgtgtttttttaccaatgacttcagtttctgtttttagctagaaacttaaaaacaaaaataataataaagaaaaataaataaaaaggagaggcagacaatgtctggattcctgttttttggttacctgatttccatgatcatgatgcttcttgtcaacaccctcttaagcagcaccagaaacagtgagtttgtctgtaccattaggagttaggtactaattagttggctaatgctcaagtattttatacccacaagagaggtatgtcactcatcttacttcccaggacatccaccctgagaataatttgacaagcttaaaaatggccttcatgtgagtgccaaattttgtttttcttcatttaaatattttctttgcctaaatacatgtgagaggagttaaatataaatgtacagagaggaaagttgagttccacctctgaaatgagaattacttgacagttgggatactttaatcagaaaaaaagaacttatttgcagcattttatcaacaaatttcataattgtggacaattggaggcatttattttaaaaaacaattttattggccttttgctaacacagtaagcatgtattttataaggcattcaataaatgcacaacgcccaaaggaaataaaatcctatctaatcctactctccactacacagaggtaatcactattagtattttggcatattattctccaggtgtttgcttatgcacttataaaatgatttgaacaaataaaactaggaacctgtatacatgtgtttcataacctgcctcctttgcttggccctttattgagataagttttcctgtcaagaaagcagaaaccatctcatttctaacagctgtgttatattccatagtatgcattactcaacaaactgttgtgctattggatacttaggtggtttcttcactgacaatactgaataaacatctcaccggaattc

SEQ ID NO: 60 - protein, 346 aa, Human
MFLSILVALCLWLHLALGVRGAPCEAVRIPMCRHMPWNITRMPNHLHHSTQENAILAIEQYEELVDVNCSAVLRFFFCAMYAPICTLEFLHDPIKPCKSVCQRARDDCEPLMKMYNHSWPESLACDELPVYDRGVCISPEAIVTDLPEDVKWIDITPDMMVQERPLDVDCKRLSPDRCKCKKVKPTLATYLSKNYSYVIHAKIKAVQRSGCNEVTTVVDVKEIFKSSSPIPRTQVPLITNSSCQCPHILPHQDVLIMCYEWRSRMMLLENCLVEKWRDQLSKRSIQWEERLQEQRRTVQDKKKTAGRTSRSNPPKPKGKPPAPKPASPKKNIKTRSAQKRTNPKRV

SEQ ID NO: 61 - DNA, 1723 bp, Human
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgg

SEQ ID NO: 62 - protein, 215 aa, Human
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 63 - DNA, 2020 bp, Human
gtctaaacgggaacagccctggctgagggagctgcagcgcagcagagtatctgacggcgccaggttgcgtaggtgcggcacgaggagttttcccggcagcgaggaggtcctgagcagcatggcccggaggagcgccttccctgccgccgcgctctggctctggagcatcctcctgtgcctgctggcactgcgggcggaggccgggccgccgcaggaggagagcctgtacctatggatcgatgctcaccaggcaagagtactcataggatttgaagaagatatcctgattgtttcagaggggaaaatggcaccttttacacatgatttcagaaaagcgcaacagagaatgccagctattcctgtcaatatccattccatgaattttacctggcaagctgcagggcaggcagaatacttctatgaattcctgtccttgcgctccctggataaaggcatcatggcagatccaaccgtcaatgtccctctgctgggaacagtgcctcacaaggcatcagttgttcaagttggtttcccatgtcttggaaaacaggatggggtggcagcatttgaagtggatgtgattgttatgaattctgaaggcaacaccattctccaaacacctcaaaatgctatcttctttaaaacatgtctacaagctgagtgcccaggcgggtgccgaaatggaggcttttgtaatgaaagacgcatctgcgagtgtcctgatgggttccacggacctcactgtgagaaagccctttgtaccccacgatgtatgaatggtggactttgtgtgactcctggtttctgcatctgcccacctggattctatggagtgaactgtgacaaagcaaactgctcaaccacctgctttaatggagggacctgtttctaccctggaaaatgtatttgccctccaggactagagggagagcagtgtgaaatcagcaaatgcccacaaccctgtcgaaatggaggtaaatgcattggtaaaagcaaatgtaagtgttccaaaggttaccagggagacctctgttcaaagcctgtctgcgagcctggctgtggtgcacatggaacctgccatgaacccaacaaatgccaatgtcaagaaggttggcatggaagacactgcaataaaaggtacgaagccagcctcatacatgccctgaggccagcaggcgcccagctcaggcagcacacgccttcacttaaaaaggccgaggagcggcgggatccacctgaatccaattacatctggtgaactccgacatctgaaacgttttaagttacaccaagttcatagcctttgttaacctttcatgtgttgaatgttcaaataatgttcattacacttaagaatactggcctgaattttattagcttcattataaatcactgagctgatatttactcttccttttaagttttctaagtacgtctgtagcatgatggtatagattttcttgtttcagtgctttgggacagattttatattatgtcaattgatcaggttaaaattttcagtgtgtagttggcagatattttcaaaattacaatgcatttatggtgtctgggggcaggggaacatcagaaaggttaaattgggcaaaaatgcgtaagtcacaagaatttggatggtgcagttaatgttgaagttacagcatttcagattttattgtcagatatttagatgtttgttacatttttaaaaattgctcttaatttttaaactctcaatacaatatattttgaccttaccattattccagagattcagtattaaaaaaaaaaaaaattacactgtggtagtggcatttaaacaatataatatattctaaacacaatgaaatagggaatataatgtatgaactttttgcattggcttgaagcaatataatatattgtaaacaaaacacagctcttacctaataaacattttatactgtttgtatgtataaaataaaggtgctgctttagttttcaaaaaaaaaaaaaaaaaaaaaaaaaaaaaaaaaaaaaaaaa

SEQ ID NO: 64 - protein, 379 aa, Human
MARRSAFPAAALWLWSILLCLLALRAEAGPPQEESLYLWIDAHQARVLIGFEEDILIVSEGKMAPFTHDFRKAQQRMPAIPVNIHSMNFTWQAAGQAEYFYEFLSLRSLDKGIMADPTVNVPLLGTVPHKASVVQVGFPCLGKQDGVAAFEVDVIVMNSEGNTILQTPQNAIFFKTCLQAECPGGCRNGGFCNERRICECPDGFHGPHCEKALCTPRCMNGGLCVTPGFCICPPGFYGVNCDKANCSTTCFNGGTCFYPGKCICPPGLEGEQCEISKCPQPCRNGGKCIGKSKCKCSKGYQGDLCSKPVCEPGCGAHGTCHEPNKCQCQEGWHGRHCNKRYEASLIHALRPAGAQLRQHTPSLKKAEERRDPPESNYIW

SEQ ID NO: 65 - DNA, 5494 bp, Human
gtgactcattgtgtctgtgtcgaggcgtcgggagggcctaagtccgtgtgcggtgcccttcggccggcctgagccccagagtcagctcccctttctcgcccagcgcccccaggccgctcccggggctcacggaatagtaaagaaacacatcataaaacctcccaggacataaaggtgagcacagaccctgtttggatcaagtcagttcctggagcctgaatgatgactgctgaatcacgggaagccacgggtctgtccccacaggctgcacaggagaaggatggtatcgtaatagtgaaggtggaagaggaagatgaggaagaccacatgtgggggcaggattccaccctacaggacacgcctcctccagacccagagatattccgccaacgcttcaggcgcttctgttaccagaacacttttgggccccgagaggctctcagtcggctgaaggaactttgtcatcagtggctgcggccagaaataaacaccaaggaacagatcctggagcttctggtgctagagcagtttctttccatcctgcccaaggagctccaggtctggctgcaggaataccgccccgatagtggagaggaggccgtgacccttctagaagacttggagcttgatttatcaggacaacaggtcccaggtcaagttcatggacctgagatgctcgcaagggggatggtgcctctggatccagttcaggagtcctcgagctttgaccttcatcacgaggccacccagtcccacttcaaacattcgtctcggaaaccccgcctcttacagtcacgagctcttcctgctgcccacattcctgcaccccctcatgagggtagtcccagagaccaggcgatggcatctgcactattcacagcggattcccaggcaatggtgaagatcgaggacatggctgtgtccctcattctggaggaatggggatgtcagaatctggctcggaggaatctcagtagggacaacaggcaggagaattatgggagcgcatttccccagggtggtgaaaacaggaatgagaacgaggagtcaacctcaaaggctgaaacctcggaagattcagcatcacgcggggagacaacaggaagatcccagaaagagtttggagagaaacgtgaccaggagggcaaaacaggagaaagacagcagaaaaaccctgaggagaaaaccaggaaagagaaaagagattcagggccagctataggaaaggacaaaaaaaccatcacaggagagagaggtccaagggagaaggggaaaggattgggaagaagcttcagtctgagctccaacttcaccacccctgaagaagttcccacgggaacaaagtctcacagatgtgatgaatgtggtaaatgcttcacgagaagttcaagccttatccgccataaaataatccacactggagaaaagccctatgaatgtagtgagtgtgggaaagccttcagtcttaactccaaccttgtcctgcatcagaggatccacacaggagagaaacctcatgaatgtaacgagtgtggcaaggccttcagccacagttccaatctcatcctccatcagcgcatccactctggagagaaaccttatgaatgtaatgagtgcgggaaggccttcagccagagctcggacctcaccaagcatcagagaattcacacgggggagaaaccctatgaatgtagtgaatgtggaaaagctttcaaccgaaactcatacctgattttgcatcggagaattcacactcgagaaaagccctacaagtgcactaagtgtggcaaggccttcacccgcagctccaccctcactctgcatcacagaatccatgccagagagagagcctctgagtacagcccagcctcccttgatgcatttggcgcgttcctgaaaagttgtgtgtaaaggaagaatttgccatcaagccatttcccccttttgtttctaaaattatttcagagatgtgtgctcctggagggaaaaagaaatacagcctcaacagattaaaaaacaaaagtcacacttaaggatccttctagtcacatcagcagtgttctgcctttatgtagtagttgggcatataatccttccacacagcccctgcagggaaaggctaatcttacggataatccacgtgagatttccacacaagagaaaagcacacgcatagtgaaatgtcagtcttttcagtaatgaggatacctttaaggcactcttggactctcggcaaccacaacataatagttgaaagatcaagattggctccacgaaagtgatacggaggttaggatgctacttgctgcaaacaagccctactttggccaacatcctgcttatttctcaaaaaagagggacagtgaaaacaaaaacgacattgggacatgctgctcaaggtagttatatatacgataagttgtatatatgatcactggtagcctaccaaagctgtagaaatctaggactgtgctaatcagtatcaaaccaaagatttctatctcttcccgaaagagagggtatgtgcaccagtctacagttccaaaggactgcaacaaatgtagatggttctgtcctcatccctgagatcagttctactgaaatggcaacaacaactccaaatacatctctcccttcttgaaatccctaaagcactatcgcactcctaaatgcatttctccacaagttagcacttgattgtatactgtcttttaatccttcattgtttcatgtatgaagtttttaatcaccccctaacccccaaaaaagattcggttgtcaagggatatatttctatccctgccagcacagtgctggacatagtgagtactgaacacctaagaggcactcattcagactgacttctacaggacttctacgtgtgtgataaaagcctgtgaatcgtgagtccctgaaatatggtagcctggcccagcttctaaagaggaccttcgtagccacaaggcatgctaaacctctagggtctgatgctgtttttgttccaaatataaacgaaaggcactagtcagccgcctgcatagactttctaaacagtgagtaaataccatggaatgtcagaaatgactttatcatcgtcatcttgaagaaaaatatgaacactgatgaaggtcattttttttttttgacatctttctgtggcaaagctcttagaattctttgcagttagctgctgaacagtatgatgatttggggattttctgaatcatttgtacttagatactggagttcagaagatgtgatttttgttgcttttcagaaaaaggaacgtggtagggagtgttttttcccacgcttttccagtaactttgcaaaagcattaattgttcactggcaattactaaatgtattttgttgctctgagaaactcagcagtgtactgagatgtggctggctgtgccatcgtcatagtgcacagtgacttttctgtttcttatcactaaagactgaggtgaggttatgaaactttcattggtcccatcgttgtgcgcagggtgcaactggctacctagaagctgatggcaggagactgtttcacacacaggagattgtgagctgtgtaagtagtcatcgccactcaagtaggacaagggtcctacccaaggctaggaccgccctgcggaaacagaaacatagactgagaacaaacctcaagactatcagtgtgacctccccataacaagagaaccccatatatagtttgagactttcccttgagaaacttatactaaaactattactcatcagtcattcactgatcagcagctaaagtccatgagaccaaatggttttatatggaaccaacgaagtgggagctaaaactgcacagtggtcattctttggcctctccttggctttatgacttaaaccaactacaacttccctatagcttctaagcagtttcatcagcattacttgggaaaacgtgttgcaagtcaaccagtcactaggatatttctacccatgcaacggaagaaaaacccattactccagaagagtatatctaagtaactaaaatggaggcagaaaatgttttaaggattttcttcaaagaataagccacagcaatggttttgtagaaaactcctgtgcttttgagcaaggacttttgccctctagaaagcaactgaggccaggtgcggtggctcacgcctgtaatcccagcactttgggaggccgaggtgagcggatcacctgaggtcaggagttttgagaccagcctggccaacatggtaaaatcccgtctctactaaaattataaaaattagccgggcatgatggtgggcacctgtaatcccagctacttgggaggctgaggcaggagaatcacttgaacccaggaggcagaggttgcagtgagccaagattgcacactgcactccagcctgggtgactagcaaaactccatctcaaaaaaaaaaaaaagaaagaaagaaactgaaaagtgaggtgtggaactccaggcaacgacccaagcacttattttttaagagggaaaggactttggtcatgtttacattggcctttggtttttttttttctttcagccacaactatatgctgatataactcagccatcacttttgagttttgtttttaaatgaaggttaaatgtgacctgtgccctcacatttaattccgtttcatggagacatcttcctgactttgttggatagctgccactagggttgcttcgatcttcagccacatgctgtccttgaagcagctatttgaagatgtgttttctgaggaaaacaggctacaactgtttattaaaaccacttgcagtgcattcgtttatcagatgctcagtgcaaagtgtaattgggtcatggtcaaaaacgcttgcaacatctaattggcgttcaagatagtcattcaaaagttcttggcccgctgaagtctgttaacagttgaaacattctacagttaaccattagcatgctagttgtcctaatggaaatttttgaagagtttttcaatatataccctacccttgccaggaagagaagtaaaatcctcaggttgtggggtatttgtttctactccagcctggcaggcaaggctaaaacctgagattatcgatctatgagacatcttgatatgtaaagcacttaatattaaaggcataaaagtgaaactgctaaatatgtatagagaggttgacgtgtgatacgtgggacagttcacatagacatcagagaatttattccagaaaggagcctcctgaatgtgatgaatacggcaaagcctttaatcacatctcagcccttagcatcggaaagcttatactgtaaataaacttgatgaatattatatgtgaggaaaactttcatgtatagcactcattgcttcagacagaaaatgaattccgtcggtatgttccaatcgtgatgaatttgagaaacattgcaagagggagctcaatcttggccgggcgccgtggctcacgcctgtaatcgcagcactttgggag

SEQ ID NO: 66 - protein, 563 aa, Human
MMTAESREATGLSPQAAQEKDGIVIVKVEEEDEEDHMWGQDSTLQDTPPPDPEIFRQRFRRFCYQNTFGPREALSRLKELCHQWLRPEINTKEQILELLVLEQFLSILPKELQVWLQEYRPDSGEEAVTLLEDLELDLSGQQVPGQVHGPEMLARGMVPLDPVQESSSFDLHHEATQSHFKHSSRKPRLLQSRALPAAHIPAPPHEGSPRDQAMASALFTADSQAMVKIEDMAVSLILEEWGCQNLARRNLSRDNRQENYGSAFPQGGENRNENEESTSKAETSEDSASRGETTGRSQKEFGEKRDQEGKTGERQQKNPEEKTRKEKRDSGPAIGKDKKTITGERGPREKGKGLGRSFSLSSNFTTPEEVPTGTKSHRCDECGKCFTRSSSLIRHKIIHTGEKPYECSECGKAFSLNSNLVLHQRIHTGEKPHECNECGKAFSHSSNLILHQRIHSGEKPYECNECGKAFSQSSDLTKHQRIHTGEKPYECSECGKAFNRNSYLILHRRIHTREKPYKCTKCGKAFTRSSTLTLHHRIHARERASEYSPASLDAFGAFLKSCV